(19) United States
(12) Patent Application Publication
(10) Pub. No.: US 2008/0287479 A1
(43) Pub. Date: Nov. 20, 2008

(54) INHIBITORS OF SERINE PALMITOYLTRANSFERASE

(75) Inventors: Richard Hutchings, Waltham, MA (US); William Park, Watch Hill, RI (US); Gary Bolton, Ann Arbor, MI (US); Chad Van Huis, Plymouth, MI (US); Jeff Kohrt, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/945,452

(22) Filed: Nov. 27, 2007

Related U.S. Application Data
(60) Provisional application No. 60/875,988, filed on Dec. 20, 2006.

(51) Int. Cl. A61K 31/438; C07D 401/14; A61K 31/454; A61K 31/439

(52) U.S. Cl. 514/278; 546/196; 514/323; 514/316; 514/305

(57) ABSTRACT

This invention provides compounds of the formula:

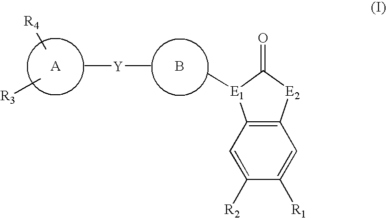

useful in the inhibition or modulation of serine palmitoyl transferase and their use in methods of treatment or amelioration of type 2 diabetes, type 1 diabetes, insulin resistance, the effects of obesity, metabolic syndrome (sometimes referred to as Syndrome X), impaired glucose tolerance, Cushing's disease, cardiovascular disease, prothrombotic conditions, myocardial infarction, hypertension, congestive heart failure, cardiomyopathy, atherosclerosis, dyslipidemia, sepsis, liver damage, retinal degenerative disorders, cachexia, emphysema, hepatitis C infections, HIV infections and inflammatory disorders and useful in methods for raising HDL plasma levels in a mammal. The compounds of this invention can also be used to prevent damage or loss of pancreatic islet beta cells (such as in the case of pancreatic beta cell apoptosis, including those related to insulin-dependent diabetes mellitus).

### FIELD OF THE INVENTION

This invention relates to indolin-2-one, benzimidazol-2-one and benzoxazol-2-one compounds useful in the inhibition or modulation of serine palmitoyl transferase, pharmaceutical compositions containing them and their use in methods of treatment or amelioration of diseases including type 2 diabetes, type 1 diabetes, insulin resistance, the effects of obesity, metabolic-syndrome, impaired glucose tolerance and cardiovascular disease or in methods of raising high density lipoprotein (HDL) plasma levels in a mammal.

### BACKGROUND OF THE INVENTION

Serine palmitoyltransferase (SPT) catalyzes the first committed step in sphingolipid synthesis. SPT condenses the palmitic acid of palmitoyl-coenzyme A with serine to produce ketosphinganine, the initial precursor to the unique aminolipid backbone that is characteristic of all sphingolipids (K. Hanada et al., J. Biol. Chem. 1997; 272(51):32108-14). SPT is composed of two different subunits, LCB1 and LCB2 (B. Weiss and W. Stoffel, Eur. J. Biochem. 1997; 249(1):239-47; see also WO 99/49021.) LCB1 and LCB2 genes are essential for cell survival and the changes in SPT activity result in a defective development of the fruit fly and filamentous fungi (J. Cheng et al., Mol. Cell. Biol. 2001; 21(18):6198-209; and T. Adachi-Yamada et al., Mol. Cell. Biol. 1999; 19(10):7276-86), and hereditary sensory neuropathy type I in humans (J. L. Dawkins et al., Nat. Genet. 2001; 27(3):309-12; and K. Bejaoui et al., Nat. Genet. 2001; 27(3):261-2).

Sphingomyelin is one of the major phospholipids in plasma lipoproteins and cell membranes. In vitro studies have demonstrated that sphingomyelin and related sphingolipids are proatherogenic in a variety of circumstances and have identified a positive correlation between plasma sphingomyelin (SM) content and the incidence of coronary artery disease (X. Jiang et al., Arterioscler. Thromb. Vasc. Biol. 2000; 20:2614-2618; and R. D. Williams, et al., J. Lipid Res. 1986. 27:763-770). SM and its derivatives are accumulated in human and experimental atherosclerotic lesions 2-(S. L. Schissel et al., J Clin Invest. 1996; 98(6):1455-64).

Although direct mechanistic links between SM and atherosclerosis have not been established, available in vitro data suggests that SM might have the following proatherogenic properties. First, increased SM content of HDL and triglyceride-rich lipoproteins, for example, is shown to obstruct reverse cholesterol transport and trigylceride-rich lipoprotein clearance by interfering with the activities of lecithin:cholesterol acyltransferase (LCAT) (D. J. Bolin and A. Jonas, J. Biol. Chem. 1996; 271(32):19152-8) and lipoprotein lipase (LPL) (I. Arimoto et al., J. Lipid Res. 1998; 39(1):143-51; I. Arimoto et al., Lipids 33:773-779 (1996); and H. Saito et al., Biochimica et Biophysica Acta 1486 (2000) 312-320), respectively. It has also been demonstrated that SM in macrophage membranes interfered with reverse cholesterol transport 19-(A. R. Leventhal et al., J. Biol. Chem. 2001; 276(48):44976-83).

Second, SM-rich lipoproteins can be converted to foam cell substrates by sphingomyelinase in the artery wall (S. L. Schissel et al., J. Biol. Chem. 1998; 273(5):2738-46), thereby promoting foam cell formation. Third, ceramide and related products of SM synthesis and breakdown are potent regulators of cell proliferation, activation and apoptosis (M. Maceyka et al., Biochim. Biophys. Acta. 2002; 1585(2-3):193-201) and hence may affect plaque growth and stability.

Other proatherogenic effects of sphingolipids include the observation that SM in LDL enhances the reactivity of LDL with sphingomyelinase, which is released by macrophages in the artery wall (Ts. Jeong et al., J. Clin. Invest. 1998; 101(4):905-912). This process results in LDL aggregation and subsequent foam cell formation (S. L. Schissel et al., J. Clin. Invest. 1996; 98(6):1455-1464). Increased sphingomyelin content in plasma membranes is also known to reduce reverse cholesterol transport by impeding the transfer of cellular cholesterol to HDL (R. Kronqvist et al., Eur. J. Biochem. 1999; 262:939-946). Furthermore, SPT activation is strongly implicated in Fas-mediated apoptosis, which could promote plaque destabilization. Fas activation causes apoptosis in macrophages (P.M. Yao and I. Tabas, J. Biol. Chem. 2000; 275:23807-23813) and smooth muscle cells (A. C. Knapp et al., Athero. 2000; 152:217-227). Fas activation depends on de novo synthesis of ceramide, a product of SPT and an SM precursor (A. Cremesti et al., J. Biol. Chem. 2001; 276:23954-23961).

Genes regulating cholesterol synthesis contain sterol regulatory elements (SREs) in their promoter regions (J. D. Horton, J. L. Goldstein and M. S. Brown, J. Clin. Invest. 2002; 109(9):1125-31). Through several intermediate steps, SREs are controlled by intra-cellular free cholesterol (M. S. Brown and J. L. Goldstein, Cell. 1997; 89(3):331-40). SM, a major plasma membrane component, has a high affinity for free cholesterol (T. S. Worgall et al., J. Biol. Chem. 200; 277(6):3878-85; and V. Puri et al., J. Biol. Chem. 2003; 278(23):20961-70). It has been reported that SM depletion by sphingomyelinase treatment causes an increased cholesterol translocation to endoplastic reticulum and suppression of SREBP cleavage (S. Sheek, M. S. Brown and J. L. Goldstein, Proc. Natl. Acad. Sci. U.S.A. 1997; 94(21):11179-83).

SPT inhibitors are known to block ceramide production and the resultant apoptosis in cardiomyocytes (D. Dyntar et al., Diabetes 2001; 50:2105-2113) and the insulin-producing pancreatic β-cells (M. Shimabukuro et al., Proc. Nat. Acad. Sci. 1998; 95(5):2498-2502). SPT inhibition prevents apoptosis of islets of prediabetic fa/fa rats (M. Shimabukuro et al., J. Biol. Chem. 1998; 273(49):32487-90). Recent findings also demonstrated that palmitate inhibits preproinsulin gene expression via ceramide biosynthesis. SPT inhibition recovered expression of preproinsulin in rat islet culture and improved the insulin production (C. L. Kelpe et al., J. Biol. Chem. 2003; 278(32):30015-21).

Myriocin is a known serine palmitoyltransferase (SPT) inhibitor (K. Hanada et al., Biochem. Pharmacol. 2000; 59:1211-1216; and J. K. Chen et al., Chemistry & Biology 1999; 6:221-235) isolated from fungi (Y. Miyake et al., Biochem. Biophys. Res. Commun. 1995; 211 (2):396-403), which is commercially available, and known to have a potent immunosuppressive activity (T. Fujita et al., J. Antibiot. (Tokyo) 1994; 47(2):208-15). It has been shown that myriocin possesses immunomodulatory properties independent of its ability to inhibit SPT and via growth inhibition in T-lymphocytes.

The article Serine palmitoyl transferase inhibitor suppresses HCV replication in a mouse model, Biochemical and Biophysical Research Communications, 2006, Jul. 21; 346(1): 67-73 describes the potential for SPT inhibition in the treatment of hepatitis C infections. Antiviral applications are also discussed in Sakamoto, H., Okamoto, K., Aoki, M., Kato, H., Katsume, A., Ohta, A., Tsukuda, T., Shimma. N., Aoki, Y., Arisawa, M., Korara, M., and Sudoh, M. Host sphingolipid biosynthesis as a target for hepatitis C virus therapy. Nature Chemical Biology 1: 333-337 (2005) and Mizrachi, Y., Harish, Z., Sundaram, S. K., and Rubinstein, A. L-Cycloserine, and inhibitor of sphingolipid biosynthesis, inhibits HIV-1 cytopathic effects, replication, and infectivity. J. Acquired Immune Deficiency Syndromes and Human Retrovology 11: 137-141 (1996).

Inhibition of ceramide activity may be useful in the treatment of emphysema, Petrache, I., Natarajan, V., Zhen, L., Medler, T. R., Richter, A. T., Cho, C., Hubbard, W. C., Berdyshev, E. V., and Tuder, R. M. Ceramide upregulation causes pulmonary cell apoptosis and emphysema-like disease in mice. Nature Medicine 11, No. 5: 491-498 (2005).

Apoptosis contributes to the multiple organ dysfunction in sepsis. Ceramide is a key meadiator of the apoptotic pathway. Thus, SPT inhibitors may be a novel therapy to decrease ceramide levels in sepsis and manage the disease, see: Power, C., Fanning, N., and Redmond, H. P. Cellular Apoptosis and organ injury in sepsis: A review. Shock 18: 197-211 (2002); Russell, J. A. Management of Sepsis. N. Engl. J. Med. 355:1699-1713 (2006); and Thevissen, K., Francois, I. E. J. A., Winderickx, J., Pannecouque, C., and Cammue, B. P. A. Ceramide involvement in apoptosis and apoptotic diseases. Mini-Rev. in Med. Chem. 6: 699-709 (2006).

Toxicities related to ionizing radiation or other cellular stresses, for example oral mucositis resulting from ionizing radiation used to treat head and neck cancers, may be treated with SPT inhibitors, see Hwang, D., Popat, R., Bragdon, C., O'Donnell, K. E., Sonis, S. T. Effects of ceramide inhibition on experimental radiation-induced oral mucositis. Oral Surg. Med. Oral Pathol. Radiol. Endod. 100: 321-329 (2005).

The use of enzymes of sphingolipid metabolism in the therapeutic management of retinal degeneration is discussed in Acharya, U., Patel, S., Koundakjian, E., Nagashima, K., Han, X., and Acharya, J. K. Modulating sphingolipid biosynthetic pathway rescues photoreceptor degeneration. Science 299:1740-1743 (2003).

A review of the contribution aberrant ceramide accumulation to the pathologies of metabolic Syndrome X, type 2 diabetes and Cushing's disease, including insulin resistance, atherosclerosis, susceptibility to infection, poor wound healing and hypertension are discussed in Summers, S. A. and Nelson, D. H. A role for sphingolipids in producing the common features of type 2 diabetes, metabolic syndrome X and Cushing's syndrome. Diabetes 54: 591-602 (2005).

The proatherogenic role for sphingolipids, especially in the context of metabolic syndrome, is discussed in Rekhter, M. and Karathanasis, S. Sphingolipids in atherosclerosis: a metabolic underpinning of vascular disease. Future Lipidol. 1 (5):605-614 (2006).

WO 01/80903 discloses detection and treatment of atherosclerosis based on plasma sphingomyelin concentration. WO 02/074924 and U.S. 2002/0197654, Thromb. Haemost., 2001; 86:1320-1326; disclose a method for comparatively measuring the level of normal and hyperproliferative serine palmitoyltransferase expression in a mammalian cell and uses thereof, such as detecting cancer or treating restenosis. U.S. 2003/9996022 discloses methods and compositions useful for treating or preventing cardiovascular or cerebrovascular disease through the use of agents that interfere with the production and/or biological activities of sphingolipids and their metabolites, particularly sphingosine (SPH) and sphingosine1-phosphate (S-1-P). WO 01/80715 discloses methods for identifying compounds useful for preventing acute clinical vascular events in a subject. U.S. Pat. No. 6,613,322; US2003/0026796 and WO 99/11283 discloses methods for treating a subject suffering from an atherosclerotic vascular disease comprising administering to the subject an amount of a zinc sphingomyelinase inhibitor effective to decrease extracellular zinc sphingomyelinase activity in the subject.

In view of these physiological activities of SPT, there remains a need to identify SPT inhibitors useful as pharmaceutical agents.

### DESCRIPTION OF THE INVENTION

This invention comprises compounds of the formula:

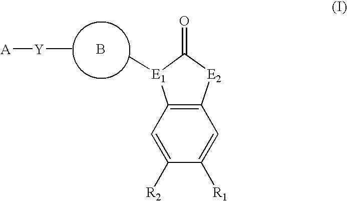

wherein:
E₁ is selected from N or CH;
E₂ is selected from NR, O or CRₐR_{b};
R is H, C₁-C₃ alkyl, —CH₂—COOH, —CH₂—COO—C₁-C₆ alkyl;

Rₐ and R_{b} are independently selected from H or C₁-C₃ alkyl;

Y is selected from the linking groups:

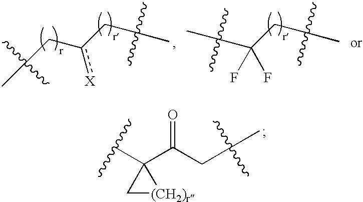

the dashed line [symbol] connected to X indicates an optional double bond;

r is an integer of from 0 to 2;

r′ is an integer from 1 to 3;

r″ is an integer from 1 to 3;

X is H, halogen, OH, oxo or ═NOR′; wherein R′ is hydrogen, C₁-C₆ straight or branched chain alkyl, C₃-C₆ cycloalkyl or —C₁-C₃ alkyl)-C₃-C₆ cycloalkyl;

The B ring represents a moiety selected from the group of:

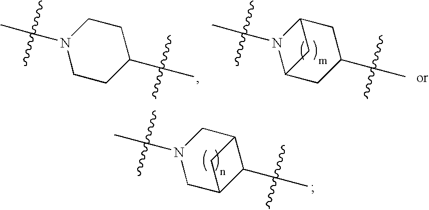

m and n are each independently 0 to 2;

A is a C₁-C₆ alkyl, C₂-C₆ alkenyl, a carbocycle, or a heterocycle; each of the alkyl, alkenyl, carbocycle and heterocycle groups being optionally substituted by R₃ and R₄;

R₁ is selected from:
a) H, halogen, CN, —C(O)R⁵, —C(O)OR⁵, —C(O)NR⁵R⁶, —S(O)ₚR⁵, S(O)₂N R⁵R⁶ and C₁-C₃ alkyl optionally substituted by OH; or
b) a moiety of the formula:

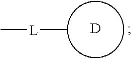

L is a linking group selected from a bond, a C₁-C₃ alkylene group, —C(O)—, —C(O)NR⁵—, —C(O)O—, —S(O)ₚ— and —S(O)₂NR⁵—;

p is 0 to 2;

D represents a moiety selected from —(CH₂)₀₋₃-carbocycle and —(CH₂)₀₋₃-heterocycle; each optionally substituted by from 1 to 3 groups independently selected from OH, oxo, CN, NH₂, NO₂, CF₃, halogen, C₁-C₃ alkyl, —O—C₁-C₃ alkyl, —S—C₁-C₃ alkyl, —NHC(O)R⁵, —O—C₁-C₃ alkyl, —S—C₁-C₃ alkyl, —(CH₂)₀₋₃—C(O)R⁵, —(CH₂)₀₋₃—C(O)OR⁴, —(CH₂)₀₋₃—C(O)NR⁵R⁶—S(O)ₚR⁵, and S(O)₂N R⁵R⁶; wherein the C₁-C₃ alkyl, —O—C₁-C₃ alkyl, and —S—C₁-C₃ alkyl groups may be substituted by OH;

R⁵ is selected from H, C₁-C₆ alkyl, and —(CH₂)₀₋₃—(C₃-C₇ cycloalkyl); wherein the C₁-C₆ alkyl, and —(CH₂)₀₋₃—(C₃-C₇ cycloalkyl) groups may be optionally substituted by from 1 or 3 groups selected from OH, —O—C₁-C₃ alkyl, —S—C₁-C₃ alkyl, —COOR⁶, —NH₂, —NH(C₁-C₆ alkyl), —N(C₁-C₆ alkyl)₂, halogen, CF₃, CN, —NC(O)R⁶, and —OC(O)R⁶;

R⁶ is selected from H, C₁-C₆ alkyl, C₁-C₆ alkenyl, —(CH₂)₀₋₃-carbocycle and —(CH₂)₀₋₃-heterocycle;

R₂ is selected from the group of H, halogen, CF₃, C₁-C₃ alkoxy, C₁-C₃ alkyl and C₂-C₃ alkenyl; the C₁-C₃ alkoxy, C₁-C₃ alkyl and C₂-C₃ alkenyl groups being optionally substituted by from 1 to 3 halogen, OH, C₁-C₃ alkoxy or CN groups;

R₃ and R₄ are independently selected from H, halogen, CN, OH, oxo, C₁-C₆ alkyl, C₃-C₆ cycloalkyl, —CH₂—C₃-C₆ cycloalkyl, C₁-C₃ haloalkyl, —O—C₁-C₆ alkyl, —S—C₁-C₆ alkyl, phenyl, benzyl, NR_{y}R_{z}, —C(O)NR_{y}R_{z}, COOR_{y}S, —C₁-C₃ alkyl-OH, —S(O)_{r′″}—R_{y};

R_{y} and R_{z} are independently selected from H, C₁-C₆ alkyl, C₃-C₆ cycloalkyl, —CH₂—C₃-C₆ cycloalkyl, phenyl, benzyl,

r′″ is an integer selected from 1 or 2;

or R₃ and R₄ together form a fused or spiro 3-, 4-, 5- or 6-membered carbocyclic or heterocyclic ring;

or a pharmaceutically acceptable salt form thereof.

The terms spiro, spirocycle or spirocyclic used herein refer to two rings joined by a carbon atom common to both.

The terms carbocycle or carbocyclic used herein indicates a fully saturated, partially saturated or unsaturated C₃-C₁₀ monocyclic or bicyclic ring having only carbon ring atoms. The bicyclic carbocycles may be fused or spirocyclic ring systems. The C₃-C₁₀ carbocyclic groups include fully saturated cycloalkyl rings such as cyclopropyl, cyclobutyl, cyclopentyl, cyclohexyl, cycloheptyl and cyclooctyl rings; partially saturated carbocyclic groups such as cyclopropene, cyclobutene, cyclopentene and cyclohexene rings; bicyclic moieties such as indene, 2,3-dihydro-indene and 1,2,3,4-tetrahydronaphthalene groups; bridged moieties such as bicyclo[3.1.0]hexane, bicyclo[3.2.1]octane and bicyclo[3.1.1]heptane groups; and spirocyclic carbocycles, such as spiro[2.3]hexane, spiro[2.4]hexane, spiro[3.3]heptane, spiro[3.4]octane, spiro[2.5]octane, spiro[4.5]decane and spiro[5.5]hendecane. Unsaturated carbocyclic moieties include phenyl and naphthyl groups.

The terms heterocycle or heterocyclic used herein indicates a fully saturated, partially saturated or unsaturated 3- to 12-membered monocyclic or bicyclic ring having from 1 to 5 ring heteroatoms selected from O, S or N. The bicyclic heterocycles may be fused or spirocyclic ring systems. Monocyclic or bicyclic heterocycles, alone and together with fused or spirocyclic groups formed from R₃ and R₄, described above include aziridines, oxirane, azetidine, azirine, thirene, oxetane, oxazetidine, tetrazole, oxadiazole, thiadiazole, triazole, isoxazole oxazole, oxathiazole, oxadiazolone, isothiazole, thiazole, imidazole, pyrazole, isopyrazole, diazine, oxazine, dioxazine, oxadiazine, thiadiazine, oxathiazole, triazine, thiazine, dithiazine, tetrazine, pentazine, pyrazolidine, pyrrole, pyrrolidine, furan, thiophene, isothiophene, tetrazine, triazine, morpholine, thiazine, piperazine, pyrazine, pyridazine, pyrimidine, piperidine, pyridine, pyran, thiopyran, azepine, diazepine, triazepine, azepane, 3-aza-bicylco[3.2.1]octane, 2-aza-bicylco[2.2.1]heptane, octahydrocyclopentapyrrole, aza-bicyclo-nonane, indole, indoline, isoindoline, indolizine, octahydro-isoindole, 2-azaspiro[4.5]decane, 6-azaspiro[2.5]octane, 7-azaspiro[3.5]nonane, 8-azaspiro[4.5]decane, 3-asaspiro[5.5]undecane, 1-oxa-7-azaspiro[4.4]nonane, 1-oxa-8-azaspiro[4.5]decane, purine, benzothiazole, benzoxazole, indazole, benzofuran, and isobenzofuran. Examples of spirocyclic heterocycles include oxaspiro[2.3]hexane, 1-oxaspiro[3.4]octane, 1-oxaspiro[2.5]octane, 2-oxaspiro[4.5]decane, 2,6-diazaspiro[3.2]heptane, azaspiro[2.5]octane, 6-aza-spiro[2.5]octane, 1,6-diaza-spiro[2.5]octane, 7-aza-spiro[3.5]nonane, 3-aza-spiro[5.5]undecane, 8-azaspiro[4.5]decane, 1,3-diazaspiro[4.5]decane, 2,8-diazaspiro[5.5]hendecane, 3,9-diazaspiro[5.5]hendecane and 1-ox-6-azaspiro[2.5]octane.

It will be understood that the terms listed above for heterocycles includes each possible atomic orientation for the groups listed. For instance, the term oxadiazole includes 1,2,3-oxadiazole, 1,3,4-oxadiazole and 1,2,4-oxadiazole; the term thiadiazole includes 1,2,3-thiadiazole, 1,2,4-thiadiazole, 1,2,5-thiadiazole and 1,3,4-thiadiazole; and the term diazepine includes 1,3-diazepine and 1,4-diazepine groups.

The terms halo or halogen refer to F, Cl, Br or I. The term haloalkyl refers to an alkyl group having from 1 halogen substituent to the maximum number of halogen substituents allowed by valence requirements. Examples include —CH₂F, —CF₃, —CH₂—CF₃, —CF₂—CF₃, etc.

Alkyl groups, alkenyl groups and the alkyl portions of alkoxy and thioalkoxy groups discussed herein include straight or branched groups having the number of carbon atoms indicated including, for example, methyl, methoxy, thiomethyl, ethyl, styrene, propyl, isopropyl, isopropyloxy, allyl, n-butyl, t-butyl, isobutyl, penyl, isopentyl, and 2-methylbutyl groups.

R₃ and R₄ can together with a carbon atom to which they are bound form 3-6 membered carbocyclic or heterocyclic rings. The heterocyclic spirocycle rings may have from 1 to 3 ring heteroatoms selected from O, S or N, examples of which include fused morpholine, pyran, tetrahydropyran, tetrahydrofuran and dihydrofuran groups.

It will be understood that instances wherein m or n is 0 indicate a single bond between the 3- and 5-position carbon atoms or the 2- and 6-position carbon atoms of the ring representing B in the description, i.e. a 3-aza-bicyclo[3.1.0]hexane ring or 6-aza-bicyclo[3.1.0]hexane, respectively.

Another group of compounds are those of Formula (II):

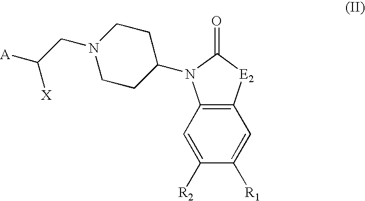

wherein A, X, R₁, R₂ and E₂ are as defined above for Formula (I); or a pharmaceutically acceptable salt, hydrate or solvate thereof.

A further group of compounds are those of Formula (III):

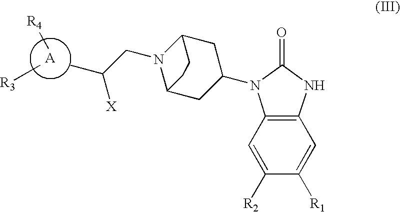

wherein X, R₁, R₂, R₃, R₄ and A are as defined above for Formula (I), above; or a pharmaceutically acceptable salt, hydrate or solvate form thereof.

A group of compounds are those of the formula IV:

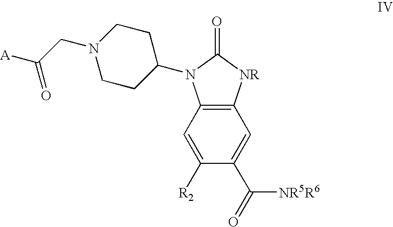

wherein:

R is H or C₁-C₃ alkyl;

R₂ is selected from the group of H, halogen, CF₃, C₁-C₃ alkoxy, C₁-C₃ alkyl and C₂-C₃ alkenyl; the C₁-C₃ alkoxy, C₁-C₃ alkyl and C₂-C₃ alkenyl groups being optionally substituted by from 1 to 3 halogen, OH, C₁-C₃ alkoxy or CN groups;

R⁵ is selected from H, C₁-C₆ alkyl, and —(CH₂)₀₋₃—(C₃-C₇ cycloalkyl); wherein the C₁-C₆ alkyl, and —(CH₂)₀₋₃—(C₃-C₇ cycloalkyl) groups may be optionally substituted by from 1 or 3 groups selected from OH, —O—C₁-C₃ alkyl, —S—C₁-C₃ alkyl, —COOR⁶, —NH₂, —NH(C₁-C₆ alkyl), —N(C₆-C₆ alkyl)₂, halogen, CF₃, CN, —NC(O)R⁶, and —OC(O)R⁶;

R⁶ is selected from H and C₁-C₆ alkyl;

A is a 5- or 6-membered carbocycle or heterocycle; each being optionally substituted by R₃ and R₄;

R₃ and R₄ are independently selected from H, halogen, CN, OH, oxo, C₁-C₆ alkyl, C₃-C₆ cycloalkyl, —CH₂—C₃-C₆ cycloalkyl, C₁-C₃ haloalkyl, —O—C₁-C₆ alkyl, —S—C₁-C₆ alkyl, NR_{y}R_{z}, —C(O)NR_{y}R_{z}, COOR_{y}, and —C₁-C₃ alkyl-OH;

R_{y} is selected from H, C₁-C₆ alkyl, C₃-C₆ cycloalkyl and —CH₂—C₃-C₆ cycloalkyl,

R₁ is H or C₁-C₆ alkyl;

or R₃ and R₄ together form a fused or spiro 3-, 4-, 5- or 6-membered carbocyclic or heterocyclic ring;

or a pharmaceutically acceptable salt thereof.

Within the group of compounds of Formula IV is another group wherein:

R is H or C₁-C₃ alkyl;

R₂ is selected from the group of halogen, CF₃, C₁-C₃ alkoxy, C₁-C₃ alkyl; with the C₁-C₃ alkoxy and C₁-C₃ alkyl groups being optionally substituted by from 1 to 3 halogen, OH, C₁-C₃ alkoxy or CN groups;

R⁵ is selected from H, C₁-C₆ alkyl, and —(CH₂)₀₋₃—(C₃-C₇ cycloalkyl); wherein the C₁-C₆ alkyl, and —(CH₂)₀₋₃—(C₃-C₇ cycloalkyl) groups may be optionally substituted by from 1 or 3 groups selected from OH, —O—C₁-C₃ alkyl, —S—C₁-C₃ alkyl, —COOR⁶, —NH₂, —NH(C₁-C₆ alkyl), —N(C₁-C₆ alkyl)₂, halogen, CF₃, CN, —NC(O)R⁶, and —OC(O)R⁶;

R₆ is selected from H and C₁-C₆ alkyl;

A is a 5- or 6-membered cycloalkyl group optionally substituted by R₃ and R₄;

R₃ and R₄ are independently selected from H, halogen, C₁-C₆ alkyl, C₁-C₃ haloalkyl, —O—C₁-C₆ alkyl, and —C₁-C₃ alkyl-OH;

or R₃ and R₄ together form a fused or spiro 3-, 4-, 5- or 6-membered carbocyclic or heterocyclic ring;

or a pharmaceutically acceptable salt thereof.

Within each of the groups above represented by Formulas IV is a further group of compounds wherein R is hydrogen; R₂ is halogen or CF₃; R⁵ and R⁶ are independently selected from H and C₁-C₆ alkyl; A is a cyclohexyl group substituted by R₃ and R⁴; R₃ and R⁴ are independently selected from H, halogen, C₁-C₆ alkyl, C₁-C₃ haloalkyl, —O—C₁-C₆ alkyl, and —C₁-C₃ alkyl-OH; or R₃ and R₄ together form a spiro 3-, 4-, 5- or 6-membered carbocyclic; or a pharmaceutically acceptable salt thereof.

Another group of compounds are those of Formula V:

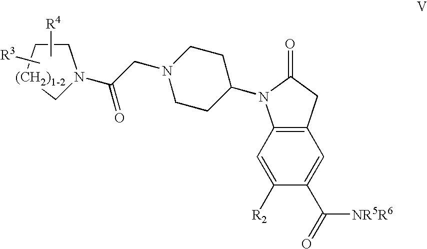

wherein R₂, R₃, R₄, R⁵ and R⁶ are as defined for Formula IV, above; or a pharmaceutically acceptable salt thereof.

Within the group of compounds represented by Formula V are compounds wherein:

R₂ is selected from the group of halogen, CF₃, C₁-C₃ alkoxy, C₁-C₃ alkyl; with the C₁-C₃ alkoxy and C₁-C₃ alkyl groups being optionally substituted by from 1 to 3 halogen, OH, C₁-C₃ alkoxy or CN groups;

R⁵ is selected from H, C₁-C₆ alkyl, and —(CH₂)₀₋₃—(C₃-C₇ cycloalkyl); wherein the C₁-C₆ alkyl, and —(CH₂)₀₋₃—(C₃-C₇ cycloalkyl) groups may be optionally substituted by from 1 or 3 groups selected from OH, —O—C₁-C₃ alkyl, —S—C₁-C₃ alkyl, —COOR⁶, —NH₂, —NH(C₁-C₆ alkyl), —N(C₁-C₆ alkyl)₂, halogen, CF₃, CN, —NC(O)R⁶, and —OC(O)R⁶;

R⁶ is selected from H and C₁-C₆ alkyl;

the moiety:

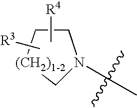

represents a (3aS,6aR)-octahydrocyclopenta[c]pyrrole group or a piperidine or pyrrolidine group substituted by R₃ and R₄;

R₃ and R₄ are independently selected from H, halogen, C₁-C₆ alkyl, C₁-C₃ haloalkyl, —O—C₁-C₆ alkyl, and —C₁-C₃ alkyl-OH;

or R₃ and R₄ together form a fused or spiro 3-, 4-, 5- or 6-membered carbocyclic or heterocyclic ring;

or a pharmaceutically acceptable salt thereof.

Within each of the groups above represented by Formula V is a further group of compounds wherein R₂ is H, halogen or CF₃; R⁵ and R⁶ are independently selected from H and C₁-C₆ alkyl; A is a (3aS,6aR)-octahydrocyclopenta[c]pyrrole group or a piperidine or pyrrolidine group substituted by R₃ and R₄; R₃ and R₄ are independently selected from H, halogen, C₁-C₆ alkyl, C₁-C₃ haloalkyl, —O—C₁-C₆ alkyl, and —C₁-C₃ alkyl-OH; or R₃ and R₄ together form a spiro 3-, 4-, 5- or 6-membered carbocyclic; or a pharmaceutically acceptable salt thereof. Further groups within those described above represented by Formula V are those in which R₂ and R₅ are hydrogen; and R₆ is C₁-C₆ alkyl; or a pharmaceutically acceptable salt thereof.

Within each of the groups described herein is another group wherein R₁ is —C(O)NH₂, —C(O)NH(C₁-C₆ alkyl), —C(O)N(C₁-C₆ alkyl)₂, —C(O)NH—(CH₂)₀₋₃—(C₃-C₇ cycloalkyl), or a heterocycle, as defined above, having at least one ring nitrogen atom. Non-limiting examples of nitrogen-containing heterocycles include aziridine, azetidine, azirine, oxazetidine, tetrazole, oxadiazole, thiadiazole, triazole, isoxazole oxazole, oxathiazole, oxadiazolone, isothiazole, thiazole, imidazole, pyrazole, isopyrazole, diazine, oxazine, dioxazine, oxadiazine, thiadiazine, oxathiazole, triazine, thiazine, dithiazine, tetrazine, pentazine, pyrazolidine, tetrazine, triazine, morpholine, thiazine, piperazine, pyrazine, pyridazine, pyrimidine, piperidine, pyridine, azepine, diazepine, triazepine, azepane, 3-aza-bicylco[3.2.1]octane, 2-aza-bicylco[2.2.1]heptane, aza-bicyclo-nonane, indole, indoline, isoindoline, indolizine, octahydro-isoindole, 2-azaspiro[4.5]decane, 6-azaspiro[2.5]octane, 7-azaspiro[3.5]nonane, 8-azaspiro[4.5]decane, 3-asaspiro[5.5]undecane, 1-oxa-7-azaspiro[4.4]nonane, 1-oxa-8-azaspiro[4.5]decane, purine, indazole, 2,6-diazaspiro[3.2]heptane, azaspiro[2.5]octane, 6-aza-spiro[2.5]octane, 1,6-diaza-spiro[2.5]octane, 7-aza-spiro[3.5]nonane, 3-aza-spiro[5.5]undecane, 8-azaspiro[4.5]decane, 1,3-diazaspiro[4.5]decane, 2,8-diazaspiro[5.5]hendecane, 3,9-diazaspiro[5.5]hendecane and 1-ox-6-azaspiro[2.5]octane.

Another aspect of this invention is directed to compounds of formula II as described above wherein

E₂ is CRₐR_{b};

Rₐ, and R_{b} are H;

R₂ is H or fluoro;

R₁ is —C(O)NR⁵R′;

R⁵ and R^{r} are H;

X is oxo;

R₃ and R₄ are H; and

A is octahydro-cyclopentapyrrole;

or a pharmaceutically acceptable salt thereof.

A preferred compound is 1-(1-{2-[hexahydrocyclopenta[c]pyrrol-2(1H)-yl]-2-oxoethyl}piperidin-4-yl)-N-methyl-2-oxoindoline-5-carboxamide or a pharmaceutically acceptable salt thereof.

A preferred compound is 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide or a pharmaceutically acceptable salt thereof.

A preferred salt has the structure

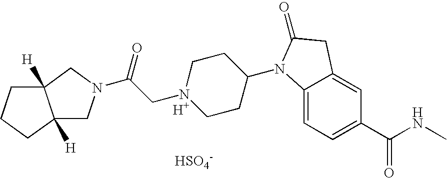

### BRIEF DESCRIPTION OF THE DRAWINGS

FIG. 1 is a characteristic x-ray powder diffraction pattern showing that the compound of Example 20 prepared by the first process, (1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium hydrogen sulfate), is crystalline. (Vertical Axis: Intensity (Counts); Horizontal Axis: Two theta (degrees))

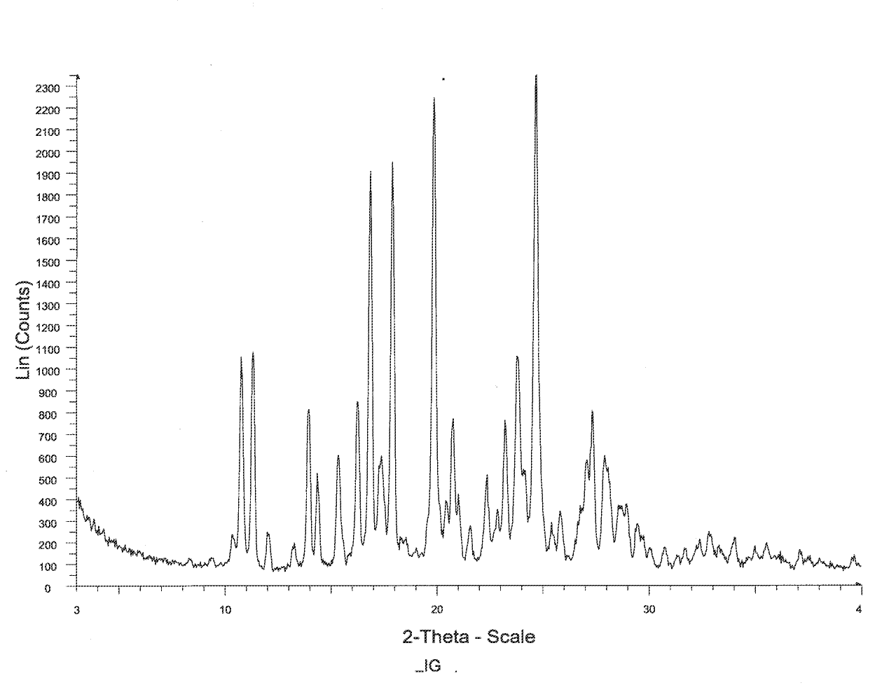

The compounds herein are useful in the inhibition or modulation of serine palmitoyl transferase in a mammal. They may be used in methods of treatment or amelioration of type 2 diabetes, type 1 diabetes, insulin resistance, the effects of obesity, metabolic syndrome (sometimes referred to as Syndrome X), Cushing's disease, impaired glucose tolerance, cardiovascular disease, atherosclerosis, prothrombotic conditions, myocardial infarction, hypertension, congestive heart failure, cardiomyopathy, atherosclerosis, dyslipidemia, retinal degenerative disorders, emphysema, sepsis, liver damage, cachexia, viral infections including hepatitis C and human immunodeficiency virus (HIV) and inflammatory disorders. The compounds of this invention can also be used to prevent damage or loss of pancreatic islet beta cells (such as in the case of pancreatic beta cell apoptosis, including those related to insulin-dependent diabetes mellitus). The use of the present compound in each of these methods comprises administering to a mammal in need of such treatment a pharmaceutically effective amount of a compound described herein, or a pharmaceutically acceptable salt, hydrate or solvate form thereof.

The compounds herein may also be used to treat, minimize or prevent liver damage from viral, alcohol related or reperfusion injuries. They may also be beneficial for protection of tissues in preparation of, during and following transplantation, such as in islet transplantation or liver transplantation. The compounds herein may be used to preserve liver cells and tissues in culture.

This invention also comprises a method of raising high density lipoprotein (HDL) plasma levels in a mammal, the method comprising administering to a mammal in need of such treatment a pharmaceutically effective amount of a compound described herein, or a pharmaceutically acceptable salt, hydrate or solvate form thereof.

Pharmaceutically acceptable salts of the compounds of the compounds herein include the acid addition and base salts (including disalts) thereof and can be made my methods well known in the art. Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. For a review on suitable salts, see “Handbook of Pharmaceutical Salts: Properties, Selection, and Use” by Stahl and Wermuth (Wiley-VCH, Weinheim, Germany, 2002). A pharmaceutically acceptable salt of these compounds may be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized. The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

The compounds herein include polymorphic and isomeric forms thereof (including optical, geometric and tautomeric isomers) and their isotopically-labelled forms.

The compounds of the present invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. [Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).]

Prodrugs can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985). Some examples of such prodrugs include: (i) where the compounds herein contain a carboxylic acid functionality (—COOH), an ester thereof, for example, replacement of the hydrogen with (C₁-C₈)alkyl or benzyl group; (ii) where the compound contains an alcohol functionality (—OH), an ether thereof, for example, replacement of the hydrogen with (C₁-C₆)alkanoyloxymethyl; and (iii) where the compound contains a primary or secondary amino functionality (—NH₂ or —NHR where R≠H), an amide thereof, for example, replacement of one or both hydrogens with (C₁-C₁₀)alkanoyl.

Compounds herein containing one or more asymmetric carbon atoms can exist as two or more stereoisomers. Where a compound contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism. Included within the scope of the claimed compounds present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine. Cis/trans isomers may be separated by conventional techniques well known to those skilled in the art, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Mixtures of stereoisomers may be separated by conventional techniques known to those skilled in the art. [see, for example, “Stereochemistry of Organic Compounds” by E L Eliel (Wiley, New York, 1994).]

As used herein, the terms “treat” or “treatment” are used interchangeably and are meant to indicate a postponement of development of diseases and/or a reduction in the severity of such symptoms that will or are expected to develop. The terms further include ameliorating existing disease symptoms, preventing additional symptoms, and ameliorating or preventing the underlying metabolic causes of symptoms.

Depending on the disease and condition of the patient, the term “treatment” as used herein may include one or more of curative, palliative and prophylactic treatment. Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

Pharmaceutically useful formulations comprising a pharmaceutically effective amount of one or more compounds herein, or a pharmaceutically acceptable salt thereof, and a pharmaceutically acceptable carrier or excipient may also be prepared by well known methods. Examples of such carriers, excipients and methods may be seen in Remington's Pharmaceutical Sciences. Suitable formulations include oral, topical, transdermal, systemic and parenteral formulations. For example, useful dosage forms include pills, tablets, capsules, powders, granules, elixirs, tinctures, syrups, emulsions, solutions, suspensions, edible films, transdermal forms, injectables, intravenous and other conventional dosage forms all using methods known in the pharmaceutical arts. The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavoring agents, preservatives and taste-masking agents. Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may also be encapsulated. The formulation of tablets is well known in the art. A discussion is found in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release.

Suitable modified release formulations for the purposes herein are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds herein may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

The compounds herein may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, J Pharm Sci, 88 (10), 955-958 by Finnin and Morgan (October 1999).]

For administration to human patients, the total daily dose of the compounds herein is typically in the range of from about 10 mg to about 750 mg depending, of course, on the mode of administration, in the range of from about 50 mg to about 600 mg or in the range of from about 100 mg to about 500 mg. The total daily dose may be administered in single or divided doses. These dosages are based on an average human subject having a weight of about 65 kg to 70 kg. The physician will readily be able to determine doses for subjects whose weight falls outside this range, such as infants and the elderly.

The compounds herein may be used in the methods described above in combination with pharmaceutically useful dosages of agents known in the art for treating such conditions, including insulin, insulin analogs, incretin, incretin analogs and mimetics (such as exenetide, liraglutide and CJC-1131), glucagon-like peptide, glucagon-like peptide analogs, exendin, exendin analogs, PACAP and VIP analogs, sulfonylureas (such as tolbutamide, tolazamide, acetohexamide, gliclazide, chlorpropamide, glipizide, glyburide, gliquidone and glimepiride), biguanides (such as metformin), cr-glucosidase inhibitors, Acetyl-CoA Carboxylase inhibitors, caspase inhibitors, and PPAR ligands (such as the thiazolidinediones pioglitazone, rosiglitazone, troglitazone).

These known agents may be administered in conjunction with the compounds described herein at the dosages and in the regimens for which they are presently used. Descriptions of the dosages and regimens are readily available in the art, such as in the 2006 Physicians' Desk Reference (60 Edition, ISBN 1563635267), published by Thompson PDR, Montvale, N.J. 07645-1742.

### Preparation of the Compounds of the Invention

The present invention contains compounds that can be prepared in a variety of ways to one skilled in organic synthesis. The compounds outlined herein can be synthesized according to the methods described below, along with methods typically used by a synthetic organic chemist, and combinations or variations of those methods, which are generally known to one skilled in the art of synthetic chemistry. The synthetic route of compounds in the present invention is not limited to the methods outlined in the schemes shown below. One skilled in the art will be able to use the synthetic schemes presented below to synthesize compounds claimed in this invention. Individual compounds may require manipulation of the reaction conditions in order to facilitate various functional groups. Various protecting groups known to one skilled in the art may be required. Purification, if necessary, may be accomplished on a silica gel column eluted with the appropriate organic solvent system. Also, reverse phase HPLC or recrystallization may be employed. The following non-limiting descriptions also demonstrate methods for the synthesis of compounds of the invention. General detection methods used in the descriptions herein: ¹H NMR spectral data herein were obtained by using a Varian 400U (400 MHz nuclear magnetic resonance) instrument. Liquid chromatography mass spectroscopy (LCMS) data were obtained using Agilent 1100 Series LC/MSD system, Retention time (in min) for compounds with chromophore was determined by employing a standard Analytical LC Method: Gradient=0-100% solvent B over 4 minutes. Flow rate=1.5 mL/minute. Solvent A=water with 0.1% trifluoroacetic acid, Solvent B=acetonitrile with 0.1% trifluoroacetic acid. Column=Phenomenex Develosil Combi RP3 50×4.6 mm, 45° C. Positive and negative ion atmospheric pressure chemical ionization (APCI) mass spectra were obtained on a Micromass Platform LC mass spectrometer operating in Open Access mode. Samples were introduced by loop injection using a Gilson 215 autosampler into a mobile phase of 80:20 acetonitrile:water flowing at 200 μL/min delivered by a Hewlett-Packard HP1100 HPLC. The mass spectrometer source and probe temperatures were 150° C. and 450° C., respectively. The cone voltage was 10-30 V depending on the desired fragmentation patterns while the corona pin was held at 3.5 kV in positive ion and 3.0 kV in negative ion mode.

Scheme 1 describes a general method for the preparation of benzimidazole compounds herein.

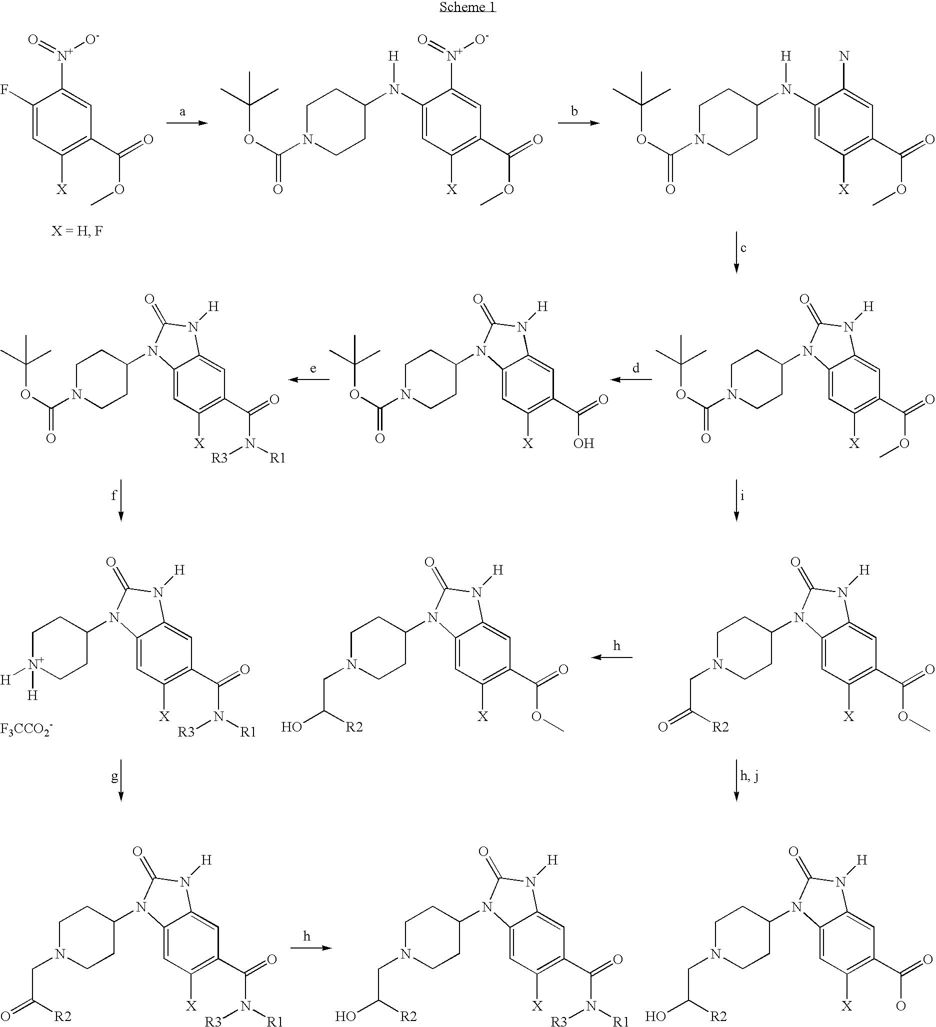

Reaction conditions: a) 4-Amino-1-BocN-piperidine, DIEA, DMF b) Raney Ni/CH₂Cl₂ c) CDl/CH₂Cl₂ rt, d) NaOH/MeOH e) HNR₁R₃, EDC, HOBt DMF, f) TFA/CH₂Cl₂, rt, g) R₂C(O)CH2-Cl or Br, DIEA, DMF rt, h) NaBH₄, EtOH, i) TFA/DCM, then R₂C(O)CH₂Cl, DIEA, DMF rt, j) NaOH/MeOH

### Preparation of 4-Fluoro-3-nitro-benzoic acid methyl ester

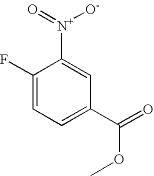

An ice-cold solution of acid (4-Fluoro-3-nitro-benzoic acid, 25.0 g, 135.2 mmole) (Chem. Abstr. Reg. No. 329-59-9) in a mixture of dichloromethane (DCM, 250 ml) and methanol (MeOH, 70 ml) was treated dropwise with a trimethylsilyidiazomethane solution (2.0M in hexanes) until bubbling stopped (˜95 ml). The reaction mixture was stirred for 1 hour and concentrated to yield a slightly yellow solid which was dried on a vacuum pump at room temperature to yield solid. (26.7 g, 99%) Experimental Data; LCMS 50% H₂O, Retention time 1.75 min., C₈H₆FNO₄, MW 199.1; Found APCl⁻ 199.3 (M+); ¹H NMR (CDCl₃) δ 8.60-8.57 (m, 1H), 8.18-8.14 (m, 1H), 7.25-7.20 (m, 1H), 3.82 (s, 3H).

### Preparation of tert-butyl 4-{[4-(methoxycarbonyl)-2-nitrophenyl]amino}piperidine-1-carboxylate

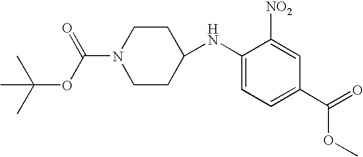

A solution of 4-fluoro-3-nitro-benzoic acid methyl ester (10.0 g (50.22 mmole) in dimethylformamide (DMF) (100 ml) was treated with 4-Amino-piperidine-1-carboxylic acid tert-butyl ester (10.69 g, 51.22 mmole) followed by diisopropylethylamine (DIEA, 17.49 ml, 100.4 mmole). The mixture was stirred overnight at room temperature. The reaction was stirred at −70° C. for 1 hour and concentrated to form a residue. The residue was taken up in Dichloromethane (DCM) and washed with water (4×100 ml). The organics were dried over Na₂SO₄, filtered, and concentrated to yield a yellow solid. The yellow solid was chromatographed on silica gel (ISCO, 330 g column, ethyl acetate/hexanes (25% to 100%) to yield yellow solid (14.65 g, 77%). Experimental Data; LCMS 50% H₂O, Retention time 3.28 min., C₁₈H₂₅FN₃O₆, MW 379.4; Found APCl⁺ 280.2 (M+1-Boc) and APCl⁻ 379.2 (M+); ¹H NMR (CDCl₃) δ 8.88 (d, 1H), 8.39 (m, 1H, br, 1H), 8.05-8.03 (m, 1H), 6.88 (d, 1H), 4.11 (m, br, 2H) 3.89 (s, 3H), 3.75 (m, br, 1H), 3.05 (m, br, 2H), 2.04 (m, br, 2H), 1.57 (m, 2H), 1.47 (s, 9H).

### Preparation of tert-butyl 4-{[2-amino-4-(methoxycarbonyl)phenyl]amino}piperidine-1-carboxylate

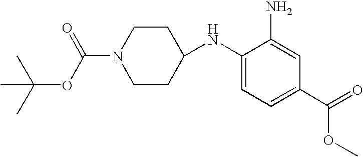

tert-butyl 4-{[4-(methoxycarbonyl)-2-nitrophenyl]amino}piperidine-1-carboxylate (14.65 g, 38.61 mmole) was reduced using 3 g of RaNi in tetrahydrofuran (THF, 150 ml) and concentrated to yield the desired product (foam, 13.37 g, 99%). Experimental Data; LCMS 50% H₂O, Retention time 2.06 min., C₁₈H₂₇N₃O₄, MW 349.4; Found APCl⁺ 350.2 (M+1), 250.2 (M+1-Boc); ¹H NMR (CDCl₃) δ 7.56 (d, br, 1H), 7.47 (s, br, 1H), 6.68 (d, 1H), 4.03 (br, 2H), 3.84 (s, 3H), 3.74 (m, THF), 3.49 (br, 1H), 2.93 (br, 2H), 2.04 (br, 2H), 1.84, (m, THF), 1.45 (m, 11H).

### Preparation of methyl 1-[1-(tert-butoxycarbonyl)piperidin-4-yl]-2-oxo-2,3-dihydro-1H-benzimidazole-5-carboxylate

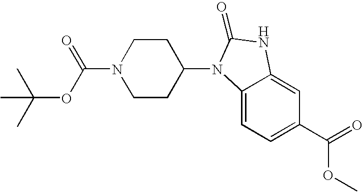

Treated a solution of tert-butyl 4-{[2-amino-4-(methoxycarbonyl)phenyl]amino}piperidine-1-carboxylate (13.3 g, 38.1 mmole) in DCM (375 ml) with 1,1′-carbonyldiimidazole (CDl, 9.27 g, 57.2 mmole) and stirred overnight at room temperature. The reaction was diluted with DCM and washed with water, 0.5N HCl (2×), saturated NaHCO₃, and brine. The organics were dried over Na₂SO₄, filtered, and concentrated to yield a foam (14.78 g, 103%). Experimental Data; LCMS 50% H₂O, Retention time 1.83 min., C₁₉H₂₅N₃O₅, MW 375.4; Found APCl⁺ 276.2 (M+1-Boc) and APCl⁻ 374.3 (M−1); ¹H NMR (CDCl₃) δ 9.29 (s, br, 1H), 7.83-7.78 (m, 2H), 7.14 (d, 1H), 4.55-4.05 (m, 1H), 4.35 (br, 2H), 3.91 (s, 3H), 2.90 (m, 2H), 2.33 (m, 2H), 1.84 (br, 2H), 1.51 (s, 9H).

### Preparation of 1-[1-(tert-butoxycarbonyl)piperidin-4-yl]-2-oxo-2,3-dihydro-1H-benzimidazole-5-carboxylic acid

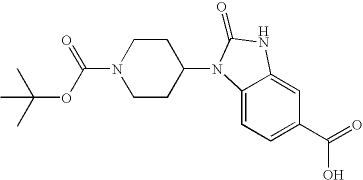

Treated methyl 1-[1-(tert-butoxycarbonyl)piperidin-4-yl]-2-oxo-2,3-dihydro-1H-benzimidazole-5-carboxylate (7.67 g, 20.42 mmole) in MeOH (100 ml) with 1N NaOH (50.8 ml, 50.8 mmole) and stirred overnight at −60° C. After concentrating, the residue was taken up in water and treated with 1N HCl till pH ˜2 forming a precipitate. Collected solid by filtration and washed with water. Solid was dried in a vacuum oven at −45° C. overnight to yield a white solid (6.31 g, 86%). Experimental Data; LCMS 90% H₂O, Retention time 3.17 min., C₁₈H₂₃N₃O₅, MW 361.4; Found APCl⁺ 262.2 (M+1-Boc) and APCl⁻ 360.3 (M−1); ¹H NMR (DMSO-d₆) δ 12.58 (s, 1H), 11.06 (s, 1H), 7.61-7.59, (m, 1H), 7.45 (s, 1H), 7.25 (d, 1H), 4.32 (br, 1H), 4.04 (d, br, 2H), 2.82 (br, 2H), 2.19-2.09 (m, 2H), 1.65 (br, 2H), 1.38 (s, 9H).

### Preparation of tert-butyl 4-{5-[(methylamino)carbonyl]-2-oxo-2,3-dihydro-1H-benzimidazol-1-yl}piperidine-1-carboxylate

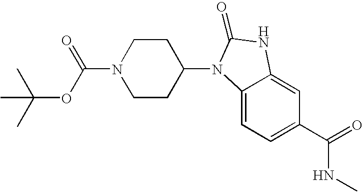

Mixture of 1-[1-(tert-butoxycarbonyl)piperidin-4-yl]-2-oxo-2,3-dihydro-1H-benzimidazole-5-carboxylic acid (6.28 g, 17.37 mmole), N-(3-Dimethylaminopropyl)-N′-ethylcarbodiimide (EDC, 5.10 g, 26.1 mmole) and 1-Hydroxybenzotriazole (HOBt, 3.90 g, 26.1 mmole) in dichloromethane (DCM, 175 ml) at 0° C. was stirred for 20 minutes. To the mixture was added methylamine (2M in tetrahydrofuran, 43.4 ml, 86.8 mmole). This reaction mixture at 0° C. was allowed to warm to room temperature and stirred for 4 hours. The reaction mixture was further diluted with DCM and the resultant organic layer was washed with saturated NaHCO₃ (2×) and brine. The organic layer was dried over Na₂SO₄, filtered, and the filtrate was concentrated to yield foam. The foam was purified by chromatography on silica gel (Isco, 120 g column, MeOH/DCM (0 to 5%)) to yield a solid (5.12 g, 79%). Experimental Data; LCMS 90% H₂O, Retention time 3.05 min., C₁₉H₂₆N₄O₄, MW 374.4; Found APCl⁺ 275.2 (M+1-Boc) and APCl⁻ 373.3 (M−1); ¹H NMR (CDCl₃) δ 9.69 (s, 1H), 7.61 (d, 1H), 7.47-7.44 (m, 1H), 7.11 (d, 1H), 6.30 (br, 1H), 4.49-4.41 (m, br, 1H), 4.32 (m, br, 2H), 3.02 (s, 3H), 2.89-2.83 (m, 2H), 2.35-2.24 (m, 2H), 1.82 (m, br, 2H), 1.50 (s, 9H).

### Preparation of N-methyl-2-oxo-1-piperidin-4-yl-2,3-dihydro-1H-benzimidazole-5-carboxamide; trifluoroacetic acid salt

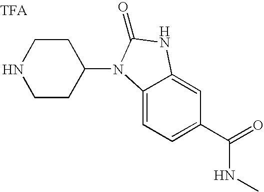

Was treated tert-butyl 4-{5-[(methylamino)carbonyl]-2-oxo-2,3-dihydro-1H-benzimidazol-1-yl}piperidine-1-carboxylate (5.72 g, 15.27 mmole) with 20% trifluoracetic acid (TFA)/DCM (250 ml), and the reaction mixture was stirred for 2 hours at room temperature. The reaction mixture was subsequently concentrated and the residue was taken up in DCM and evaporated under reduced pressure repeatedly (3×) to yield a off-white solid (10.2 g, 3.5 equivalents of TFA). Experimental Data; LCMS 98% H₂O, Retention time 1.99 min., Parent C₁₄H₁₈N₄O₂, MW 274.3; Found APCl⁺ 275.2 (M+1) and APCl⁻ 273.2 (M−1); ¹H NMR (CD₃OD) δ 7.59-7.56 (m, 1H), 7.52 (d, 1H), 7.28 (d, 1H), 4.56-4.50 (m, br, 1H), 3.56-3.53 (m, br, 2H), 3.22-3.15 (m, br, 1H), 2.88 (s, 3H), 2.72-2.68 (m, 2H), 2.03 (m, br, 2H).

### EXAMPLE NO. 1

### 1-{1-[2-(4-chlorophenyl)-2-oxoethyl]piperidin-4-yl}N-methyl-2-oxo-2,3-dihydro-1H-benzimidazole-5-carboxamide

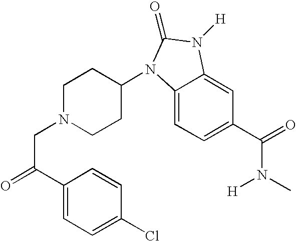

Ice cold suspension of N-methyl-2-oxo-1-piperidin-4-yl-2,3-dihydro-1H-benzimidazole-5-carboxamide trifluoroacetic acid salt (10.23 g, assumed to be 15.27 mmole) in DCM (175 ml) was treated with triethyl amine (21.28 ml, 152.69 mmole) and stirred for 5 minutes. The reaction mixture was treated with 4-chlorophenacyl bromide (3.57 g, 15.27 mmole) and was stirred at room temperature for 2 hours. The reaction mixture was treated with an additional 4-chlorophenacyl bromide (0.35 g. 1.5 mmole) and stirred for 0.5 hour. The reaction mixture was diluted with DCM and washed with saturated NaHCO₃ (2×). The organic layer was dried over Na₂SO₄, filtered, and the filtrate was concentrated to amorphous material.

The material was purified by chromatography on silica gel (ISCO, 120 g column, MeOH/DCM (1 to 10%)) to give foam (2.10 g, 32%). Experimental Data; LCMS 90% H₂O, Retention time 2.40 min., C₂₂H₂₃ClN₄O₃, MW 426.1; Found APCl⁺ 427.1 (M+1) and APCl⁻ 425.2 (M−1); ¹H NMR (DMSO-d₆) δ 11.03 (s, 1H), 8.28-8.25 (m, 1H), 7.98 (m, 2H), 7.58-7.47 (m, 3H), 7.41 (s, 1H), 7.20 (d, 1H), 4.11 (br, 1H), 3.85 (s, 2H), 2.97 (br, 2H), 2.71 (d, 3H), 2.33-2.24 (m, 4H), 1.61 (m, 2H).

### EXAMPLE NO. 2

### 1-{2-[4-chlorophenyl)-2-oxoethyl]-4-(5-[(mothylamino)carbonyl]-2-oxo-2,3-dihydro-1H-benzimidazol-1-yl}piperidinium chloride

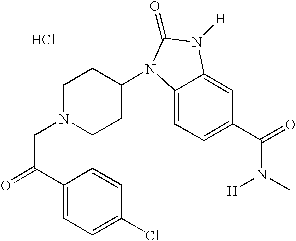

Treated 1-(1-[2-(4-chlorophenyl)-2-oxoethyl]piperidin-4-yl)-N-methyl-2-oxo-2,3-dihydro-1H-benzimidazole-5-carboxamide (2.099 (4.90 mmol) in DCM (150 ml) with HCl(g) for 10 minutes. The reaction mixture was sealed and stirred at room temperature for 2 hours and subsequently concentrated. The residue was treated with 11 ml of MeOH (anhydrous) and 25 ml of diethyl ether. The mixture was placed in the refrigerator for 1 hour. The solid was collected and washed with cold diethyl ether and then dried in a vacuum oven at 40-45° C. overnight to yield a white solid (1.59 g, 70%). Experimental Data; LCMS 90% H₂O, Retention time 2.41 min., Parent C₂₂H₂₃ClN₄O₃, MW 426.1; Found APCl⁺ 427.1 (M+1) and APCl⁻ 425.2 (M−1); ¹H NMR (CD₃OD) δ 8.02 (d, 2H), 7.61-7.58 (m, 3H), 7.53 (d, 1H), 7.38 (d, 1H), 4.95 (s, 2H), 4.65-4.59 (m, 1H), 3.74 (br, 2H), 3.36 (br, 2H), 2.98-2.88 (m, 5H), 2.08 (br, 2H). CHN Calc. (1.19 HCl): C, 56.19; H, 5.18; N, 11.91; total Cl, 16.51 Found: C, 56.56; H, 5.13; N, 11.94; total Cl, 16.8

### Preparation of methyl 2,4-difluoro-5-nitrobenzoate (Chem. Abstr. Reg. No. 125568-71-0)

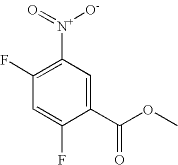

2,4-Difluoro-5-nitrobenzoic acid (33.0 g, 162 mmol) was dissolved in 400 mL anhydrous methanol under an argon atmosphere, added 4 mL concentrated sulfuric acid, then heated to reflux for 15 hours. The solution was cooled and concentrated in vacuo. Redissolved in 400 mL diethyl ether, washed with sat. sodium bicarbonate (3×200 mL), brine, dried with magnesium sulfate, filtered and concentrated to yield title compound. (32.6 g, 92%) APCl⁻ 217.0; Anal. HPLC Retention time=15.7 minutes (>99% pure).

### Preparation of tert-butyl 4-{[5-fluoro-4-(methoxycarbonyl)-2-nitrophenyl]amino}piperidine-1-carboxylate

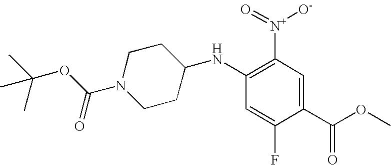

methyl 2,4-difluoro-5-nitrobenzoate (32.6 g, 150 mmol) was dissolved in 100 mL N,N-dimethylformamide, added 4-amino-1-Boc-piperidine (30.7 g, 153 mmol), washed sides of flask with 100 mL N,N-dimethylformamide, added N,N-diisopropylethylamine and stirred reaction at ambient temperature for 3 days. The solution was diluted with 500 mL diethyl ether, washed with water (2×250 mL), 0.5M hydrogen chloride (2×250 mL), brine (250 mL), dried with magnesium sulfate, filtered and concentrated to yield title compound slightly wet with diethyl ether. (62.4 g, quant yield) APCl⁻ 396.2; Anal. HPLC Retention time=20.2 minutes.

### Preparation of tert-butyl 4-{[2-amino-5-fluoro-4-(methoxycarbonyl)phenyl]amino}piperidine-1-carboxylate

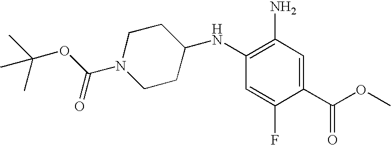

tert-butyl 4-{[5-fluoro-4-(methoxycarbonyl)-2-nitrophenyl]amino}piperidine-1-carboxylate (59.6 g, 162.4 mmol) was dissolved in tetrahydrofuran (1000 mL), added RaNi and shaken for 0.53 hours. The solution was filtered and concentrated in vacuo to yield title compound. APCl⁺ 268.0; Anal. HPLC Retention time=14.8 minutes.

### Preparation of methyl 1-[1-(tert-butoxycarbonyl)piperidin-4-yl]-6-fluoro-2-oxo-2,3-dihydro-1H-benzimidazole-5-carboxylate

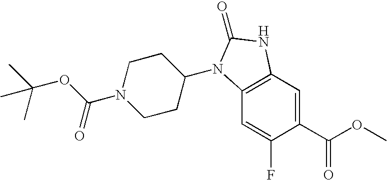

tert-butyl 4-{[2-amino-5-fluoro-4-(methoxycarbonyl)phenyl]amino}piperidine-1-carboxylate (56.8 g, 155 mmol) was dissolved in 400 mL tetrahydrofuran at 0° C., added 1,1′-carbonyldiimidazole (26.3 g, 162 mmol) in small portions, stirred at 0° C. for 15 minutes, then removed cooling bath. The reaction mixture was stirred at ambient temperature for 3 hours. The solution was diluted with 400 mL ethyl acetate, washed organics with 0.5M hydrogen chloride (3×250 mL) and brine (2×200 mL). Filter off a suspended solid in the organic layer. The filtrates were then dried with magnesium sulfate, filtered and concentrated in vacuo. The resultant material was re-suspended material in about 300 mL ethyl acetate, this suspension was stirred at 70° C. for 30 minutes then removed from heat. Allowed to cool to room temperature, then placed the flask in the freezer for 2 hours. The solids were filtered, washing with several portions of cold ethyl acetate, followed by several portions of hexanes. Solids were dried in vacuum oven to yield title compound. (35.7 g, 59%) APCl⁻ 392.2; Anal. HPLC Retention time=16.7 minutes (>99% pure).

### Preparation of 1-[1-(tert-butoxycarbonyl)piperidin-4-yl]-6-fluoro-2-oxo-2,3-dihydro-1H-benzimidazole-5-carboxylic acid

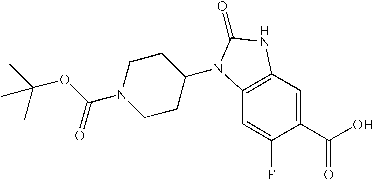

methyl 1-[1-(tert-butoxycarbonyl)piperidin-4-yl]-6-fluoro-2-oxo-2,3-dihydro-1H-benzimidazole-5-carboxylate (29.1 g, 74.0 mmol) was suspended in 150 mL methanol, then added 2M sodium hydroxide. The reaction mixture was stirred at 60° C. for 2 hours. The reaction was first cooled to ambient temperature, diluted with 1000 mL tetrahydrofuran, acidified by adding 1M aq. hydrogen chloride (350 mL) dropwise via an addition funnel. The resultant solution was washed with brine (2×200 mL), dried with magnesium sulfate. The solution was filtered and concentrated in vacuo overnight to yield title compound. (27.9 g, 99%) APCl⁻ 378.1; Anal. HPLC Retention time=14.9 minutes (>99% pure).

### Preparation of tert-butyl 4-{6-fluoro-5-[(methylamino)carbonyl]-2-oxo-2,3-dihydro-1H-benzimidazol-1-yl}piperidine-1-carboxylate

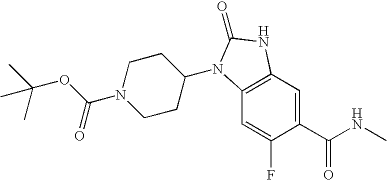

1-[1-(tert-butoxycarbonyl)piperidin-4-yl]-6-fluoro-2-oxo-2,3-dihydro-1H-benzimidazole-5-carboxylic acid (27.9 g, 73.5 mmol) was suspended in 500 mL anhydrous dichloromethane, added N-(3-dimethylaminopropyl)-N′-ethylcarbodiimide hydrochloride (21 g, 110 mmol) and 1-hydroxybenzotriazole hydrate (17 g, 110 mmol), then stirred at ambient temperature for 20 minutes. Cooled the solution in an ice bath and added 2M methylamine in tetrahydrofuran slowly. After addition the cooling bath was removed. The reaction mixture was stirred at ambient temperature overnight. The solution was diluted with 300 mL dichloromethane, washed with 1M aqueous hydrogen chloride (3×250 mL), sat. sodium bicarbonate (3×250 mL), brine, dried with magnesium sulfate, filtered and concentrated. The residue was dissolved in 250 mL hot ethyl acetate (˜60° C.), then let sit at ambient temperature overnight. Solids were filtered off the next day, washing with cold ethyl acetate, followed by hexanes to yield title compound. (22.7 g, 79%) APCl⁻ 391.2; Anal. HPLC Retention time=14.8 minutes (>99% pure).

### Preparation of 4-{6-fluoro-5-[(methylamino)carbonyl]-2-oxo-2,3-dihydro-1H-benzimidazol-1-yl}piperidinium trifluoroacetate

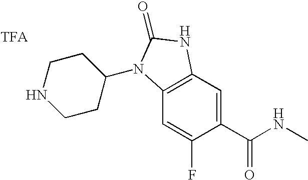

tert-butyl 4-{6-fluoro-5-[(methylamino)carbonyl]-2-oxo-2,3-dihydro-1H-benzimidazol-1-yl}piperidine-1-carboxylate (22.7 g, 57.8 mmol) was dissolved in 300 mL dichloromethane, placed in an ice bath, then added 60 mL of trifluoroacetic acid. After 5 minutes the cooling bath was removed and the reaction stirred at ambient temperature for 2.5 hours. The reaction mixture was concentrated in vacuo, co-concentrated with 50 mL of chloroform 3 times to yield the title compound containing approximately 3 equivalents of trifluoroacetic acid. (40 g, quant. yield) APCl⁺ 293.3; Anal. HPLC Retention time=9.8 minutes (>99% pure).

### EXAMPLE NO. 3

### 1-{-[2-(4-chlorophenyl)-2-oxoethyl]piperidin-4-yl}-6-fluoro-N-methyl-2-oxo-2,3-dihydro-1H-benzimidazole-5-carboxamide

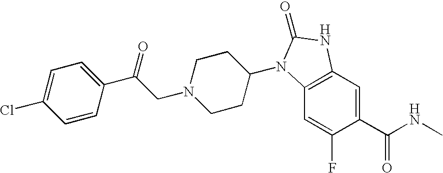

4-{6-fluoro-5-[(methylamino)carbonyl]-2-oxo-2,3-dihydro-1H-benzimidazol-1-yl}piperidinium trifluoroacetate (0.82 g, 2.8 mmol) was dissolved in 200 mL anhydrous N,N-dimethylformamide under argon, cooled to −15° C., then added triethylamine (1.56 mL, 11.2 mmol) dropwise via an addition funnel. Stirred for 5 minutes, then added 4′-chlorophenacyl bromide (0.62 g, 2.66 mmol) dropwise over 15 minutes via an addition funnel (dissolved in 4 mL N,N-dimethylformamide). The reaction mixture was stirred at −15° C. for a total of 1 hour from beginning of bromide addition. The flask was moved to an ice/water bath at 0° C. to which was added 385 mL of 5% aqueous sodium bicarbonate (% w/w) dropwise via addition funnel over a period of about 20 minutes. The resultant suspension was then poured into 1500 mL ice cold water, formed slurry for 10 minutes. The solid was filtered off and washed with several portions of water. Subsequently, the solid was dried over vacuum filter flask to yield the title compound. (1.16 g, 93%) APCl⁺ 445.1, 447.1 (Cl pattern); Anal. HPLC Retention time=12.8 minutes (97.7% pure). ¹H NMR (DMSO-d₆) δ 11.0 (s, 1H), 8.00 (m, 2H), 7.94 (m, 1H), 7.55 (m, 2H), 7.16 (m, 2H), 4.08 (m, 1H), 3.86 (s, 2H), 2.95 (m, 2H), 2.71 (d, 3H), 2.35-2.23 (m, 4H), 1.59 (m, 2H).

### EXAMPLE NO. 4

### 1-{1-[2-(4-chlorophenyl)-2-hydroxyethyl]piperidin-4-yl}-6-fluoro-N-methyl-2-oxo-2,3-dihydro-1H-benzimidazole-5-carboxamide

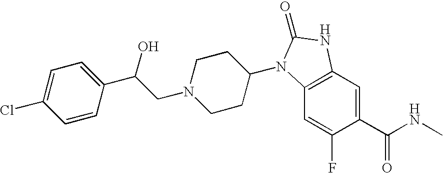

1-{1-[2-(4-chlorophenyl)-2-oxoethyl]piperidin-4-yl}-6-fluoro-N-methyl-2-oxo-2,3-dihydro-1H-benzimidazole-5-carboxamide (0.41 g, 0.87 mmol) was dissolved in 15 mL ethanol at ambient temperature, added sodium borohydride (33 mg, 0.87 mmol) in one portion, then stirred at ambient temperature for 3 hours. Water (80 mL) was slowly added to the solution. A white solid precipitates. Vacuum filtered solids using filter paper, then air dried over vacuum flask overnight to yield title compound. (0.39 g, quant) APCl⁺ 447.1, 449.1 (Cl pattern); Anal. HPLC Retention time=12.8 minutes. ¹H NMR (DMSO-d₆) δ 11.0 (s, 1H), 7.93 (m, 1H), 7.34 (m, 4H), 7.21 (m, 1H), 7.17 (m, 1H), 5.07 (d, 1H), 4.67 (m, 1H), 4.08 (m, 1H), 3.00 (m, 2H), 2.72 (d, 3H), 2.4-2.13 (m, 4H), 1.57 (m, 2H).

### EXAMPLE NO. 5

### 1-[2-(4-chlorophenyl)-2-oxoethyl]-4-[6-fluoro-5-(methoxycarbonyl)-2-oxo-2,3-dihydro-1H-benzimidazol-1-yl]piperidinium chloride

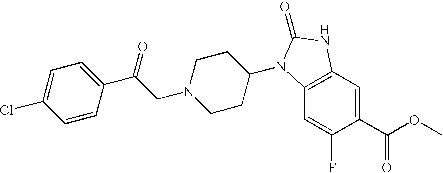

methyl 1-[1-(tert-butoxycarbonyl)piperidin-4-yl]-6-fluoro-2-oxo-2,3-dihydro-1H-benzimidazole-5-carboxylate (0.82 g, 2.08 mmol) was stirred in 20 mL dichloromethane and 10 mL trifluoroacetic acid at ambient temperature for 1 hour which was subsequently concentrated in vacuo. The concentrated reaction mixture was suspended in 20 mL dichloromethane, cooled to 0° C. To this solution, was added triethylamine (2.9 mL, 20.8 mmol) followed by 4′-chlorophenacyl bromide (0.49 g, 2.08 mmol). The reaction mixture was warmed to ambient temperature overnight. The solution was taken up in ethyl acetate, washed with water, brine, dried with magnesium sulfate, filtered and concentrated in vacuo. The desired product was purified on a silica gel column eluting with a gradient of 25-100% ethyl acetate in hexanes. The appropriate fractions were combined and concentrated to an orange film. This was purified again by preparatory HPLC (10-60% acetonitrile in water; 0.1% trifluoroacetic acid). The pure fractions were combined and concentrated, converted to the hydrochloride salt with Amberlite IRA-400 (Cl) ion exchange resin, then lyophilized to yield title compound. (69 mg, 7%) APCl⁺ 446.1, 448.0 (Cl pattern); Anal. HPLC Retention time=14.0 minutes (>99% pure).

### EXAMPLE NO. 6

### 1-[2-(4-chlorophenyl)-2-hydroxyethyl]-4-[6-fluoro-5-(methoxycarbonyl)-2-oxo-2,3-dihydro-1H-benzimidazol-1-yl]piperidinium chloride

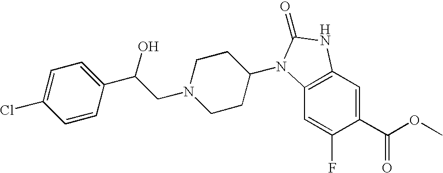

1-[2-(4-chlorophenyl)-2-oxoethyl]-4-[6-fluoro-5-(methoxycarbonyl)-2-oxo-2,3-dihydro-1H-benzimidazol-1-yl]piperidinium chloride (0.37 g, 0.83 mmol) was suspended in 10 mL ethanol under an argon atmosphere, added sodium borohydride (63 mg, 1.7 mmol) and stirred the reaction at ambient temperature for 2 hours, diluted with ethyl acetate, washed with water, brine, dried with magnesium sulfate, filtered and concentrated in vacuo. The concentrated material was purified by preparatory HPLC. Collected pure fractions were combined and concentrated, converted to the hydrochloride salt using Amberlite IRA-400 (Cl) ion exchange resin. The solution was lyophilized to yield title compound. (0.32 g, 86%) APCl⁻ 446.1, 448.1 (Cl pattern); Anal. HPLC Retention time=13.9 minutes (>99% pure).

### EXAMPLE NO. 7

### 4(5-carboxy-6-fluoro-2-oxo-2,3-dihydro-1H-benzimidazol-1-yl)-1-[2-(4-chlorophenyl)-2-hydroxyethyl]piperidinium chloride

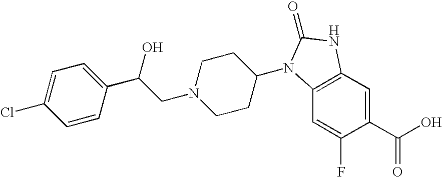

1-[2-(4-chlorophenyl)-2-hydroxyethyl]-4-[6-fluoro-5-(methoxycarbonyl)-2-oxo-2,3-dihydro-1H-benzimidazol-1-yl]piperidinium chloride (0.25 g, 0.56 mmol) was dissolved in 5 mL methanol, added 1M sodium hydroxide (3.3 mL, 3.3 mmol) then heated to 60° C. The solution was cooled, acidified with 1M aq. hydrogen chloride, extracted with ethyl acetate (added tetrahydrofuran to dissolve solids), dried with magnesium sulfate, filtered and concentrated in vacuo. The reaction mixture was purified by preparatory HPLC. Collected desired fractions were combined, concentrated and converted to hydrochloride salt using Amberlite IRA-400 (Cl) ion exchange resin. The concentrated product was lyophilized to yield the title compound. (125 mg, 52%) APCl⁺ 434.1, 436.0 (Cl pattern); Anal. HPLC Retention time=12.9 minutes (99% pure).

### EXAMPLE NO. 8

### 1-(1-[2-(4 chlorophenyl)-2-fluoroethyl]piperidin-4-yl)-6-fluoro-N-methyl-2-oxo-2,3-dihydro-1H-benzimidazole-5-carboxamide

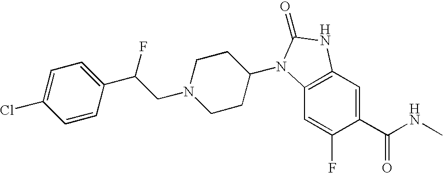

1-{1-[2-(4-chlorophenyl)-2-hydroxyethyl]piperidin-4-yl}-6-fluoro-N-methyl-2-oxo-2,3-dihydro-1H-benzimidazole-5-carboxamide (0.34 g, 0.76 mmol) was dissolved in 10 mL anhydrous dichloromethane under an argon atmosphere, cooled to −78° C., then added bis(2-methoxyethyl)aminosulfur trifluoride (2.2 mL, 6.1 mmol) dropwise. The reaction mixture was stirred at −78° C. for 3 hours. The reaction mixture was quenched with saturated sodium bicarbonate, extracted with ethyl acetate, washed organics with brine, dried with magnesium sulfate, filtered and concentrated. The reaction mixture was purified by preparatory HPLC. Combined and lyophilized pure fractions to yield title compound. (43 mg, 12%) APCl⁺ 449.2, 451.1 (Cl pattern); Anal. HPLC Retention time=13.5 minutes (97% pure).

### Preparation of 2-bromo-1-(4-ethylphenyl)ethanone (Chem. Abstr. Reg. No. 2632-14-6)

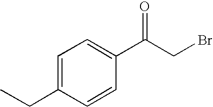

4′-ethylacetophenone (9 mL, 60 mmol) was dissolved in 100 mL anhydrous tetrahydrofuran, cooled to 0° C., then added phenyltrimethylammonium tribromide (22.6 g, 60 mmol) in portions. A precipitate slowly forms. The reaction mixture was stirred at 0° C. for 1 hour, then at ambient temperature for 20 minutes. The concentrated reaction mixture was diluted with 200 mL diethyl ether, washed with water (3×150 mL), brine, and dried with magnesium sulfate, filtered. Thw filtrate was concentrated to yield the title compound. (15.9 g, quant.) MS: 227, 229.03 (Br pattern); Anal. HPLC Retention time=18.6 minutes (>99% pure).

### EXAMPLE NO. 9

### 1-[2-(4-ethylphenyl)-2-oxoethyl]-4-{6-fluoro-5-[(methylamino)carbonyl]-2-oxo-2,3-dihydro-1H-benzimidazol-1-yl}piperidinium chloride

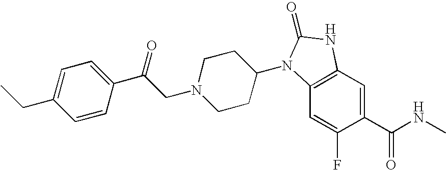

4-{6-fluoro-5-[(methylamino)carbonyl]-2-oxo-2,3-dihydro-1H-benzimidazol-1-yl}piperidinium trifluoroacetate (20 g, 31 mmol) was dissolved in 100 mL N,N-dimethylformamide, added triethylamine (25.9 mL, 186 mmol) followed by the addition of 2-bromo-1-(4-ethylphenyl)ethanone (11 g, 37 mmol) which was dissolved in 20 mL N,N-dimethylformamide. The reaction mixture was stirred at ambient temperature for 1 hour. An aqueous solution of 5% sodium bicarbonate (300 ml) was added slowly. The desired compound was extracted with dichloromethane (400 mL), and the organic layer was washed with water and brine, dried with magnesium sulfate, filtered. The filtrate was concentrated in vacuo. The desired product was purified by column chromatography eluting with 100% ethyl acetate for, then 0-30% methanol in ethyl acetate. Desired spot eluted with impurities and some of the orange color. All fractions containing product were concentrated in vacuo to a light orange solid. This was further purified by column chromatography eluting with a gradient of 1-40% methanol in ethyl acetate. A set of fractions were combined that look pure by TLC and concentrated in vacuo. Suspended were solids in 100 mL ethyl acetate at 50° C. and was cooled to ambient temperature. Solids were filtered off washed with cold ethyl acetate. The resultant solid was dissolved in 50 mL methanol and 100 mL dichloromethane, and subsequently cooled to 0° C. through which hydrogen chloride gas was bubbled for 1 minute. The solution was concentrated together with 50 mL methanol, and subsequently re-suspended in 50 mL methanol, suspended at 40° C. for 5 minutes, to which was added 300 mL of diethyl ether. Solids were filtered off to yield the title compound. (5.92 g, 42%) APCl⁺ 439.21; Anal. HPLC Retention time=13.5 minutes (98% pure). ¹H NMR (DMSO-d₆) δ 11.2 (s, 1H), 10.2 (s, 1H), 8.00 (m, 1H), 7.91 (d, 2H), 7.52 (d, 1H), 7.43 (d, 2H), 7.23 (d, 1H), 5.04 (m, 2H), 4.57 (m, 1H), 3.64 (m, 2H), 3.3 (m, 2H), 2.8-2.63 (m, 7H), 1.92 (m, 2H), 1.18 (t, 3H).

### EXAMPLE NO. 10

### 1-{1-[2-(4-ethylphenyl)-2-hydroxyethyl]piperidin-4-yl}-6-fluoro-N-methyl-2-oxo-2,3-dihydro-1H-benzimidazole-5-carboxamide

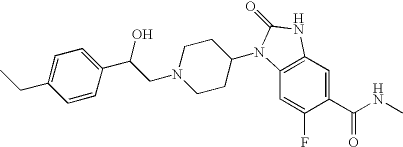

1-{1-[2-(4-ethylphenyl)-2-oxoethyl]piperidin-4-yl}-6-fluoro-N-methyl-2-oxo-2,3-dihydro-1H-benzimidazole-5-carboxamide (3.08 g, 7.02 mmol) was suspended in 50 mL ethanol, added sodium borohydride (0.27 g, 7.02 mmol) and stirred the reaction mixture at ambient temperature for 1.5 hours. Water (400 mL) was slowly added to the stirring solution. A precipitate formation was noted. The suspension was stirred at ambient temperature for 2 hours, then filtered off solids. The solids were washed with water to yield the title compound (2.4 g, 78%) APCl⁺, 441.20; Anal. HPLC Retention time=13.6 minutes (97% pure). ¹H NMR (DMSO-d₆) δ 11.0 (s, 1H), 7.95 (m, 1H), 7.23 (m, 3H), 7.19 (d, 1H), 7.13 (d, 2H), 4.89 (m, 1H), 4.66 (m, 1H), 4.10 (m, 1H), 3.05 (m, 2H), 2.74 (d, 3H), 2.55 (q, 2H), 2.4-2.15 (m, 6H), 1.59 (m, 2H), 1.14 (t, 3H).

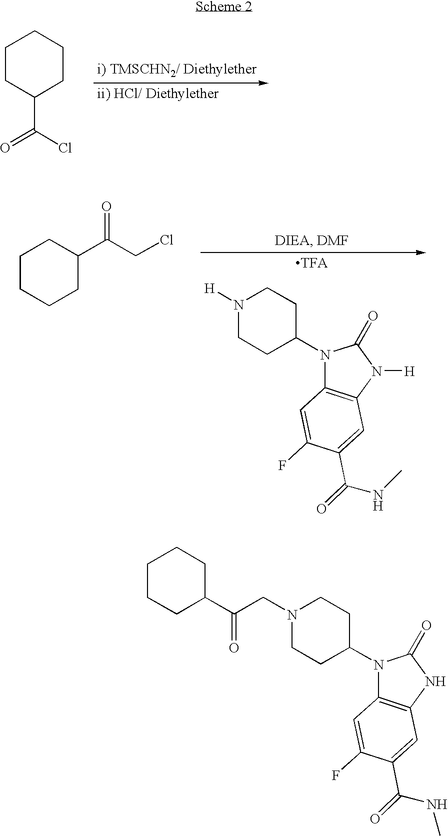

### Preparation of 2-Chloro-1-cyclohexylethanone (Chem. Abstr. Reg. No. 1892-09-7)

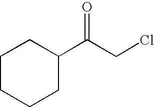

To a solution containing trimethylsilyldiazomethane in ether (1.70 g, 15.0 mmol 7.50 mL) and triethylamine (15.0 mmol) at −25° C., was dropwise added a solution of commercially available cyclohexanecarboxylic acid chloride (1.00 g, 6.80 mmol) in anhydrous ether (25 mL). While agitated the reaction mixture was allowed to warm to room temperature for 5 h. The reaction mixture was concentrated to obtain the desired trimethylsilyl intermediate. The intermediate was directly added to 1N HCl in ether at 0° C. and warmed to room temperature for 5 h. The reaction mixture was then evaporated under reduced pressure. The residue was diluted with EtOAc (50.0 mL) and washed with 1M NaHCO₃ (10 mL), water (15 mL×2) and brine. The organic layer was dried over MgSO₄ and filtered. The filtrate was then concentrated under reduced pressure to give a pale-yellow liquid. The crude material was used to the next reaction without further purification (0.928 g, yield, 85%). ¹H NMR (DMSO-d₆) δ 4.11 (s, 2H), 1.78 (m, 4H), 1.25 (m, 6H).

### EXAMPLE NO. 11

### 1-[1-(2-cyclohexyl-2-oxoethyl)piperidin-4-yl]-6-fluoro-N-methyl-2-oxo-2,3-dihydro-1H-benzimidazole-5-carboxamide

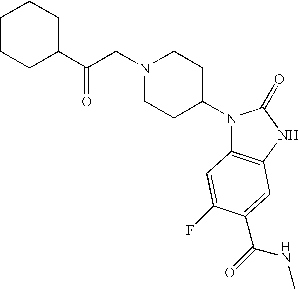

To a solution of 4-{6-fluoro-5-[(methylamino)carbonyl]-2-oxo-2,3-dihydro-1H-benzimidazol-1-yl}piperidinium trifluoroacetate in DMF (2 mL), 2-Chloro-1-cyclohexylethanone (0.1 g, 0.61 mmol) and TEA (0.7 mL, 5.08 mmol) were added sequentially at room temperature, and the reaction mixture was stirred for 4 h. The reaction mixture was diluted with water (15 mL) and extracted with EtOAc (2×10 mL). The organic layer was washed with brine solution (15 mL), dried over anhydrous sodium sulfate and concentrated to obtain the crude product. The crude product was purified by preparative HPLC method to obtain the pure product as off white solid (45 mg, 21.4%). IR (cm⁻¹): 2927, 2680, 1685, 1384 and 1172. ¹HNMR (DMSO): δ 11.2(s, 1H), 9.65-9.9 (broad s, 1H), 8.04 (m, 1H), 7.45 (d, 1H), 7.26 (d, 1H), 4.4-4.72 (m, 3H), 3.12-3.3 (m, 2H), 2.56-2.86 (m, 4H), 1.5-2.0 (m, 6H) and 1.1-1.4 (m, 4H). Mass: (M+1) 417 calculated for C₂₂H₂₉FN₄O₃.

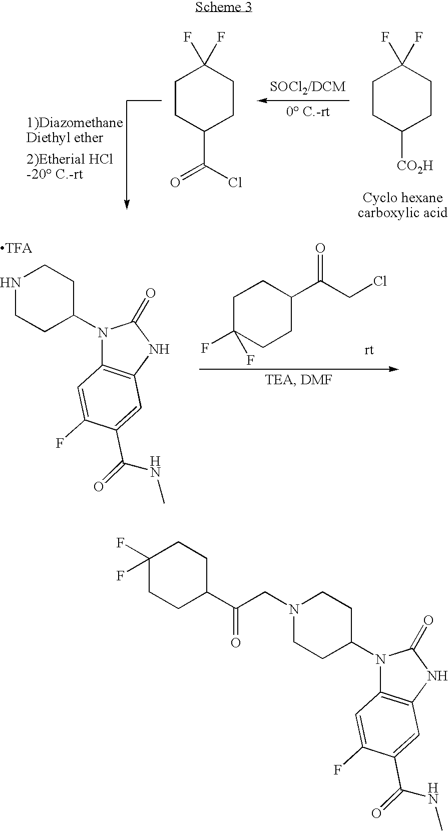

### Preparation of 4,4-Di-fluoro-cyclohexanecarbonyl chloride

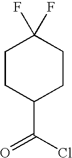

A solution of 4,4-Di-fluoro-cyclohexanecarboxylic acid (0.43 g, 3.028 mmol) (Chem. Abstr. Reg. No. 122665-97-8) in dry DCM (5 mL) was cooled to 0° C., SOCl₂ (0.329 mL, 4.54 mmol) was added. The reaction mixture was allowed to rt, maintained for 3 h and then was concentrated to obtain the crude acid halide. The crude was directly used in the next step without any purification.

### Preparation of 2-chloro-1-(4,4-difluorocyclohexyl)ethanone

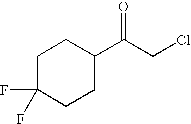

To a solution containing trimethylsilyidiazomethane in ether (1.70 g, 15.0 mmol 7.50 mL) and triethylamine (15.0 mmol) at −25° C., was dropwise added a solution of commercially available cyclohexanecarboxylic acid chloride (1.00 g, 6.80 mmol) in anhydrous ether (25 mL). While agitated the reaction mixture was allowed to warm to room temperature for 5 h. The reaction mixture was concentrated to obtain the desired trimethylsilyl intermediate. The intermediate was directly added to 1N HCl in ether at 0° C. and warmed to room temperature for 5 h. The reaction mixture was then evaporated under reduced pressure. The residue was diluted with EtOAc (50.0 mL) and washed with 1M NaHCO₃ (10 mL), water (15 mL×2) and brine. The organic layer was dried over MgSO₄ and filtered. The filtrate was then concentrated under reduced pressure to give a pale-yellow liquid. The crude material was used to the next reaction without further purification (0.928 g, yield, 85%). ¹H NMR (DMSO-d₆) δ 4.11 (s, 2H), 1.78 (m, 4H), 1.25 (m, 6H).

### EXAMPLE NO. 12

### 1-{1-[2-(4,4-difluorocyclohexyl)-2-oxoethyl]piperidin-4-yl}-6-fluoro-N-methyl-2-oxo-2,3-dihydro-1H-benzimidazole-5-carboxamide

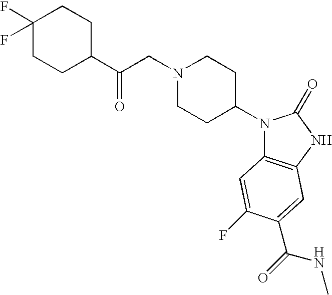

To a solution of 4-{6-fluoro-5-[(methylamino)carbonyl]-2-oxo-2,3-dihydro-1H-benzimidazol-1-yl}piperidinium trifluoroacetate (0.55 g, 1.88 mmol) in dry DMF (5 mL), TEA (2.61 mL, 18.8 mmol) was added followed by the addition of 2-chloro-1-(4,4-difluorocyclohexyl)ethanone (0.394 g, 2.26 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by preparative HPLC method to obtain the pure product as white solid (0.08 g, 10%). IR (cm⁻¹): 3442, 3230, 2937, 1695, 1485, 1394, 1297, 1186 and 1051. ¹HNMR (CDCl₃): δ 9.02-9.22 (s, 1H), 7.8-8.02 (m, 1H), 7.12 (d, 1H), 6.7-6.96 (m, 1H), 4.3-4.52 (m, 1H), 3.32-3.55 (m, 2H), 2.95-3.16 (m, 4H), 2.22-2.65 (m, 4H), 1.5-2.0 (m, 8H), 1.12-1.52 (m, 3H) and 0.8-1.02 (m, 3H). Mass: (M+1) 431 calculated for C₂₃H₃₁FN₄O₃.

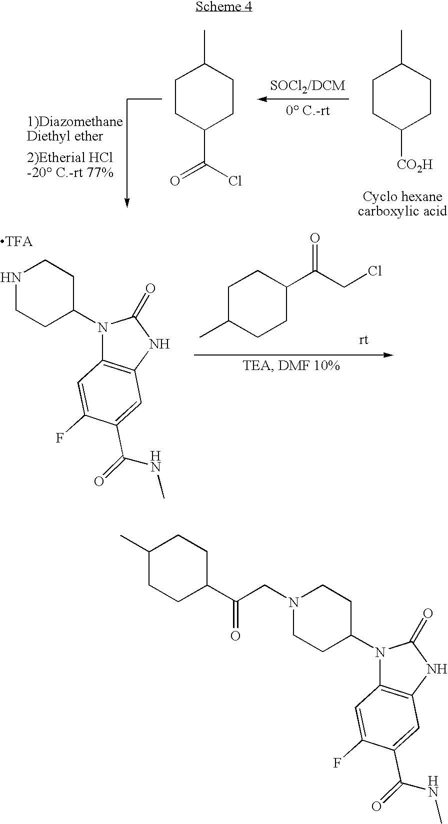

### Preparation of 4-methylcyclohexanecarbonyl chloride

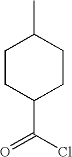

A solution of 4-methylcyclohexanecarboxylic acid (0.43 g, 3.028 mmol) (Chem. Abstr. Reg. No. 4331-54-8) in dry DCM (5 mL) was cooled to 0° C., SOCl₂ (0.329 mL, 4.54 mmol) was added. The reaction mixture was allowed to reach room temperature, maintained for 3 h and then was concentrated to obtain the crude acid halide. The crude was directly used in the next step without any purification.

### Preparation of 2-chloro-1-(4-methylcyclohexyl)ethanone

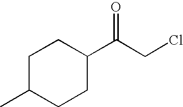

A solution of 4-Methyl-cyclohexanecarbonyl chloride (0.486 g) in diethyl ether (5 mL) was cooled to −20° C., a solution of diazomethane (0.381 g) in diethyl ether (80 mL) was added. The reaction mixture was allowed to 0° C. and maintained for 2 h. The resulted reaction mixture was cooled −20° C., ethereal HCl (8 mL) was added, allowed to rt, maintained for 2 h and concentrated to obtain the crude product (0.4 g, 77%). ¹HNMR (CDCl₃): δ 4.16(s, 2H), 1.72-1.98 (m, 4H), 1.22-1.65 (m, 6H) and 0.85-1.05 (m, 3H).

### EXAMPLE NO. 13

### 6-fluoro-N-methyl-1-{1-[2-(4-methylcyclohexyl)-2-oxoethyl]piperidin-4-yl}-2-oxo-2,3-dihydro-1H-benzimidazole-5-carboxamide

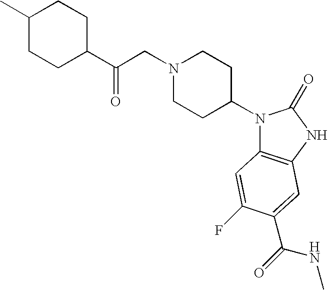

To a solution of product 4-{6-fluoro-5-[(methylamino)carbonyl]-2-oxo-2,3-dihydro-1H-benzimidazol-1-yl}piperidinium trifluoroacetate (0.55 g, 1.88 mmol) in dry DMF (5 mL), TEA (2.61 mL, 18.8 mmol) was added followed by the addition of 2-chloro-1-(4-methylcyclohexyl)ethanone (0.394 g, 2.26 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by preparative HPLC method to obtain the pure product as white solid (0.08 g, 10%). IR (cm⁻¹): 3442, 3230, 2937, 1695, 1485, 1394, 1297, 1186 and 1051. ¹HNMR (CDCl₃): δ 9.02-9.22 (s, 1H), 7.8-8.02 (m, 1H), 7.12 (d, 1H), 6.7-6.96 (m, 1H), 4.3-4.52 (m, 1H), 3.32-3.55 (m, 2H), 2.95-3.16 (m, 4H), 2.22-2.65 (m, 4H), 1.5-2.0 (m, 8H), 1.12-1.52 (m, 3H) and 0.8-1.02 (m, 3H). Mass: (M+1) 431 calculated for C₂₃H₃₁FN₄O₃.

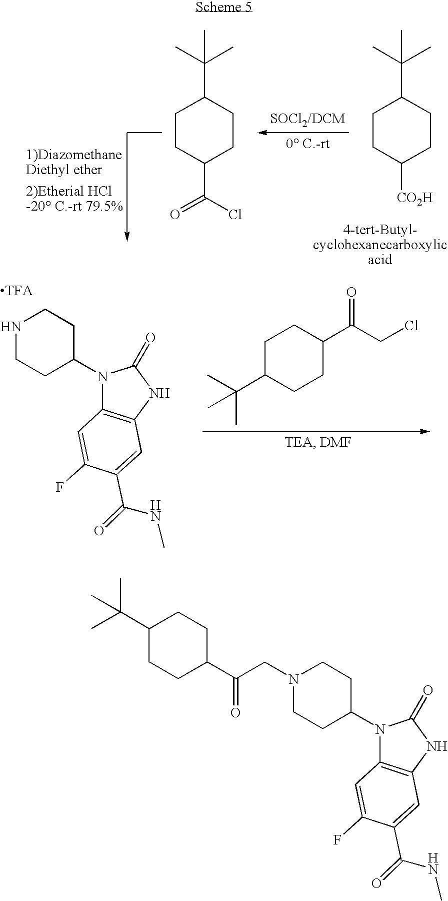

### Preparation of 4αrt-butylcyclohexanecarbonyl chloride

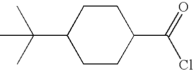

A solution of 4-tert-Butyl-cyclohexanecarboxylic acid (1.5 g, 8.13 mmol) (Chem. Abstr. Reg. No. 5451-55-8) in dry DCM (15 mL) was cooled to 0° C., SOCl₂ (1.76 mL, 24.4 mmol) was added. The reaction mixture was allowed to rt, maintained for 3 h and then was concentrated to obtain the crude product (1.65 g). The crude was directly used in the next step without any purification.

### Preparation of 1-(4-tert-butylcyclohexyl)-2-chloroethanone

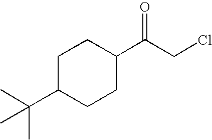

A solution of 4-tert-butylcyclohexanecarbonyl chloride (1.65 g) in diethyl ether (15 mL) was cooled to −20° C., a solution of diazomethane (2.45 g) in diethyl ether (100 mL) was added. The reaction mixture was allowed to 0° C. and maintained for 2 h. The resulted reaction mixture was cooled −20° C., ethereal HCl (15 mL) was added, allowed to rt, maintained for 2 h and concentrated to obtain the crude product (1.4 g, 79.5%). ¹HNMR (CDCl₃): δ 4.1-4.25 (m, 2H), 2.646-2.65 (m, 2H), 2.1-2.3 (m, 2H), 1.8-2.02 (m, 4H), 1.2-1.3 (m, 2H), 0.85 (s, 9H).

### EXAMPLE NO. 14

### 1-{1-[2-(4-tert-butylcyclohexyl)-2-oxoethyl]piperidin-4-yl}-6-fluoro-N-methyl-2-oxo-2,3-dihydro-1H-benzimidazole-5-carboxamide

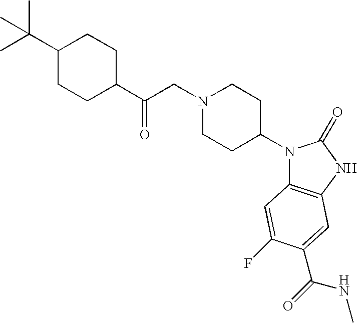

To a solution of the intermediate 4-{6-fluoro-5-[(methylamino)carbonyl]-2-oxo-2,3-dihydro-1H-benzimidazol-1-yl}piperidinium trifluoroacetate (0.35 g, 1.198 mmol) in dry DMF (5 mL), TEA (1.66 mL, 11.98 mmol) was added followed by the addition of 1-(4-tert-Butyl-cyclohexyl)-2-chloro-ethanone (0.31 g, 216 mmol) at room temperature and, and the reaction mixture was maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude product was washed with diethyl ether, filtered and dried to obtain the desired product as an off-white solid (0.07 g, 12.1%). IR (cm⁻¹): 3477, 3079, 2940, 1706, 1494, 1375, 1278 and 1147. ¹HNMR (CDCl₃): δ 8.75-9.02 (m, 1H), 7.85 (d, 1H), 7.1 (d, 1H), 6.72-6.9 (m, 1H), 4.3-4.46 (m, 1H), 3.26 (s, 2H), 2.98-3.28 (m, 5H), 2.2-2.5 (m, 5H), 1.78-2.0 (m, 6H), 1.3-1.5(m, 2H) and 0.85 (s, 9H). Mass: (M+1) 473 calculated for C₂₆H₃₇FN₄O₃.

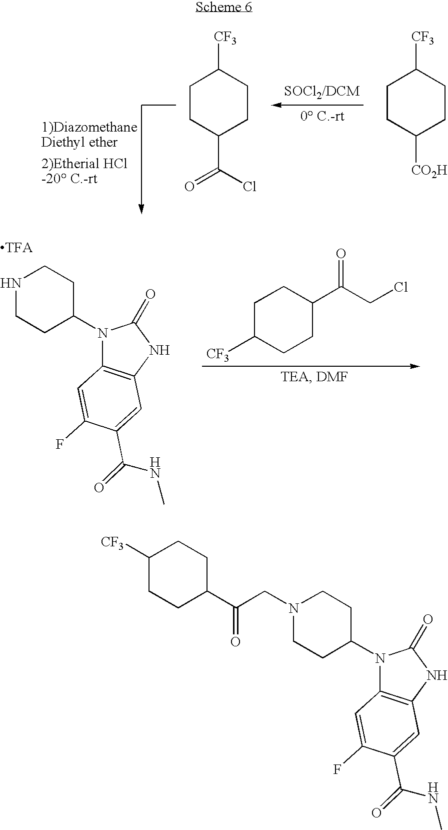

### Preparation of 4(trifluoromethyl)cyclohexanecarbonyl chloride

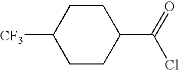

A solution of 4-Trifluoromethyl-cyclohexanecarboxylic acid d (1.5 g, 0.007 mmol) (Chem. Abstr. Reg. No. 95233-30-0) in dry DCM (10 mL) was cooled to 0° C., SOCl₂(0.655 mL, 0.009 mmol) was added. The reaction mixture was allowed to rt, maintained for 12 h and then was concentrated to obtain the crude product (1.64 g). The crude was directly used in the next step without any purification.

### Preparation of 2-chloro-1-[4(trifluoromethyl)cyclohexyl]ethanone (00-06)

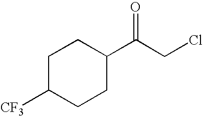

A solution of 4-Trifluoromethyl-cyclohexanecarbonyl chloride (1.64 g) in diethyl ether (5 mL) was cooled to −20° C., a solution of diazomethane (0.96 g) in diethyl ether (20 mL) was added. The reaction mixture was allowed to 0° C. and maintained for 2 h. The resulted reaction mixture was cooled −20° C., ethereal HCl (10 mL) was added, allowed to room temperature, maintained for 1 h and concentrated to obtain the crude product (0.8 g, 47%). ¹HNMR (CDCl₃): δ 4.12(s, 2H), 2.86-3.02 (m, 1H), 2.02-2.25 (m, 3H), 1.7-1.9 (m, 2H) and 1.52-1.72 (m, 4H).

### EXAMPLE NO. 15

### 6-fluoro-N-methyl-2-oxo-1-(1-{2-oxo-2-[4(trifluoromethyl)cyclohexyl]ethyl}piperidin-4-yl)-2,3-dihydro-1H-benzimidazole-5-carboxamide

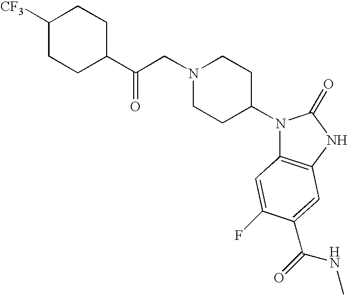

To a solution of product 4-{6-fluoro-5-[(methylamino)carbonyl]-2-oxo-2,3-dihydro-1H-benzimidazol-1-yl}piperidinium trifluoroacetate (0.4 g, 1.3698 mmol) in dry DMF (5 mL), TEA (1.9 mL, 13.69 mmol) was added followed by the addition of 2-Chloro-1-(4-trifluoromethyl-cyclohexyl)-ethanone (B) (0.37 g, 1.6437 mmol) at rt and maintained for 2 h. The reaction mass was diluted with water (10 mL) and extracted with EtOAc (3×20 mL). The organic layer was washed with water (3×50 mL), brine solution (50 mL), dried over anhydrous sodium sulfate and concentrated to obtain the crude product. The crude was purified by silica gel column using 3% MeOH in CHCl₃ as eluting solvent to obtain the product as an off-white solid (150 mg, 24.8%). IR (cm⁻¹): 3453, 3060, 2940, 1710, 1641, 1494, 1378, 1274, 1147 and 1089. ¹HNMR (CDCl₃): δ 8.98-9.32 (m, 1H), 7.86 (d, 1H), 7.06 (d, 1H), 6.7-7.9 (m, 1H), 4.22-4.5 (m, 1H), 3.26-3.45 (m, 1H), 2.96-3.28 (m, 4H), 1.9-2.56 (m, 4H), 1.52-1.9 (m, 5H), 1.2-1.9 (m, 4H) and 1.2-1.5 (m, 1H). Mass: (M+1) 485 calculated for C₂₃H₂₈F₄N₄O₃.

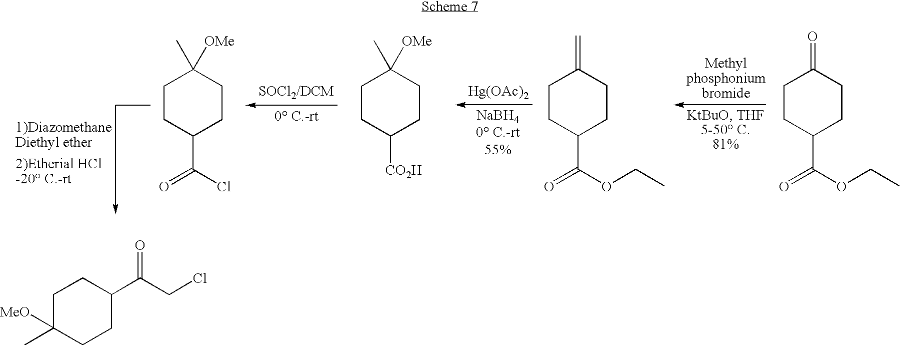

### Preparation of 4-Methylenecyclohexanecarboxylic acid ethyl ester

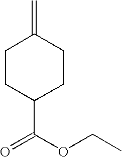

A suspension of methyl phosphonium bromide (3.15 g, 8.82 mmol) in dry THF (20 mL) was cooled to 0° C., a solution of KtBuO (1.185 g, 10.58 mmol) in dry THF (15 mL) was added. The reaction mixture was allowed to rt and maintained for 1 h. The resulting mixture was cooled to 5-10° C., 4-Oxo-cyclohexanecarboxylic acid ethyl ester (1.0 g, 5.88 mmol) (Chem. Abstr. Reg. No. 17159-79-4) was added over a period of 5 min, then was warmed to rt, maintained for 2 h, then was heated to 50° C. and maintained over night additionally. The resulted reaction mass was diluted with water (200 mL) and extracted with EtOAc (200 mL). The organic layer was washed with water (50 mL), brine solution (100 mL), dried over anhydrous sodium sulfate and concentrated to obtain the crude product. The crude was purified by column chromatography over silica gel column using 3-4% EtOAc in pet ether as eluting solvent to obtain the pure product (1) as liquid (800 mg, 81%). ¹HNMR (CDCl₃): δ 4.65(s, 2H), 4.12 (q, 2H), 2.3-2.5 (m, 3H), 1.9-2.16 (m, 4H), 1.5-1.7 (m, 2H) and 1.25(t, 3H). Mass: (M+1) 169 calculated for C₁₀H₁₆O₂.

### Preparation of 4-Methoxy-4-methylcyclohexanecarboxylic acid

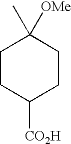

A suspension of Hg(OAc)₂ (2.2 g, 6.96 mmol) in dry methanol (15 mL) was cooled to 0° C. under stirring, 4-Methylenecyclohexanecarboxylic acid ethyl ester (0.9 g, 5.36 mmol) was added and maintained for 15 min. To the resulted reaction mixture, 3M NaOH (12 mL) was added followed by the addition of a mixture of NaBH₄ solution in 3M NaOH solution (12 mL) and maintained for 10 min. The reaction mixture was allowed to rt and maintained for 1 h additionally. The reaction mass was filtered through celite bed, the filtrate was cooled, acidified and then was extracted with DCM (100 mL). The organic layer was washed with water (2×50 mL), brine solution (50 mL), dried over anhydrous sodium sulfate and concentrated to obtain the crude product. The crude was purified by column chromatography over silica gel column using 10% EtOAc in pet ether as eluting solvent to obtain the pure product (2) as liquid (500 mg, 55%).

¹HNMR (CDCl₃): δ 3.22(s, 1H), 3.15 (s, 3H), 2.18-2.36 (m, 1H), 1.9 (d, 2H), 1.5-1.82 (m, 5H), 1.2-1.32 (m, 2H) and 1.1(s, 3H). Mass: (M−1) 171 calculated for C₉H₁₆O₃.

### Preparation of 4-Methoxy-4 methylcyclohexanecarbonyl chloride

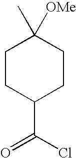

A solution of product 4-Methoxy-4-methylcyclohexanecarboxylic acid (0.6 g, 3.48 mmol) in dry DCM (5 mL) was cooled to 0° C., SOCl₂ (0.379 mL, 5.2 mmol) was added. The reaction mixture was allowed to rt, maintained over night and then was concentrated to obtain the crude product (3) (0.67 g). The crude was directly used in the next step without any purification.

### Preparation of 2-Chloro-1-(4-methoxy-4-methyl-cyclohexyl)-ethanone

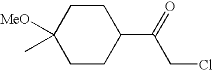

A solution of 4-Methoxy-4-methyl-cyclohexanecarbonyl chloride (0.67 g) in diethyl ether (5 mL) was cooled to −40° C., a solution of diazomethane (0.443 g) in diethyl ether was added. The reaction mixture was allowed to 0° C. and maintained for 2 h. The resulted reaction mixture was cooled −40° C., ethereal HCl (5 mL) was added and maintained for 30 min., allowed to rt, maintained for 1 h and concentrated to obtain the crude product. The crude was purified by column chromatography over silica gel column using 3-5% EtOAc-pet ether as eluting solvent to obtain the desired product as liquid (0.13 g, 18.3%). ¹HNMR (CDCl₃): δ 4.05(s, 2H), 3.2 (s, 3H), 2.65-2.8 (m, 1H), 1.82-1.96 (m, 2H), 1.5-1.75 (m, 6H) and 1.14(s, 3H).

### EXAMPLE NO. 16

### 6-fluoro-1-{1-[2-(4-methoxy-4-methylcyclohexyl)-2-oxoethyl]piperidin-4-yl}-N-methyl-2-oxo-2,3-dihydro-1H-benzimidazole-5-carboxamide

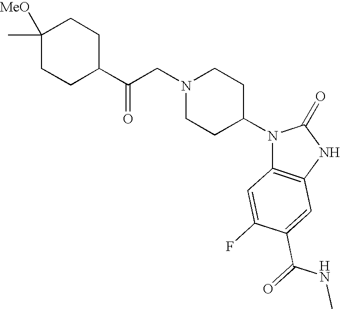

To a solution of the intermediate product 4-{6-fluoro-5-[(methylamino)carbonyl]-2-oxo-2,3-dihydro-1H-benzimidazol-1-yl}piperidinium trifluoroacetate (0.154 g, 0.52 mmol) in dry DMF (4 mL), TEA (0.73 mL, 5.2 mmol) was added followed by the addition of 2-Chloro-1-(4-methoxy-4-methyl-cyclohexyl)-ethanone (0.13 g, 0.63 mmol) at rt and maintained for 90 min. The reaction mass was diluted with water (20 mL) and extracted with EtOAc (2×20 mL). The organic layer was washed with water (3×20 mL), brine solution (20 mL), dried over anhydrous sodium sulfate and concentrated to obtain the crude product. The crude was purified by silica gel column using 3% methanol in CHCl₃ as eluting solvent to obtain the pure product as pale yellow solid (0.05 g, 20.6%). IR (cm⁻¹): 3465, 3160, 2940, 1687, 1402, 1178 and 1139.

¹HNMR (DMSO): δ 11.2(s, 1H), 9.82 (broad s, 1H), 8.02 (s, 1H), 7.46 (d, 1H), 7.26 (m, 1H), 4.3-4.7 (broad s, 3H), 3.56 (m, 2H), 3.02-3.3 (m, 5H), 2.6-2.85 (m, 4H), 1.75-2.02 (s, 4H), 1.4-1.7 (m, 6H), 1.3 (s, 2H) and 1.1(s, 3H). Mass: (M+1) 461 calculated for C₂₄H₃₃FN₄O₄.

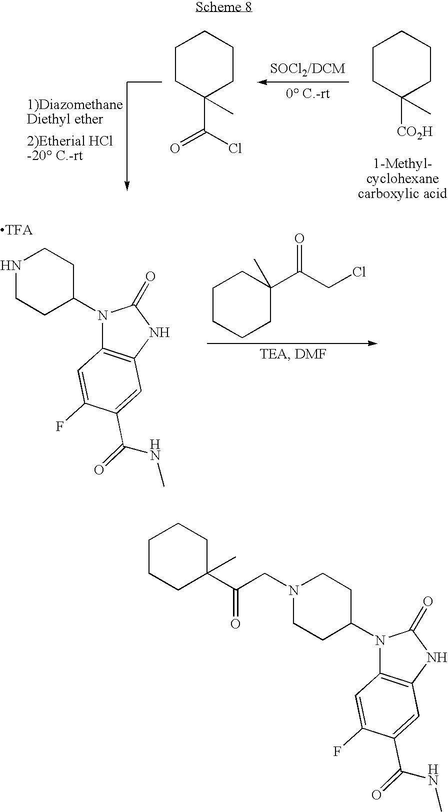

### Preparation of 1-Methyl-cyclohexanecarbonyl chloride

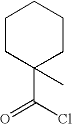

A solution of 1-Methyl-cyclohexanecarboxylic acid (1.5 g, 10.5 mmol) in dry DCM (15 mL) was cooled to 0° C., SOCl₂ (2.28 mL, 31.6 mmol) was added. The reaction mixture was allowed to rt, maintained for 3 h and then was concentrated to obtain the crude product (1.70 g). The crude was directly used in the next step without any purification.

### Preparation of 2-Chloro-1-(1-methyl-cyclohexyl)-ethanone

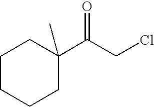

A solution of 1-Methyl-cyclohexanecarbonyl chloride (1.7 g) in diethyl ether (5 mL) was cooled to −20° C., a solution of diazomethane (1.33 g) in diethyl ether was added. The reaction mixture was allowed to 0° C. and maintained for 2 h. The resulted reaction mixture was cooled −20° C., ethereal HCl (8 mL) was added, allowed to rt, maintained for 2 h and concentrated to obtain the crude product (1.2 g, 65.2%).

¹HNMR (CDCl₃): δ 4.35(s, 2H), 1.9-2.02 (m, 2H), 1.22-1.65 (m, 8H) and 1.15(s, 3H).

### EXAMPLE NO. 17

### 6-fluoro-N-methyl-1-{1-[2-(1-methylcyclohexyl)-2-oxoethyl]piperidin-4-yl}-2-oxo-2,3-dihydro-1H-benzimidazole-5-carboxamide

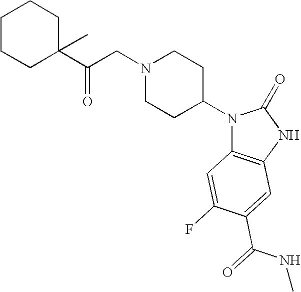

To a solution of 4-{6-fluoro-5-[(methylamino)carbonyl]-2-oxo-2,3-dihydro-1H-benzimidazol-1-yl}piperidinium trifluoroacetate (0.2 g, 0.68 mmol) in dry DMF (4 mL), TEA (0.951 mL, 6.8 mmol) was added followed by the addition of 2-Chloro-1-(1-methyl-cyclohexyl)-ethanone (0.143 g, 0.82 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by silica gel column using 2.4% MeOH in CHCl₃ as eluting solvent to obtain the pure product as pale grey solid (0.05 g, 17%). IR (cm⁻¹): 3469, 3326, 2929, 1714, 1633, 1486, 1282 and 1155. ¹HNMR (CDCl₃): δ 8.8(s, 1H), 7.84 (d, 1H), 7.14 (d, 1H), 6.84 (m, 1H), 4.42 (m, 1H), 3.5 (s, 2H), 3.0-3.22 (m, 5H), 1.78-2.04 (m, 3H), 1.22-1.75 (m, 12H), 1.12 (s, 3H) and 0.8-0.96 (m, 1H). Mass: (M+1) 431 calculated for C₂₃H₃₁FN₄O₃.

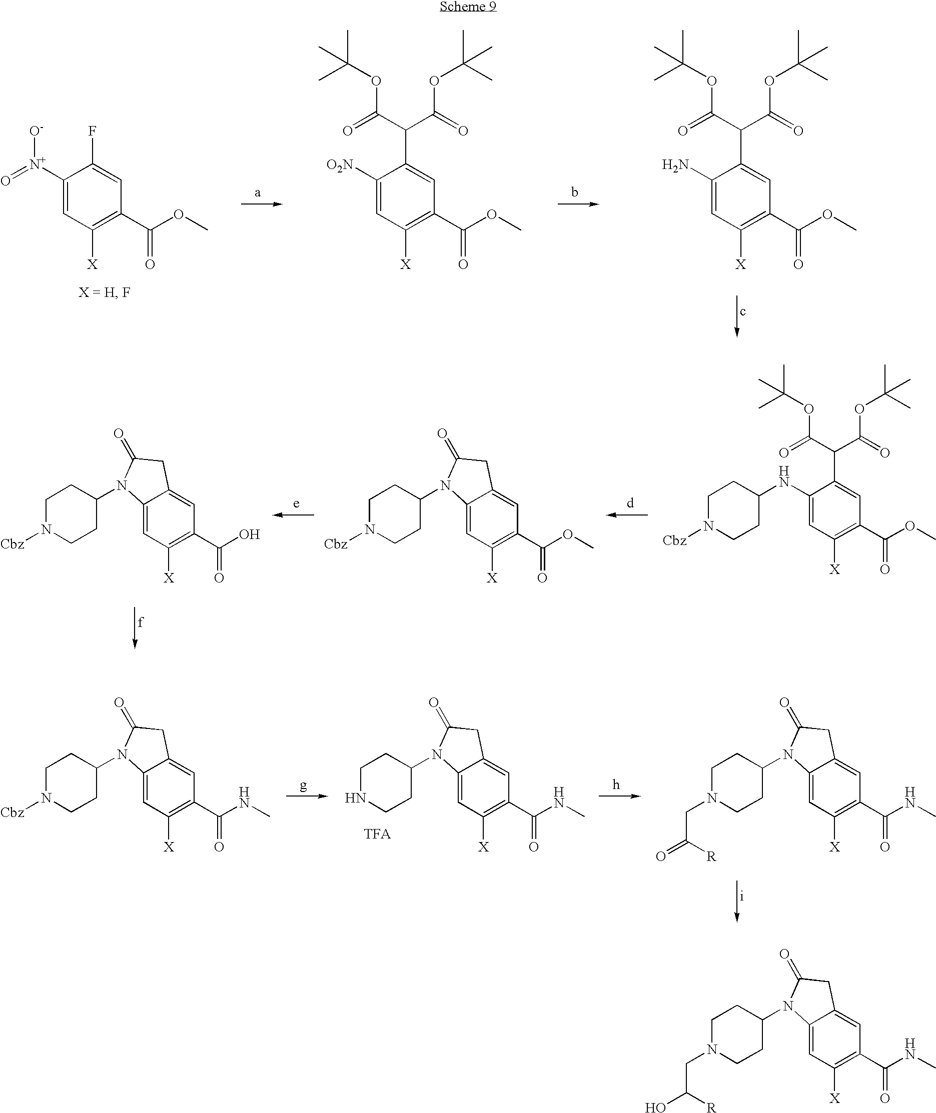

Reaction conditions: a) Di-t-butyl-malonate, NaH, DMF, 0° C., b) Raney Ni/MeOH c) N-Cbz-4-piperidone, DCE, Na(OAc)₃BH d) toluene sulfonic acid, toluene reflux e) NaOH/MeOH, 80° C., f) NH₂CH₃, EDC, HOBt, DMF g) 10% Pd/C, H₂, h) RC(O)CH₂Cl, DIEA, DMF rt, i) NaBH₄, EtOH. CDl/DCM rt

### Preparation of methyl 3-fluoro-4-nitrobenzoate

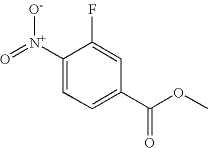

3-Fluoro-4-nitrobenzoic acid (8.15 g, 44.0 mmol) was dissolved in 100 mL anhydrous methanol under a nitrogen atmosphere, added 1 mL concentrated sulfuric acid, and subsequently heated at reflux for 5.5 hours. The reaction mixture was cooled to ambient temperature, concentrated in vacuo. Re-dissolved in diethyl ether (250 mL), washed with saturated sodium bicarbonate (3×100 mL), brine, dried with magnesium sulfate, filtered and concentrated in vacuo to yield title compound. (7.8 g, 89%): APCl⁻ 199.3; Anal. HPLC Retention time=16.6 minutes (>99% pure).

### Preparation of di-tert-butyl[5-(methoxycarbonyl)-2-nitrophenyl]malonate

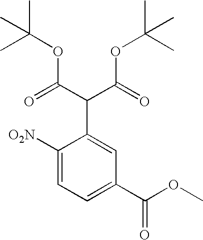

Di-t-butyl-malonate (8.8 g, 40.8 mmol) was dissolved in 80 mL anhydrous N,N-dimethylformamide under a nitrogen atmosphere, cooled to 0° C., then added sodium hydride (60% dispersion in oil, 1.9 g, 47 mmol) in portions. Evolution of gas noted. The reaction mixture was stirred at 0° C. for 20 minutes and methyl 3-fluoro-4-nitrobenzoate (5.8 g, 29 mmol) was added in portions. Cooling bath was removed after 20 minutes and the reaction mixture was warmed to ambient temperature overnight. After concentrated under reduced pressure the reaction mixture was diluted with ethyl acetate and was partitioned between ethyl acetate and water, washing organics with more water, brine, dried with magnesium sulfate, filtered and concentrated in vacuo. The desired product was purified by column chromatography eluting with a gradient of 0-50% ethyl acetate in hexanes. The fractions were combined and concentrated containing product to a yellow solid. (9.15 g, 79%): APCl⁻ 395.33; Anal. HPLC Retention time=21.8 minutes.

### Preparation of di-tert-butyl[2-amino-5-(methoxycarbonyl)phenyl]malonate

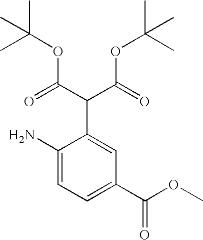

di-tert-butyl[5-(methoxycarbonyl)-2-nitrophenyl]malonate (9.1 g, 23 mmol) was dissolved in methanol. To this solution, was added Raney Nickel and the reaction mixture was agitated. The reaction mixture was concentrated, after the solution was filtered through a pad of celite, in vacuo to yield title compound. (8.2 g, 88%): APCl⁺ 366.15; Anal. HPLC Retention time=19.7 minutes (88% pure).

### Preparation of di-tert-butyl[2-({1-[(benzyloxy)carbonyl]piperidin-4-yl}amino)-5-(methoxycarbonyl)phenyl]malonate

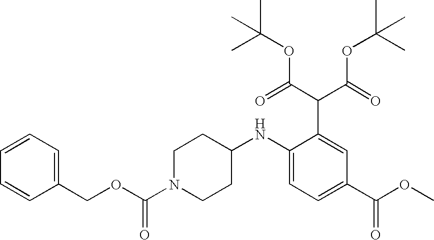

di-tert-butyl[2-amino-5-(methoxycarbonyl)phenyl]malonate (4.3 9.12 mmol) and N-benzyloxycarbonyl-4-piperidone (5.5 g, 24 mmol) were combined in 40 mL 1,2-dichloroethane (DCE) under a nitrogen atmosphere, added acetic acid (3.4 mL, 58.8 mmol), followed by 0.5 equivalents of sodium triacetoxyborohydride (1.2 g, 5.9 mmol). The reaction mixture was stirred at ambient temperature for 4 hours. Additional sodium triacetoxyborohydride (3.6 g, 17.7 mmol) was added by small portions over 3 days until the reaction was complete. The reaction was quenched with the careful addition of 100 mL sat. sodium bicarbonate. Evolution of gas was noted. The reaction mixture was extracted with dichloromethane and this was washed with brine, dried with magnesium sulfate and filtered. The filtrate was concentrated in vacuo. The desired product was purified by column chromatography eluting with a gradient of 0-100% ethyl acetate in hexanes. The desired fractions were combined and concentrated to yield title compound. (5.6 g, 82%) APCl⁺ 583.29; Anal. HPLC Retention time=23.3 minutes.

### Preparation of methyl 1-{1-[(benzyloxy)carbonyl]piperidin-4-yl}-2-oxoindoline-5-carboxylate

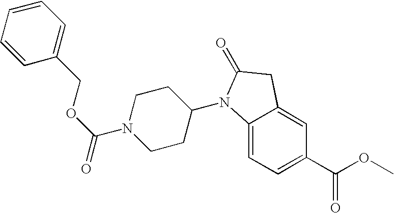

di-tert-butyl[2-({1-[(benzyloxy)carbonyl]piperidin-4-yl}amino)-5(methoxycarbonyl)phenyl]malonate (5.6 g, 9.6 mmol) was dissolved in 50 mL anhydrous toluene, added p-toluenesulfonic acid monohydrate (0.37 g, 1.9 mmol), then refluxed for 2 hours. The solution was cooled and concentrated in vacuo. The resultant crude material was purified by column chromatography eluting with a gradient of 20-100% ethyl acetate in hexanes. The pure fractions were combined and concentrated to yield title compound. (3.18 g, 81%) APCl⁺ 409.18.

### Preparation of 1-{1-[(benzyloxy)carbonyl]piperidin-4-yl}-2-oxoindoline-5-carboxylic acid

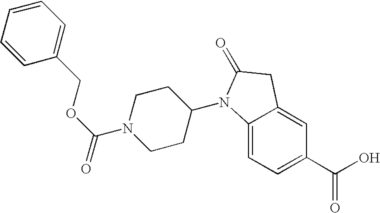

methyl 1-{1-[(benzyloxy)carbonyl]piperidin-4-yl}-2-oxoindoline-5-carboxylate (1.5 g, 3.7 mmol) was suspended in 50 mL methanol, heated to 80° C. which caused solids to dissolve, added 2M sodium hydroxide and stirred at 80° C. for 2 hours. The solution was cooled to ambient temperature, acidified with 1M aq. hydrogen chloride, extracted three times with dichloromethane, dried combined organics with magnesium sulfate, filtered and concentrated to yield title compound. (1.4 g, 97%) APCl⁺ 395.15; Anal. HPLC Retention time=18.7 minutes.

### Preparation of benzyl 4-{5-[(methylamino)carbonyl]-2-oxo-2,3-dihydro-1H-indol-1-yl}piperidine-1-carboxylate

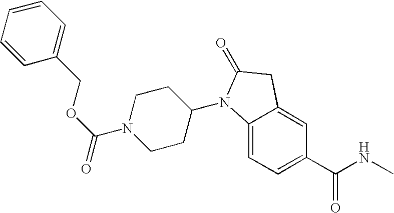

1-{1-[(benzyloxy)carbonyl]piperidin-4-yl}-2-oxoindoline-5-carboxylic acid (1.4 g, 3.5 mmol) was dissolved in 20 mL anhydrous dichloromethane under a nitrogen atmosphere, added 1-hydroxybenzotriazole hydrate (0.82 g, 5.3 mmol) and N-(3-dimethylaminopropyl)-N′-ethylcarbodiimide hydrochloride (1.0 g, 5.3 mmol), stirred for 10 minutes then added 2M methylamine in tetrahydrofuran solution (7.1 mL, 14 mmol). The reaction mixture was kept at ambient temperature for additional 3 days. Diluted with 100 mL dichloromethane, washed with sat. sodium bicarbonate (3×50 mL), 1M aq. hydrogen chloride (3×50 mL), brine, dried with magnesium sulfate, filtered and concentrated in vacuo. The desired product was purified by column chromatography eluting with a gradient of 0-25% methanol in ethyl acetate. The desired fractions were combined and concentrated to yield title compound. (0.81 g, 56%) APCl⁺ 408.18; Anal. HPLC Retention time=15.9 minutes.

### Preparation of N-methyl-2-oxo-1-piperidin-4-ylindoline-5-carboxamide

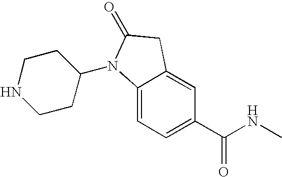

benzyl 4-{5-[(methylamino)carbonyl]-2-oxo-2,3-dihydro-1H-indol-1-yl}piperidine-1-carboxylate (0.81 g, 1.99 mmol) was dissolved in methanol (25 mL) and tetrahydrofuran (25 mL), added 10% palladium on carbon (0.1 g) and shaken for 10 hours. The filtered solution was concentrated to yield title compound. (0.36 g, 66%) APCl⁺ 274.18; Anal. HPLC Retention time=10.1 minutes.

### Preparation of (3aR,6aS)-2-(chloroacetyl)octahydrocyclopenta[c]pyrrole

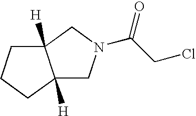

(3aR,6aS)-octahydrocyclopenta[c]pyrrole (10 g, 68 mmol) was dissolved in 70 mL ethyl acetate, added 2M aq. sodium carbonate followed by the addition of chloroacetyl chloride (5.4 mL, 68 mmol) in small portions. The reaction mixture was stirred at ambient temperature for 2 hours. Added 100 mL water, separated layers, extracted aqueous once with 100 mL ethyl acetate, washed combined organics with brine, dried with magnesium sulfate, filtered and concentrated to yield title compound. (10.2 g, 80%) APCl⁺ 188.12, 190.10 (Cl pattern).

### Preparation of 4,4-dimethylpiperidine

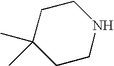

A 500 mL round bottomed flask was charged with solid lithium aluminum hydride (2.5 g, 64 mmol) under an argon atmosphere, 40 mL of anhydrous tetrahydrofuran was added slowly. The suspension was cooled to 0° C., then added a solution of 3,3-dimethylglutarimide (3.0 g, 21 mmol) in 20 mL anhydrous tetrahydrofuran slowly. Evolution of gas was observed. After complete addition the ice bath was removed and allowed to warm to ambient temperature. The flask was equipped with a condenser and placed in an oil bath and heated at reflux for 3 h. The solution was cooled to ambient temperature, placed flask in a bath of cool water for heat sink, then successive dropwise addition of 2.5 mL water, 2.5 mL of 15% aq. sodium hydroxide, and 7.5 mL of water resulted in a thick suspension. Added 150 mL dry Et₂O, stirred, then filtered off solids. Separated layers, dried organics with sodium sulfate and filtered. Cooled the organic solution in an ice bath and bubbled anhydrous hydrogen chloride (g) for −30 seconds. A thick white precipitate quickly forms. The solid was filtered off to yield title compound as hydrochloride salt. (2.68 g, 85%) APCl⁺; 114.1.

### Preparation of 2-chloro-1-(4,4-dimethylpiperidin-1-yl)-ethanone

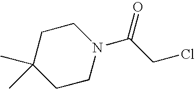

A 250 mL round bottomed flask was charged with 4,4-dimethyl-piperidine hydrochloride (6.2 g, 41 mmol), added 2M aq. sodium bicarbonate solution and 40 mL ethyl acetate. After the solids dissolved, chloroacetyl chloride (3.3 mL, 41 mmol) was added dropwise to the biphasic solution. The reaction mixture was stirred at ambient temperature for 90 minutes, and was diluted with 100 mL ethyl acetate and 100 mL water, separated, extracted aqueous once with 100 mL ethyl acetate, washed combined extracts with brine, dried organic with magnesium sulfate, filtered and concentrated in vacuo. Redissolved in 40 mL hexanes and concentrated yield title compound. (6.2 g, 79%) APCl⁺ 190.1, 192.1 (Cl pattern).

### EXAMPLE NO. 18

### 1-{1-[2-(4-methoxy-4-methylpiperidin-1-yl)-2-oxoethyl]piperidin-4-yl}-N-methyl-2-oxoindoline-5-carboxamide

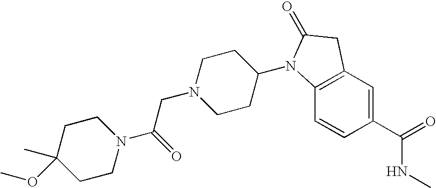

The amine (6.19 g, 22.6 mmol) was suspended in dry DMF (115 mL) and cooled to 0° C. under an atmosphere of nitrogren. Triethylamine (5.26 mL, 37.7 mmol) was added, and the mixture stirred for 15 min. The chloride (3.88 g, 18.9 mmol) was dissolved in 25 mL of dry DMF and added dropwise to the amine mixture over 20 min. via an addition funnel. The mixture was stirred vigorously at RT for 15 h and then concentrated under reduced pressure with heat. The resulting oil was dissolved in dichloromethane and washed with 5% aqueous NaHCO₃. The aqueous layer was extracted twice with dichloromethane. All the organic extracts were combined and washed with water three times followed by brine and then dried over Na₂SO₄. The mixture was filtered and then concentrated under reduced pressure to give an oil which was diluted with dichloromethane and purified by silica gel chromatography (5/95-50/50 MeOH/EtOAc). The product (5.2 g, 65%) was isolated as a yellow solid. Use the product as is in the next reaction. NMR (400 MHz, DMSO-d₆): δ 8.26 (m, 1H), 7.74 (dd, 1H, J=2, 8 Hz), 7.70 (d, 1H, J=1.3 Hz), 7.11 (d, 1H, J=2 Hz), 4.04-4.00 (m, 1H), 3.86-3.83 (m, 1H), 3.66 (m, 1H), 3.53 (s, 2H), 3.24-3.16 (m, 2H), 3.08 (s, 3H), 2.90-2.85 (m, 3H), 2.70 (d, 3H, J=4 Hz), 2.31-2.27 (m, 2H), 2.12-2.10 (m, 2H), 1.8-1.4 (m, 5H), 1.08 (s, 3H). MS: APCI (AP+): 443.3 (M+H)⁺.

### EXAMPLE NO. 19

### 1-[2-(4-methoxy-4-methylpiperidin-1-yl)-2-oxoethyl]-4-{5-[(methylamino)carbonyl]-2-oxo-2,3-dihydro-1H-indol-1-yl}piperidinium chloride

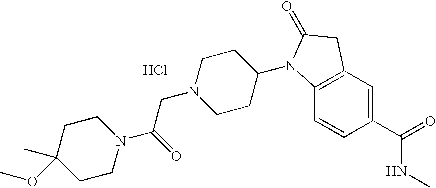

The starting material was dissolved in dichloromethane (300 mL) and methanol (25 mL). And the solution cooled in an ice bath to 0° C. HCl gas was bubbled slowly into the reaction for 10 min. The ice bath was removed, and the reaction stirred at RT for 30 min. followed by concentration under reduced pressure to yield the product as yellow foam. This product was dissolved in 200 mL of methanol, and ether was added slowly with stirring until solids began to precipitate from solution. The mixture was cooled in an ice bath for 10 min, and more ether was added slowly with stirring. The solids were filtered and washed with ether. The collected hygroscopic solids were put into a flask and dried under vacuum at RT and then further dried in a vacuum oven at 60° C. for 48 h to yield the product (4.5 g, 80%) as a pale yellow solid. CHN calc'd for C₂₄H₃₄N₄O₄+1.05 HCl+1.3H₂O+0.1 C₄H₁₀₀(diethyl ether): % C, 57.28; % H, 7.61; % N, 10.95; % Cl 7.28; % H₂O 4.58. Found: % C, 57.35; % H, 7.52; % N, 10.92; % Cl 7.49; % H₂O 4.51. MS: APCl (AP+): 443.3 (M+H)⁺.

### EXAMPLE NO. 20

### 1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium hydrogen sulfate

1) 4-(4-methoxycarbonyl-phenylamino)-piperidine-1-carboxylic acid benzyl ester

Methyl 4-aminobenzoate (10.11 g, 66.88 mmol), benzyl 4-oxo-1-piperidinecarboxylate (18.58 g, 79.61 mmol) and acetic acid (4.0 mL, 69.88 mmol) were added to dichloromethane (100 mL). Sodium triacetoxyborohydride (21.05 g, 99.32 mmol) was added portionwise over a period of two hours. The mixture was stirred at room temperature for four hours and then slowly added to 5% aq. Sodium hydroxide solution. The mixture was stirred for 1 hour. The organic layer was separated, washed with water and concentrated to dryness. Methyl t-butyl ether (100 mL), followed by heptane (100 mL), was added. The slurry was stirred and filtered. The cake was washed with 1:1 heptane/Methyl t-butyl ether and dried under vacuum to give 4-(4-Methoxycarbonyl-phenylamino)-piperidine-1-carboxylic acid benzyl ester (19.71 g) as a white powder in 80% yield. LC-MS APCl (m/z) 369 (M+H)⁺; ¹H NMR (400 MHz, CDCl₃): δ 7.85 (d, 2H), 7.35 (m, 5H), 6.54 (d, 2H), 5.13 (s, 2H), 4.15 (m, 2H), 3.84 (s, 3H), 3.51 (m, 1H), 3.01 (m, 2H), 2.06 (m, 2H), 1.38 (m, 2H).

### 2) 4-[(2-chloro-acetyl)-(4-methoxycarbonyl-phenyl)-amino]-piperidine-1-carboxylic acid benzyl ester

4-(4-Methoxycarbonyl-phenylamino)-piperidine-1-carboxylic acid benzyl ester (101.00 g, 274 mmol), pyridine (33.3 mL, 412 mmol) were added to ethyl acetate (1 L). Chloroacetyl chloride (33.0 mL, 414 mmol) was added slowly. The mixture was stirred at room temperature for 1 hour. Water was added and the mixture was stirred for 10 min. The organic layer was separated, washed with 10% aq. sodium chloride and concentrated to dryness. Methyl t-butyl ether (600 mL) was added. The slurry was stirred at room temperature for 1 hour and filtered. The cake was washed with Methyl t-butyl ether and dried under vacuum to give 4-[(2-chloro-acetyl)-(4-methoxycarbonyl-phenyl)-amino]-piperidine-1-carboxylic acid benzyl ester (109.71 g) as a white powder in 90% yield. LC-MS APCl (m/z) 446 (M+H)⁺; ¹H NMR (400 MHz, CDCl₃): δ 8.12 (d, 2H), 7.28 (m, 5H), 7.21 (d, 2H), 5.02 (brs, 2H), 4.77 (m, 1H), 4.21 (m, 2H), 3.95 (s, 3H), 3.67 (m, 2H), 2.86 (m, 2H), 1.82 (m, 2H), 1.25 (m, 2H).

### 3) Methyl 1-{1-[(benzyloxy)carbonyl]piperidin-4-yl}-2-oxoindoline-5-carboxylate

4-[(2-chloro-acetyl)-(4-methoxycarbonyl-phenyl)-amino]-piperidine-1-carboxylic acid benzyl ester (90 g, 202.3 mmol), Pd(OAc)₂ (2.27 g, 10.1 mmol) and 2-(di-t-butylphosphino)biphenyl (6.04 g, 20.23 mmol) were added to 2-Methyl tetrahydrofuran (900 mL) and isopropanol (180 mL). The system was purged with nitrogen. Triethylamine (42.3 mL 303.4 mmol) was added. The mixture was heated to 80° C. and stirred at this temperature for 2 hours. The mixture was filtered in hot through Celite with methyl t-butyl ether washing. The combined filtrates were cooled to 15° C. and stirred for 1 hour. The slurry was filtered. The cake was washed with Methyl t-butyl ether and dried under vacuum to give methyl 1-{1-[(benzyloxy)carbonyl]piperidin-4-yl}-2-oxoindoline-5-carboxylate (66.1 g) as an off-white powder in 80% yield. LC-MS APCl (m/z) 409 (M+H)⁺; ¹H NMR (400 MHz, CDCl₃): δ 7.95 (dd, 1H), 7.90 (d, 1H), 7.38 (m, 5H), 6.96 (d, 1H), 5.16 (s, 2H), 4.42 (m, 3H), 3.89 (s, 3H), 3.55 (s, 2H), 2.90 (m, 2H), 2.33 (m, 2H), 1.72 (m, 2H).

### 4) 1-{1-[(benzyloxy)carbonyl]piperidin-4-yl}-2-oxoindoline-5-carboxylic acid

Methyl 1-{1-[(benzyloxy)carbonyl]piperidin-4-yl}-2-oxoindoline-5-carboxylate (27.0 g, 66.1 mmol) was added to acetonitrile (270 mL) and water (270 mL). The mixture was heated to 60° C. 1N aq. sodium hydroxide (67 mL, 67 mmol) was added. The mixture was stirred at 72° C. for 2 hours. The mixture was cooled to 50° C. A solution of acetic acid (14 mL, 244.6 mmol) in water (100 mL) was added. The slurry was stirred at room temperature for 1 hour and filtered. The cake was washed with water and dried under vacuum to give 1-(1-[(benzyloxy)carbonyl]piperidin-4-yl)-2-oxoindoline-5-carboxylic acid (23.2 g) as an off-white powder in 89% yield. LC-MS APCl (m/z) 395 (M+H)⁺; ¹H NMR (400 MHz, CDCl₃): δ 8.07 (dd, 1H), 8.00 (d, 1H), 7.42 (m, 5H), 7.04 (d, 1H), 5.21 (s, 2H), 4.47 (m, 3H), 3.61 (s, 2H), 2.95 (m, 2H), 2.35 (m, 2H), 1.72 (m, 2H).

### 5) Benzyl 4-{5-[(methylamino)carbonyl]-2-oxo-2,3-dihydro-1H-indol-1-yl}piperidine-1-carboxylate

1-{1-[(benzyloxy)carbonyl]piperidin-4-yl}-2-oxoindoline-5-carboxylic acid (30.0 g, 76.1 mmol) and CDl (19.0 g, 117 mmol) were added to acetonitrile (300 mL). The mixture was stirred at room temperature for 1 hour. 2M methylamine solution in tetrahydrofuran (90 mL, 180 mmol) was added. The mixture was stirred at room temperature for 1 hour. 1N aq. hydrochloric acid (300 mL) was added. The mixture was vacuum distilled to remove acetonitrile. The aqueous layer was extracted with dichloromethane. The organic layer was washed with 0.3N aq. sodium hydroxide and concentrated to dryness. Ethyl acetate (120 mL), followed by heptane (120 mL) was added. The slurry was stirred at room temperature for 2 hours and filtered. The cake was washed with 1:1 ethyl acetate/h heptane and dried under vacuum to give benzyl 4-{5-[(methylamino)carbonyl]-2-oxo-2,3-dihydro-1H-indol-1-yl}piperidine-1-carboxylate (26.0 g) as an off-white powder in 84% yield. LC-MS APCl (m/z) 408 (M+H)⁺; ¹H NMR (400 MHz, CD₃OD): [symbol] 8.33 (brs, 1H), 7.74 (d, 1H), 7.70 (s, 1H), 7.37 (m, 5H), 7.11 (d, 1H), 5.14 (s, 2H), 4.31 (m, 3H), 3.56 (s, 2H), 2.95 (m, 2H), 2.88 (s, 3H), 2.35 (m, 2H), 1.71 (m, 2H).

### 6) (3aR,6aS)-2-(chloroacetyl)octahydrocyclopenta[c]pyrrole

3-azabicyclo[3,3,0]octane HCl (10.0 g, 67.7 mmol) was added to dichloromethane (120 mL). The mixture was cooled to 5° C. 2M aq. potassium carbonate (69.8 mL, 139.6 mmol) was added slowly to keep the mixture below 10° C. The mixture was cooled to 5° C. and a solution of chloroacetyl chloride (8.4 g) in dichloromethane (20 mL) was slowly added to keep the mixture below 10° C. The mixture was stirred for 2 hours at 10° C. The organic layer was isolated, washed with 2M aq. potassium carbonate and brine, and concentrated to dryness to give (3aR,6aS)-2-(chloroacetyl)octahydrocyclopenta[c]pyrrole (12.07 g) as a light brown oil in 95% yield. This oil was directly used in next step without further purification. LC-MS APCl (m/z) 189 (M+H)⁺; ¹H NMR (400 MHz, CD₃OD): δ 4.14 (s, 2H), 3.73 (m, 1H), 3.61 (m, 1H), 3.35 (m, 1H), 3.28 (m, 1H), 2.77 (m, 1H), 2.67 (m, 1H), 1.87 (m, 2H), 1.75 (m, 1H), 1.63 (m, 1H), 1.47 (m, 2H).

### 7) N-methyl-2-oxo-1-piperidin-4-ylindoline-5-carboxamide

Benzyl 4-{5-[(methylamino)carbonyl]-2-oxo-2,3-dihydro-1H-indol-1-yl}piperidine-1-carboxylate (20.0 g, 49.1 mmol), 10% Pd/c (50% wet, 5.0 g) and methanol (300 mL) were added to 500 mL Parr Bottle. The mixture was shaken under 40 psi of hydrogen for 3 hours. The mixture was filtered through Celite pad with methanol washing. The combined filtrates were concentrated to dryness to give N-methyl-2-oxo-1-piperidin-4-ylindoline-5-carboxamide (13.42 g), as colorless oil in 100% yield. This oil was directly used for next step without further purification. LC-MS APCl (m/z) 274 (M+H)⁺; ¹H NMR (400 MHz, CD₃OD): δ 7.75 (d, 1H), 7.71 (s, 1H), 7.30 (d, 1H), 4.34 (m, 1H), 3.28 (s, 2H), 3.15 (m, 2H), 2.88 (s, 3H), 2.70 (m, 2H), 2.39 (m, 2H), 1.67 (m, 2H).

### 8) 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide

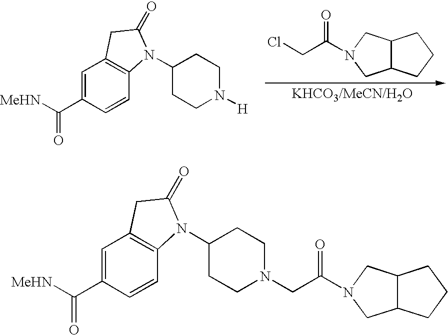

N-methyl-2-oxo-1-piperidin-4-ylindoline-5-carboxamide (13.42 g, 49.1 mmol) was dissolved to acetonitrile (300 mL). A solution of potassium bicarbonate (8.01 g, 80 mmol) in water (80 mL) was added. The mixture was heated to 60° C. A solution of (3aR,6aS)-2-(chloroacetyl)octahydrocyclopenta[c]pyrrole (10.00 g, 53.5 mmol) in acetonitrile (40 mL plus 20 mL rinse) was added. The mixture was then heated to 72° C. and stirred for 1.5 hours. The mixture was cooled to 50° C. Water (200 mL) was added and acetonitrile was distilled off under vacuum. 2-methyl tetrahydrofuran (120 mL was added. The slurry was stirred at 22° C. for 3 hours and filtered. The cake was washed with water and 2-methyl tetrahydrofuran and dried under vacuum to give 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide (13.5 g) as an off-white powder in 65% yield. LC-MS APCl (m/z) 425 (M+H)⁺; ¹H NMR (400 MHz, CD₃OD): δ 7.76 (dd, 1H), 7.72 (d, 1H), 7.36 (d, 1H), 4.27 (m, 1H), 3.71 (dd, 1H), 3.57 (m, 2H), 3.41 (dd, 1H), 3.28 (m, 2H), 3.22 (dd, 2H), 3.08 (m, 2H), 2.88 (s, 3H), 2.78 (m, 1H), 2.63 (m, 1H), 2.56 (m, 2H), 2.27 (m, 2H), 1.86 (m, 2H), 1.75 (m, 1H), 1.65 (m, 3H), 1.46 (m, 2H).

### 9) 1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium hydrogen sulfate

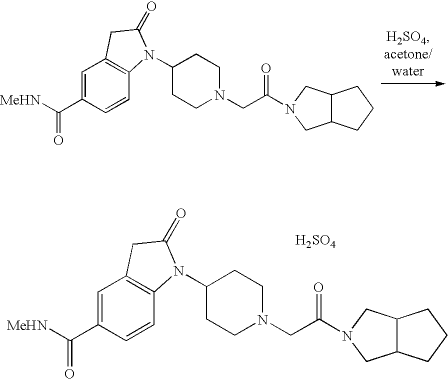

1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide (20.0 g, 47.1 mmol) was added to acetone (60 mL) and water (30 mL). The slurry was heated to 50° C. 3M aq. sulfuric acid (15.7 mL, 47.1 mmol) was added. The solution was stirred at 50° C. for 1 hour Acetone (340 mL) was added slowly. The slurry was stirred at 56° C. for 3 hours, 22° C. for 2 hours, and filtered. The cake was washed with acetone and dried under vacuum to give 1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium hydrogen sulfate (19.7 g) as a white powder in 80% yield. LC-MS APCl (m/z) 425 (M+H)⁺; ¹H NMR (400 MHz, D₂O): 7.47 (d, 1H), 7.41 (s, 1H), 7.02 (d, 1H), 4.27 (m, 1H), 3.96 (s, 2H), 3.57 (m, 2H), 3.40 (m, 3H), 3.07 (m, 4H), 2.67 (s, 3H), 2.60 (m, 3H), 2.48 (m, 2H), 1.86 (m, 2H), 1.63 (m, 2H), 1.49 (m, 1H), 1.41 (m, 1H), 1.23 (m, 2H).

### Method for Collecting Powder X-Ray Diffraction for the Above Compound.

### Powder X-Ray Diffraction Pattern

Powder x-ray diffraction pattern was collected for the above compound using a Bruker D5000 diffractometer (Madison Wis.) equipped with a copper radiation source, fixed slits (divergence 1.0 mm, antiscatter 1.0 mm, and receiving 0.6 mm) and a Solex solid-state detector. Data was collected in the theta-two theta goniometer configuration from a flat plate sample holder at the Copper wavelength Kα₁=1.54056 and Kα₂=1.54439 (relative intensity 0.5) from 3.0 to 40.0 degrees two-theta using a step size of 0.040 degrees and a step time of one second. X-ray tube voltage and amperage are preferably set at 40 kV and 40 mA respectively. Data were collected and analyzed using Bruker DIFFRAC Plus software. Samples were prepared by placing them in a quartz holder. (It is noted that a Bruker D5000 diffractometer is similar in operation to Siemans model D5000.) The results are summarized in Table 1 which provides the two-theta values and relative intensities for all of the reflections (lines) that have a relative intensity greater than or equal to 8% using a reflection width of 0.40 and a threshold of 1.0.

TABLE 1
Powder x-ray diffraction reflections for the above compound.

| | Relative |
|---|---|
| Angle | Intensity |
| 2-Theta ± 0.2° | % |
| 10.4 | 9.8 |
| 10.8 | 44.6 |
| 11.3 | 45.6 |
| 12.0 | 10 |
| 13.2 | 8.2 |
| 13.9 | 34.4 |
| 14.3 | 22 |
| 15.3 | 25.4 |
| 16.2 | 35.8 |
| 16.8 | 80.9 |
| 17.2 | 27 |
| 17.4 | 25.7 |
| 17.9 | 82.7 |
| 18.5 | 9.2 |
| 19.8 | 95.3 |
| 20.4 | 16.5 |
| 20.7 | 32.8 |
| 21.1 | 15.3 |
| 21.5 | 11.5 |
| 22.4 | 21.6 |
| 22.8 | 14.8 |
| 23.2 | 32.8 |
| 23.8 | 44.8 |
| 24.1 | 22.7 |
| 24.6 | 100 |
| 25.4 | 12.3 |
| 25.8 | 14.5 |
| 26.8 | 15.5 |
| 27.1 | 24.5 |
| 27.3 | 34.1 |
| 27.9 | 26 |
| 28.6 | 15.5 |
| 29.0 | 15.8 |
| 29.5 | 12 |
| 29.7 | 9.6 |
| 32.4 | 8.9 |
| 32.9 | 10.4 |
| 34.0 | 9.2 |
| 35.5 | 8.1 |
| 10.4 | 9.8 |

Alternatively the compound of Example 20 can be made by the following three steps followed by steps 6-9 as described above.

1) 4-(4-methylaminocarbonyl-phenylamino)-piperidine-1-carboxylic acid benzyl ester hydrogen chloride 4-amino-N-methylbenzamide (7.51 g, 50.0 mmol), benzyl 4-oxo-1-piperidinecarboxylate (14.0 g, 60.0 mmol) and acetic acid (2.86 mL, 50.0 mmol) were added to dichloroethane (90 mL). Sodium triacetoxyborohydride (15.9 g, 75 mmol) was added in three portions over a period of 6 hours. The mixture was stirred at room temperature for 18 hours and then slowly added to water (20 mL). 3M aq. sodium hydroxide (50 mL) was added to adjust pH to 13. The organic layer was isolated and the aqueous layer was extracted with dichloromethane. The combined organic layers were washed with sat. aq. sodium bicarbonate and brine, and dried over magnesium sulfate. The filtrates were concentrated to dryness. Methanol (90 mL) was added. The solution was heated to 70° C. 4.5M hydrogen chloride in isopropanol (12 mL, 54 mmol) was added. The slurry was stirred at 70° C. for 2 hours, 22° C. for 1 hour, and then filtered. The cake was washed with Methyl t-butyl ether and dried under vacuum to give 4-(4-methylaminocarbonyl-phenylamino)-piperidine-1-carboxylic acid benzyl ester hydrogen chloride (17.4 g) as a white powder in 86% yield. LC-MS APCl (m/z) 368 (M+H)⁺; ¹H NMR (400 MHz, DMSO-d₆): δ 8.21 (brs, 1H), 7.69 (d, 2H), 7.32 (m, 5H), 6.92 (brs, 2H), 5.03 (s, 2H), 3.96 (d, 2H), 3.53 (m, 1H), 2.91 (brs, 2H), 2.70 (s, 3H), 1.86 (d, 2H), 1.36 (m, 2H).

### 2) 4-[(2-chloro-acetyl)-(4-methylaminocarbonyl-phenyl)-amino]-piperidine-1-carboxylic acid benzyl ester

4-(4-methylaminocarbonyl-phenylamino)-piperidine-1-carboxylic acid benzyl ester hydrogen chloride (8.90 g, 22.0 mmol), pyridine (5.0 mL, 61.7 mmol) were added to ethyl acetate (107 mL). The solution was cooled to 15° C. Chloroacetyl chloride (2.63 mL, 33.1 mmol) was added slowly to keep the temperature below 25° C. The mixture was stirred at 22° C. for 3 hours. Water 925 mL) was added and the mixture was stirred for 10 min. The organic layer was separated, washed with 2N aq. hydrochloric acid, sat. aq. sodium bicarbonate and brine, and dried over magnesium sulfate. The organic layer was concentrated to dryness to give 4-[(2-chloro-acetyl)-(4-methylaminocarbonyl-phenyl)-amino]-piperidine-1-carboxylic acid benzyl ester (9.8 g) as light brown oil in 100% yield. LC-MS APCl (m/z) 445 (M+H)⁺; ¹H NMR (400 MHz, CDCl₃): δ 7.83 (d, 2H), 7.26 (m, 5H), 7.15 (d, 2H), 6.57 (d, 1H), 5.00 (brs, 2H), 4.72 (m, 1H), 4.17 (m, 2H), 3.63 (s, 2H), 2.97 (d, 3H), 2.82 (m, 2H), 1.77 (m, 2H), 1.16 (m, 2H).

### 3) Benzyl 4-{5-[(methylamino)carbonyl]-2-oxo-2,3-dihydro-1H-indol-1-yl}piperidine-1-carboxylate

4-[(2-chloro-acetyl)-(4-methylaminocarbony-phenyl)-amino]-piperidine-1-carboxylic acid benzyl ester (4.5 g, 10.1 mmol), Pd(OAc)₂ (114 mg, 0.51 mmol) and 2-(di-t-butylphosphino)biphenyl (302 mg, 1.01 mmol) were added to 2-Methyl tetrahydrofuran (45 mL) and isopropanol (9 mL). The system was purged with nitrogen. Triethylamine (2.12 mL 15.2 mmol) was added. The mixture was heated to 65° C. and stirred at this temperature for 23 hours. The mixture was cooled to 22° C. and concentrated under vacuum. Dichloromethane (60 mL) was added. The mixture was washed with 1N aq. hydrochloric acid, sat. aq. sodium bicarbonate and brine. The mixture was stirred with Magnesium sulfate and Darco KB for 1 hour and filtered through Celite with dichloromethane washing. The combined filtrates were concentrated to dryness. To the residue, ethyl acetate (20 mL), water (15 mL) and methyl t-butyl ether (20 mL) were added. The slurry was stirred at 22° C. for 2 hours and filtered. The cake was washed with methyl t-butyl ether and dried under vacuum to give Benzyl 4-{5-[(methylamino)carbonyl]-2-oxo-2,3-dihydro-1H-indol-1-yl}piperidine-1-carboxylate (2.46 g) as an off-white powder in 60% yield.

### 1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium chloride

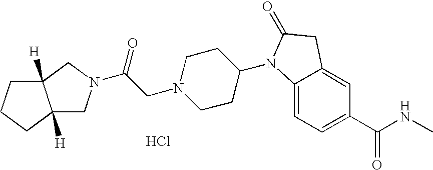

N-methyl-2-oxo-1-piperidin-4-ylindoline-5-carboxamide (0.18 g, 0.66 mmol) was suspended in 4 mL N,N-dimethylformamide. To the solution was added triethylamine (0.18 mL, 1.32 mmol) followed by (3aR,6aS)-2-(chloroacetyl)octahydrocyclopenta[c]pyrrole (0.12 g, 0.66 mmol) in 1 mL N,N-dimethylformamide. The reaction mixture was stirred at ambient temperature overnight. Partitioned solution between dichloromethane and 5% aq. sodium bicarbonate, dried organics with magnesium sulfate, filtered and concentrated in vacuo. The desired product was isolated by column chromatography eluting with a gradient of 0-25% methanol in ethyl acetate. The pure fractions were combined and concentrated, and subsequently dissolved in 5 mL methanol and 40 mL dichloromethane and bubbled hydrogen chloride (g) into the solution for 30 seconds. The resultant mixture was concentrated in vacuo. The concentrated product was diluted with 5 mL methanol and added diethyl ether slowly. A white solid precipitates. To this, was added a total of 100 mL diethyl ether. Stirred for 15 minutes then filtered off solids to yield title compound. (185 mg, 66%): APCl⁺ 425.20; Anal. HPLC Retention time=12.3 minutes (>99% pure). ¹H NMR (DMSO-d₆) δ 9.9 (s, 1H), 8.34 (m, 1H), 7.79 (m, 1H), 7.75 (s, 1H), 7.41 (d, 1H), 4.50 (m, 1H), 4.20 (m, 2H), 3.60-3.55 (m, 6H), 3.20-3.14 (m, 4H), 2.75-2.68 (m, 6H), 2.60 (m, 1H), 1.82-1.64 (m, 5H), 1.55 (m, 1H), 1.40 (m, 2H).

### 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide

A one-necked, 2-L, round-bottomed flask was equipped with an addition funnel and a magnetic stirbar, was charged with 4-(5-Methylcarbamoyl-2-oxo-2,3-dihydro-indol-1-yl)-piperidine-1-carboxylic acid benzyl ester (22.4 g, 82.0 mmol), and MeCN (230 mL). A solution of KHCO₃ (16.2 g, 164.1 mmol, 2.0 equiv.) and KI (2.7 g, 1.6 mmol, 0.2 equiv.) in H₂O (150 mL) was added. The reaction mixture was heated in a 60° C. oil bath with stirring. A solution of (3aR,6aS)-2-(chloroacetyl)octahydrocyclopenta[c]pyrrole in MeCN (50 mL) was added dropwise over 1 hr. After the addition, the mixture was stirred at the same temperature for 1 hr. The mixture was concentrated to remove most of the MeCN and a solid formed. The solid was filtered, washed with heptane, and dried at 45° C. vacuum oven over night to give a yellow solid, 33.93 g (97.5%).

The solid was suspended in acetone (100 mL) and stirred for 15 min. The solid was collected by filtration and dried to give a light yellow solid, 31.86 g (91.6%). Further purification by column chromatography (silica gel, EtOAc-MeOH/90:10) gave 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide; 23.64 g (68.0%), LC-MS APCl (m/z) 425 (M+H)⁺; ¹H NMR (300 MHz, DMSO-d₆): δ 8.25 (d, 1H), 7.72 (d, 1H), 7.69 (s, 1H), 7.10 (d, 1H), 4.03 (m, 1H), 3.60 (d, d, 1H), 3.54 (s, 2H), 3.42-3.35 (m, 3H), 3.13-3.10 (m, 2H), 3.08-3.03 (d, 1H), 2.92-2.89 (d, 2H), 2.71 (d, 3H), 2.63-2.45 (m, 2H), 2.33-2.29 (m, 2H), 2.28-2.15 (m, 2H), 1.74-1.14 (m, 8H).

### 1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium hydrogen sulfate

A one-necked, 1-L, round-bottomed flask was equipped with an addition funnel and a magnetic stirbar, was charged with 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide (23.8 g, 56.1 mmol), and MeCN (500 mL, 45 mg/mL). A solution of H₂SO₄ (5.5 M, 9.8 mL, 53.8 mmol, 0.96 equiv.) in H₂O (15 mL) and MeCN (45 mL) was added dropwise via the addition funnel. After the addition, the mixture was stirred at ambient temperature for 2 h. a gum formed first, and then turned to a solid. The solid was filtered, and washed with MeCN (2×50 mL), and dried at 45° C. vacuum oven over night, to give an off-white solid; 27.6 g (94.2%). LC-MS APCl (m/z) 425 (M+H)⁺.

### Preparation of methyl 2,5-difluoro-4-nitrobenzoate

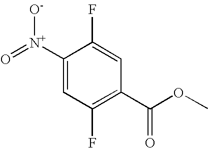

2,5-difluoro-4-nitrobenzoic acid (5.0 g, 25 mmol) was dissolved in 50 mL anhydrous methanol under a nitrogen atmosphere, added 0.5 mL concentrated sulfuric acid, then heated to reflux overnight. The reaction mixture was cooled to ambient temperature, concentrated in vacuo. Redissolved in diethyl ether (200 mL), washed with saturated sodium bicarbonate (3×100 mL), brine, dried with magnesium sulfate, filtered and concentrated to yield title compound. (5.06 g, 95%) APCl⁻ 217.29; Anal. HPLC Retention time=16.5 minutes (>99% pure).

### Preparation of di-tert-butyl[4-fluoro-5-(methoxycarbonyl)-2-nitrophenyl]malonate

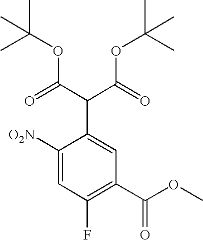

Di-t-butyl-malonate (7.0 mL, 32.2 mmol) was dissolved in 50 mL anhydrous N,N-dimethylformamide under a nitrogen atmosphere, cooled to 0° C., then added sodium hydride (60% dispersion in oil, 1.5 g, 37 mmol) in portions. Evolution of gas was noted. Stirred at 0° C. for 20 minutes, added 2,5-difluoro-4-nitro-benzoic acid methyl ester slowly (5.0 g, 23 mmol), dissolved in 15 mL anhydrous N,N-dimethylformamide. Removed cooling bath after 20 minutes then stirred at ambient temperature overnight. Partitioned between ethyl acetate and water, washing organics with more water, brine, dried with magnesium sulfate, filtered and concentrated in vacuo. The desired product was purified by column chromatography eluting with a gradient of 0-60% ethyl acetate in hexanes. The pure fractions were combined and concentrated to yield title compound. (6.15 g, 65%) APCl⁻ 413.36; Anal. HPLC Retention time=21.7 minutes.

### Preparation of di-tert-butyl[2-amino-4-fluoro-5-(methoxycarbonyl)phenyl]malonate

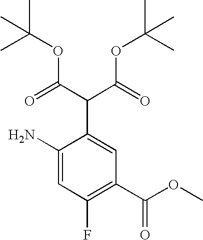

di-tert-butyl[4-fluoro-5-(methoxycarbonyl)-2-nitrophenyl]malonate (6.15 g, 14.9 mmol) was dissolved in methanol and tetrahydrofuran (1:1), added Raney Nickel. The solution was filtered and concentrated in vacuo to yield title compound. (5.5 g, 93%) APCl⁺ 384.13; Anal. HPLC Retention time=19.9 minutes.

### Preparation of di-tert-butyl[2-({1-[(benzyloxy)carbonyl]piperidin-4-yl}amino)-4-fluoro-5-(methoxycarbonyl)phenyl]malonate

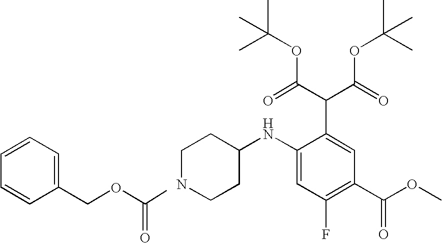

di-tert-butyl[2-amino-4-fluoro-5-(methoxycarbonyl)phenyl]malonate (5.5 g, 14 mmol) and N-benzyloxycarbonyl-4-piperidone (6.7 g, 28 mmol) were combined in 50 mL 1,2-dichloroethane under a nitrogen atmosphere, added acetic acid (4.1 mL, 71.7 mmol), followed by 0.5 equivalents of sodium triacetoxyborohydride (1.5 g, 7.2 mmol). The reaction mixture was stirred at ambient temperature for 4 hours. Added sodium triacetoxyborohydride (1.5 g, 7.2 mmol) and continued stirring at ambient temperature overnight. Added sodium triacetoxyborohydride (1.5 g, 7.2 mmol), stirred for 4 hours, added sodium triacetoxyborohydride (1.5 g, 7.2 mmol) and continued stirring at ambient temperature for 3 days. The reaction was quenched with the careful addition of 150 mL sat. sodium bicarbonate. Evolution of gas noted. The reaction mixture was treated with dichloromethane, and the organic layer was washed with brine and dried with magnesium sulfate, filtered. The filtrate was concentrated in vacuo. The desired product was purified by column chromatography eluting with a gradient of 20-100% ethyl acetate in hexanes. The desired fractions were combined and concentrated to yield title compound. (7.35 g, 78%) APCl⁺ 601.25; Anal. HPLC Retention time=23.2 minutes.

### Preparation of methyl 1-{1-[(benzyloxy)carbonyl]piperidin-4-yl}-6-fluoro-2-oxoindoline-5-carboxylate

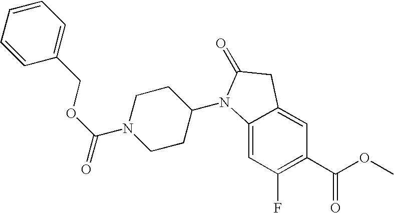

di-tert-butyl[2-({1-[(benzyloxy)carbonyl]piperidin-4-yl}amino)-4-fluoro-5-(methoxycarbonyl)phenyl]malonate (7.35 g, 12.2 mmol) was dissolved in 75 mL toluene, added p-toluenesulfonic acid monohydrate (0.47 g, 2.4 mmol) and refluxed the solution for 2 hours. The solution was cooled and concentrated in vacuo. The desired product was purified by column chromatography eluting with a gradient of 20-100% ethyl acetate in hexanes. Combined and concentrated pure fractions to yield title compound (4.46 g, 74%): APCl⁺ 427.11; Anal. HPLC Retention time=18.7 minutes (86% pure).

### Preparation of 1-(1-[(benzyloxy)carbonyl]piperidin-4-yl)-6-fluoro-2-oxoindoline-5-carboxylic acid

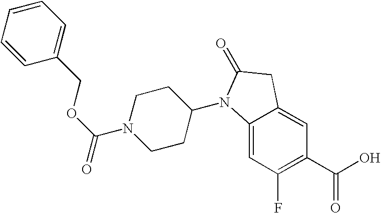

methyl 1-{1-[(benzyloxy)carbonyl]piperidin-4-yl}-6-fluoro-2-oxoindoline-5-carboxylate (1.5 g, 3.5 mmol) was suspended in 50 mL methanol, heated to 80° C. which caused solids to dissolve, added 2M aq. sodium hydroxide and stirred at 80° C. for 1.5 hours. The solution was cooled to ambient temperature, acidified with 1M aqueous hydrogen chloride extracted 3 times with dichloromethane, dried combined organics with magnesium sulfate, filtered and concentrated to yield title compound. (1.9 g, quant. yield) APCl⁺ 413.13; Anal. HPLC Retention time=16.7 minutes.

### Preparation of benzyl 4-{6-fluoro-5-[(methylamino)carbonyl]-2-oxo-2,3-dihydro-1H-indol-1-yl}piperidine-1-carboxylate

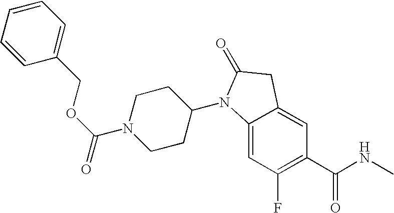

1-{1-[(benzyloxy)carbonyl]piperidin-4-yl}-6-fluoro-2-oxoindoline-5-carboxylic acid (1.4 g, 3.5 mmol) was dissolved in 20 mL anhydrous dichloromethane under a nitrogen atmosphere, added 1-hydroxybenzotriazole hydrate (0.80 g, 5.3 mmol) and N-(3-dimethylaminopropyl)-N′-ethylcarbodiimide hydrochloride (1.0 g, 5.3 mmol), stirred for 10 minutes then added 2M methylamine in tetrahydrofuran solution (7.0 mL, 14 mmol). The reaction mixture was stirred at ambient temperature for 3 days. Diluted with 100 mL dichloromethane, washed with sat. sodium bicarbonate (3×50 mL), 1M aqueous hydrogen chloride (3×50 mL), brine, dried with magnesium sulfate, filtered and concentrated in vacuo. The desired product was purified by column chromatography eluting with a gradient of 0-25% methanol in ethyl acetate. The fractions were combined and concentrated to yield title compound. (0.57 g, 38%) APCl⁺ 426.13; Anal. HPLC Retention time=16.4 minutes.

### Preparation of 6-fluoro-N-methyl-2-oxo-1-piperidin-4-ylindoline-5-carboxamide

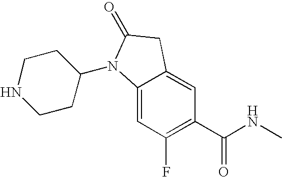

benzyl 4-{6-fluoro-5-[(methylamino)carbonyl]-2-oxo-2,3-dihydro-1H-indol-1-yl}piperidine-1-carboxylate (0.57 g, 1.34 mmol) was dissolved in methanol (25 mL) and tetrahydrofuran (25 mL) and added 10% palladium on carbon (0.1 g) and shaken for 20 hours. The solution was filtered and concentrated to yield title compound. (0.39 g, quantitative): APCl⁺ 292.17; Anal. HPLC Retention time=10.2 minutes.

### EXAMPLE NO. 21

### 4-{6-fluoro-5-[(methylamino)carbonyl]-2-oxo-2,3-dihydro-1H-indol-1-yl}-1-{2-[(3aR,6aS)-hexahydrocyclopenta[c]pyrrol-2(1H)-yl]-2-oxoethyl}piperidinium chloride

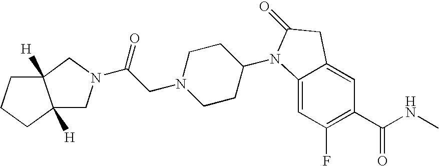

6-fluoro-N-methyl-2-oxo-1-piperidin-4-ylindoline-5-carboxamide (0.18 g, 0.66 mmol) was dissolved in 4 mL N,N-dimethylformamide, added triethylamine (0.18 mL, 1.32 mmol) followed by (3aR,6aS)-2-(chloroacetyl)octahydrocyclopenta[c]pyrrole (0.12 g, 0.66 mmol) dissolved in 1 mL N,N-dimethylformamide. The reaction mixture was stirred at ambient temperature for 4 hours. Partitioned solution between dichloromethane and 5% aqueous sodium bicarbonate solution, dried organics with magnesium sulfate, filtered and concentrated in vacuo. The desired product was purified by column chromatography eluting with a gradient of 0-25% methanol in ethyl acetate. The pure fractions were combined and concentrated, and subsequently re-dissolved in 5 mL methanol and 40 mL dichloromethane and bubbled hydrogen chloride (g) into the solution for 30 seconds. The reaction mixture was concentrated in vacuo to yield title compound (213 mg, 71%): APCl⁺ 443.25; Anal. HPLC Retention time=12.5 minutes (97% pure). ¹H NMR (DMSO-d₆) δ 9.9 (s, 1H), 8.01 (m, 1H), 7.53 (d, 1H), 7.39 (d, 1H), 4.50 (m, 1H), 4.18 (m, 2H), 3.75 (m, br, 4H), 3.58 (m, 2H), 3.20-3.14 (m, 4H), 2.76-2.68 (m, 6H), 2.60 (m, 1H), 1.82-1.64 (m, 5H), 1.55 (m, 1H), 1.40 (m, 2H).

### EXAMPLE NO. 22

### 1-[2-(4,4-dimethylpiperidin-1-yl)-2-oxoethyl]-4-{6-fluoro-5-[(methylamino)carbonyl]-2-oxo-2,3-dihydro-1H-indol-1-yl}piperidinium chloride

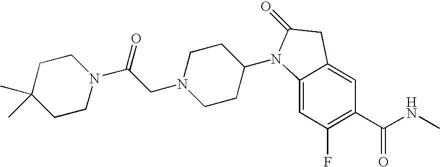

6-fluoro-N-methyl-2-oxo-1-piperidin-4-ylindoline-5-carboxamide (0.19 g, 0.66 mmol) was dissolved in 4 mL N,N-dimethylformamide, added triethylamine (0.18 mL, 1.32 mmol) followed by the 2-chloro-1-(4,4-dimethyl-piperidin-1-yl)-ethanone (0.125 g, 0.66 mmol) dissolved in 1 mL N,N-dimethylformamide. The reaction mixture was stirred at ambient temperature for 4 hours. Partitioned solution between dichloromethane and 5% aq. sodium bicarbonate, dried organics with magnesium sulfate, filtered and concentrated in vacuo. The desired product was purified by column chromatography eluting with a gradient of 0-25% methanol in ethyl acetate. The pure fractions were combined and concentrated. The resultant material was diluted in 5 mL methanol and 40 mL dichloromethane and was bubbled hydrogen chloride (g) into the solution for 30 seconds. The reaction mixture was concentrated in vacuo, and subsequently dissolved in acetonitrile/water mixture and lyophilized to yield title compound as the hydrochloride salt. (152 mg, 50%): APCl⁺ 445.26; Anal. HPLC Retention time=13.1 minutes (97% pure).

### EXAMPLE NO. 23

### 1-[2-(4,4-dimethylpiperidin-1-yl)-2-oxoethyl]-4-{5-[(methylamino)carbonyl]-2-oxo-2,3-dihydro-1H-indol-1-yl}piperidinium chloride

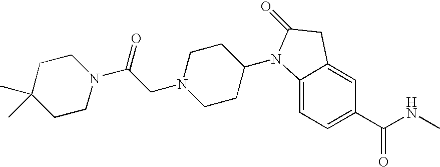

Synthesized in a similar manner to 1-{2-[(3aR,6aS)-hexahydrocyclopenta[c]pyrrol-2(1H)-yl]-2-oxoethyl}-4-{5-[(methylamino)carbonyl]-2-oxo-2,3-dihydro-1H-indol-1-yl}piperidinium chloride substituting 2-chloro-1-(4,4-dimethyl-piperidin-1-yl)-ethanone for (3aR,6aS)-2-(chloroacetyl)octahydrocyclopenta[c]pyrrole. (185 mg, 66%): APCl⁺ 427.25; Anal. HPLC Retention time=12.8 minutes (>99% pure). Benzyl 4-methylenepiperidine-1-carboxylate

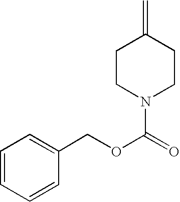

A slurry of (Methyl)triphenylphosphonium Bromide (57.4 g, 161 mmol) in anhydrous tetrahydrofuran (THF, 250 ml) was cooled in an ice bath and treated slowly with a potassium tert-butoxide solution in THF (18 g, 161 mmol). The mixture was stirred for 30 minutes and then allowed to warm to room temperature for 30 minutes. The reaction was cooled again in an ice bath and treated with a solution of 1-(Benzyloxycarbonyl)-4-piperidinone (25 g, 110 mmol) in THF (65 ml). The mixture was allowed to warm to room temperature and was stirred over the weekend. The reaction was concentrated, resuspended in water (100 ml) and hexanes (100 ml). The layers were separated and the aqueous layer was extracted with hexanes (2×100 ml) and heptane (1×100 ml). The combined organics were washed with brine, dried over sodium sulfate, filtered, and concentrated to give a yellow oil which was purified by silica gel chromatography to yield an oil/liquid (26 g, 100%). Experimental Data; LCMS 90% H₂O, Retention time 2.41 min., Parent C₁₄H₁₇NO₂, MW 231.3; Found APCl⁺ 232.3 (M+1); ¹H NMR (CDCl₃) δ 7.4-7.2 (m, 5H), 5.12 (s, 2H), 4.72 (s, 2H), 3.48 (m, 4H), 2.18 (m, 4H).

### Benzyl 6-azaspiro[2.5]octane-6-carboxylate

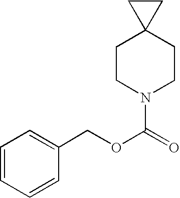

To a solution of diethylzinc (95.1 ml, 1.0M in heptane, 95.1 mmol) in anhydrous dichloromethane (DCM, 100 ml) cooled in an ice bath, was added trifluoroacetic acid (7.4 ml, 95.1 mmol) in DCM (15 ml) via syringe very slowly. The mixture was stirred cold for 30 minutes. The reaction was treated with diuodomethane (7.8 ml, 95.1 mmol) in DCM (5 ml) and stirred cold for 30 minutes. The mixture was treated with 4-methylene-piperidine-1-carboxylic acid benzyl ester (11.0 g, 47.6 mmol) in DCM (6 ml). The mixture was allowed to warm to room temperature and stirred overnight. The reaction was diluted with DCM (150 ml) and poured into saturated sodium bicarbonate solution (300 ml) forming a heavy white precipitate. The solid was removed by filtering and washing the solid with DCM (200 ml). The aqueous layer was removed and the organics were washed with brine, dried over sodium sulfate, filtered, and concentrated to a dark yellow syrup. The material was purified by silica gel chromatography (EtOAc/Heptane, 0 to 20%) to give colorless oil (10.639, 91%). Experimental Data; Parent C₁₅H₁₉NO₂, MW 245.32; MS Found APCl⁺ 246.2 (M+1); ¹H NMR (CDCl₃) δ 7.4-7.2 (m, 5H), 5.10 (s, 2H), 3.43 (m, 4H), 1.30 (m, 4H), 0.30 (s, 4H).

### 6-azoniaspiro[2.5]octane chloride

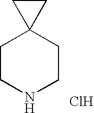

Benzyl 6-azaspiro[2.5]octane-6-carboxylate (10.6 g, 43.2 mmol) was dissolved in THF (190 ml) in a reactor vessel. 10% Palladium on carbon (3 g) was added and the reactor was purged/pressurized with hydrogen gas (500 psi). Reactor was sealed and shaken for 33.7 hours. Mixture was filtered with additional THF. Hydrogen chloride gas was bubbled through the solution with stirring for 15 minutes. The mixture was concentrated to near dryness, resuspended in diethyl ether, filtered, and the filtrate washed with diethyl ether. The solid was dried in a vacuum oven at 65° C. (5.0 g, 78%). Experimental Data; Parent C₇H₁₃N, MW 111.18; ¹H NMR (DMSO-d₆) δ 9.10 (br, 1H), 2.95 (m, 4H), 1.50 (m, 4H), 0.35 (s, 4H).

### 6-(chloroacetyl)-6-azaspiro[2.5]octane

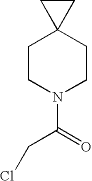

Dissolved 6-azoniaspiro[2.5]octane chloride (1.19, 7.5 mmol) and 2N sodium carbonate (9.3 ml, 19 mmol) in DCM (10 ml) while stirring in an ice bath. Slowly added 2-chloroacetyl chloride (0.712 ml, 9 mmol) dropwise and stirred cold for 30 minutes. The reaction mixture was stirred at room temperature for 1 h, and subsequently was diluted with dichloromethane (DCM, 20 ml) and water (20 ml). Separated layers and extracted aqueous layer twice with DCM. Combined organics were washed with brine, dried over sodium sulfate, filtered, and concentrated to yield pale-yellow oil (1.39 g, 99%). Experimental Data; LCMS 50% H₂O, Retention time 1.916 min., Parent C₉H₁₄ClNO, MW 187.08; Found APCl⁺ 188.1 (M+1); ¹H NMR (CDCl₃) δ 4.05 (s, 2H), 3.7-3.4 (m, 4H), 1.50-1.30 (m, 4H), 0.35 (s, 4H).

### EXAMPLE NO. 24

### 1-{-[2-(6-azaspiro[2.5]oct-6-yl)-2-oxoethyl]piperidin-4-yl}-N-methyl-2-oxoindoline-5-carboxamide

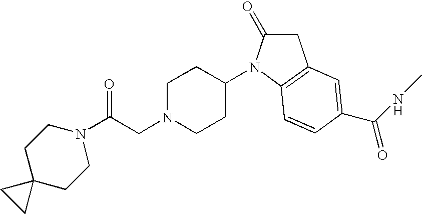

Ice cold suspension of N-methyl-2-oxo-1-piperidin-4-ylindoline-5-carboxamide (7.74 g, 28.3 mmol) in DMF (150 ml) was treated with triethylamine (7.52 ml, 53.93 mmol) and stirred for 5 minutes. The mixture was treated dropwise with a solution of 6-(chloroacetyl)-6-azaspiro[2.5]octane (5.06 g, 28.3 mmol) in DMF (30 ml) and stirred overnight at room temperature. The reaction was concentrated and the residue was taken up in DCM and washed with a 5% sodium bicarbonate solution, water (4×), and brine. The organics were dried over sodium sulfate, filtered, and concentrated to yield foam. The material was purified by chromatography on silica gel eluting with MeOH/EtOAc (0 to 35%) to yield a white solid (5.32 g, 47%). Experimental Data; LCMS 90% H₂O, Retention time 2.21 min., Parent C₂₄H₃₂N₄O₃, MW 424.54; Found APCl⁺ 425.4 (M+1); ¹H NMR (DMSO-d₆) δ 8.25 (m, 1H), 7.74-7.68 (m, 2H), 7.09 (m, 1H), 4.06-4.00 (m, 1H), 3.54-3.50 (m, 4H), 3.45-3.42 (m, 2H), 3.13 (m, 2H), 2.90 (d, 2H), 2.71 (d, 3H), 2.36-2.26 (m, 2H), 2.15-2.09 (m, 2H), 1.56-1.53 (m, 2H), 1.36 (m, 2H), 1.21 (m, 2H), 0.30 (d, 4H).

### EXAMPLE NO. 25

### 1-[2-6-azaspiro[2.5]oct-6-yl)-2-oxoethyl]-4-{5-[(methylamino)carbonyl]-2-oxo-2,3-dihydro-1H-indol-1-yl}piperidinium chloride

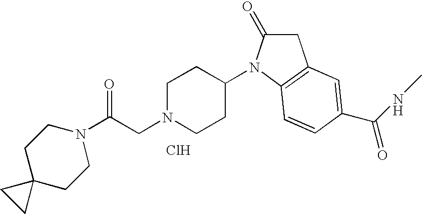

Ice cold solution of 1-{1-[2-(6-azaspiro[2.5]oct-6-yl)-2-oxoethyl]piperidin-4-yl}-N-methyl-2-oxoindoline-5-carboxamide (5.32 g, 12.5 mmol) in DCM (400 ml) was treated with HCl gas for 20 minutes. The reaction was sealed and stirred at room temperature for 60 minutes. The reaction was concentrated. The residue was taken up in MeOH (anhydrous, 25 ml) and treated with diethyl ether (170 ml). The mixture was refrigerated for 1 hour and the solid was collected by filtration. The solid was washed with cold diethyl ether and dried in a vacuum oven overnight at −60° C. to yield white solid (5.49 g, 95%). Experimental Data; LCMS 90% H₂O, Retention time 2.28 min., Parent C₂₄H₃₂N₄O₃, MW 424.54; Found APCl⁺ 425.2 (M+1) APCl− 423.1 (M−1); ¹H NMR (DMSO-d₆) δ 9.89 (br, 1H), 8.40 (m, 1H), 7.84-7.78 (m, 2H), 7.48-7.46 (m, 1H), 4.53 (m, 1H), 4.35 (d, 2H), 3.62-3.55 (m, 6H), 3.38-3.35 (m, 2H), 3.28-3.19 (m, 2H), 2.83-2.76 (m, 5H), 1.85-1.82 (m, 2H), 1.42-1.30 (m, 4H), 0.39-0.34 (m, 4H).

### Preparation of tert-butyl (4-{6-fluoro-5-[(methylamino)carbonyl]-2-oxo-2,3-dihydro-1H-benzimidazol-1-yl}piperidin-1-yl)acetate

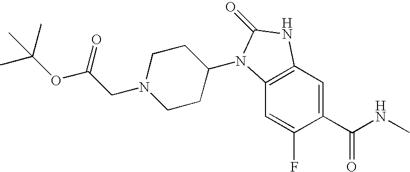

4-{6-fluoro-5-[(methylamino)carbonyl]-2-oxo-2,3-dihydro-1H-benzimidazol-1-yl}piperidinium trifluoroacetate (5.58 g, 19.1 mmol) was dissolved in 40 mL N,N-dimethylformamide, cooled to 0° C., added triethylamine (13.3 mL, 95.5 mmol) followed by the dropwise addition of t-butylbromoacetate (2.96 mL, 20.1 mmol). Allowed solution to slowly warm to ambient temperature, then stirred 13 hours. The solution was partitioned between ethyl acetate and sat. sodium bicarbonate, extracted aqueous once more with ethyl acetate, washed combined organics with water, brine, dried with magnesium sulfate, filtered and concentrated in vacuo. The desired product was purified by column chromatography eluting with a gradient of 0-30% methanol in ethyl acetate. The desired fractions were combined and concentrated pure fractions to yield title compound. (5.16 g, 66%) APCl⁺ 407.2; Anal. HPLC Retention time=12.2 minutes (>99% pure).

### Preparation of (4-{6-fluoro-5-[(methylamino)carbonyl]-2-oxo-2,3-dihydro-1H-benzimidazol-1-yl}piperidin-1-yl)acetic acid

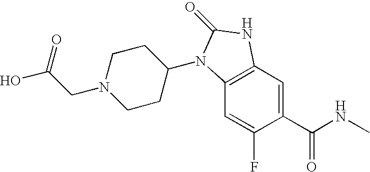

tert-butyl (4-{6-fluoro-5-[(methylamino)carbonyl]-2-oxo-2,3-dihydro-1H-benzimidazol-1-yl}piperidin-1-yl)acetate (5.1 g, 13 mmol) was dissolved in 20 mL dichloromethane and 40 mL trifluoroacetic acid and stirred at ambient temperature overnight. The reaction mixture was concentrated in vacuo, and the remaining TFA was evaporated with dichloromethane as an azeotrope to yield title compound as the trifluoroacetate salt. (8.82 g, quantitative): APCl⁺ 351.1; Anal. HPLC Retention time=9.9 minutes (98.7% pure).

### EXAMPLE NO. 26

### 1-[2-(4,4-dimethylpiperidin-1-yl)-2-oxoethyl]-4-{6-fluoro-5-[(methylamino)carbonyl]-2-oxo-2,3-dihydro-1H-benzimidazol-1-yl}piperidinium chloride

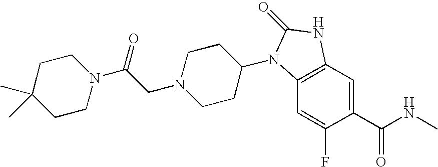

(4-{6-fluoro-5-[(methylamino)carbonyl]-2-oxo-2,3-dihydro-1H-benzimidazol-1-yl}piperidin-1-yl)acetic acid (1.2 g, 1.8 mmol) was dissolved in 10 mL N,N-dimethylformamide, added triethylamine (1.2 mL, 8.8 mmol), 4,4-dimethyl-piperidine hydrochloride (0.2 g, 1.8 mmol), followed by benzotriazol-1-yl-oxy-tripyrrolidinophosphonium hexafluorophosphate (1.1 g, 2.1 mmol). The reaction mixture was stirred at ambient temperature for 3 hours. The solution was diluted with ethyl acetate, washed with a sat. sodium bicarbonate solution, brine, dried with magnesium sulfate, filtered and concentrated. The desired product was purified by column chromatography eluting with a gradient of 0-30% methanol in ethyl acetate. The desired fractions were combined and concentrated containing product. The product was converted to the hydrochloride salt using Amberlite IRA-400 (Cl) resin. The desired product was lyophilized to yield title compound. (0.45 g, 57%): APCl⁺ 446.2; Anal. HPLC Retention time=12.8 minutes (>99% pure).

### EXAMPLE NO. 27

### 1-{1-[2-(4-ethyl-4-methylpiperidin-1-yl)-2-oxoethyl]piperidin-4-yl}-6-fluoro-N-methyl-2-oxo-2,3-dihydro-1H-benzimidazole-5-carboxamide

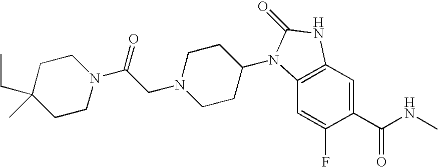

Synthesized as described in Example 26 substituting 4-ethyl-4-methyl-piperidine for 4,4-dimethyl-piperidine hydrochloride. (450 mg, 61%) APCl⁺ 460.2; Anal. HPLC Retention time=13.6 minutes (98% pure).

### EXAMPLE NO. 28

### 1-{1-[2-(6-azaspiro[2.5]oct-6-yl)-2-oxoethyl]piperidin-4-yl}-6-fluoro-N-methyl-2-oxo-2,3-dihydro-1H-benzimidazole-5-carboxamide

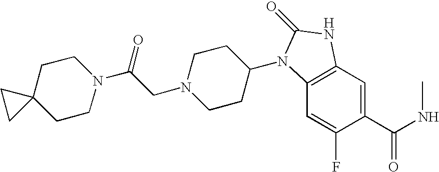

Prepared as described in Example 26 substituting 6-aza-spiro[2.5]octane for 4,4-dimethyl-piperidine hydrochloride. (13 mg, 3%) APCl⁺ 444.2.

### EXAMPLE NO. 29

### 6-fluoro-N-methyl-1-{1-[2-(4-methylonepiperidin-1-yl)-2-oxoethyl]piperidin-4-yl}-2-oxo-2,3-dihydro-1H-benzimidazole-5-carboxamide

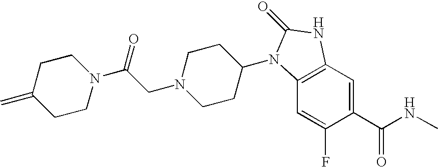

Synthesized as described in Example 26 with an exception of substituting 4-methylene-piperidine for 4,4-dimethyl-piperidine hydrochloride. (52 mg, 12%) APCl⁺ 430.2.

### EXAMPLE NO. 30

### 1-{1-[2-(3-azabicyclo[3.2.2]non-3-yl)-2-oxoethyl]piperidin-4-yl}-6-fluoro-N-methyl-2-oxo-2,3-dihydro-1H-benzimidazole-5-carboxamide

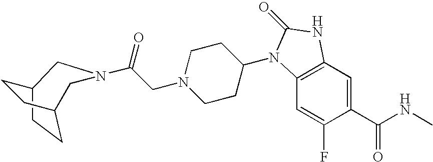

Synthesized as described in Example 26 with an exception of substituting 4-methylene-piperidine for 4,4-dimethyl-piperidine hydrochloride. (52 mg, 12%) APCl⁺ 430.2; Anal. HPLC Retention time=12.6 minutes (98% pure).

### EXAMPLE NO. 31

### 6-fluoro-1-(1-{2-[(3aR,6aS)-hexahydrocyclopenta[c]pyrrol-2(1H)-yl]-2-oxoethyl}piperidin-4-yl)-N-methyl-2-oxo-2,3-dihydro-1H-benzimidazole-5-carboxamide

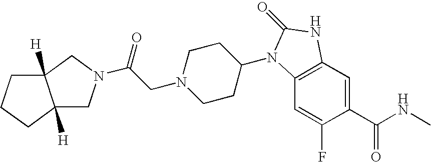

Synthesized as described in Example 26 with an exception of substituting octahydro-cyclopenta[c]pyrrole for 4,4-dimethyl-piperidine hydrochloride. (55 mg, 17%) APCl⁺ 444.16; Anal. HPLC Retention time=12.1 minutes (>99% pure).

### EXAMPLE NO. 32

### 6-fluoro-1-{1-[2-(4-methoxy-4-methylpiperidin-1-yl)-2-oxoethyl]piperidin-4-yl}-N-methyl-2-oxo-2,3-dihydro-1H-benzimidazole-5-carboxamide

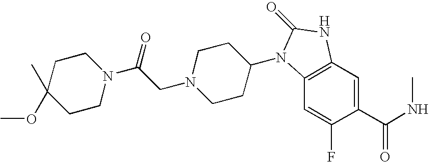

Synthesized as described in Example 26 with an exception of substituting 4-methoxy-4-methyl-piperidine for 4,4-dimethyl-piperidine hydrochloride. (49 mg, 6%) APCl⁺ 462.1; Anal. HPLC Retention time=11.5 minutes (98% pure).

### Preparation of tert-butyl 4-{5-[(ethylamino)carbonyl]-2-oxo-2,3-dihydro-1H-benzimidazol-1-yl}piperidine-1-carboxylate

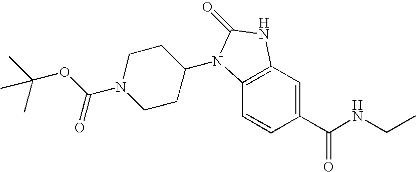

1-[1-(tert-butoxycarbonyl)piperidin-4-yl]-2-oxo-2,3-dihydro-1H-benzimidazole-5-carboxylic acid (6 g, 17 mmol) was suspended in 50 mL dichloromethane, added N-(3-dimethylaminopropyl)-N′-ethylcarbodiimide hydrochloride (4.8 g, 25 mmol) and 1-hydroxybenzotriazole hydrate (3.8 g, 25 mmol), then stirred at ambient temperature for 20 minutes. To the reaction mixture was added 2M ethylamine in tetrahydrofuran (33 mL, 66 mmol) slowly, and was stirred at ambient temperature overnight. The solution was diluted with 100 mL dichloromethane, washed with 1M aq. hydrogen chloride (2×75 mL), sat. sodium bicarbonate (3×75 mL), brine, dried over magnesium sulfate, filtered and concentrated in vacuo. Precipitation was observed as treated with a mixture of ethyl acetate and hexanes. The solid was filtered and washed with 25% ethyl acetate in hexanes to yield the title compound. (5.6 g, 87%) APCl⁻ 387.2; Anal. HPLC Retention time=14.9 minutes (>99% pure).

### Preparation of 4-{5-[(ethylamino)carbonyl]-2-oxo-2,3-dihydro-1H-benzimidazol-1-yl}piperidinium trifluoroacetate

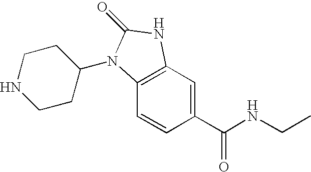

tert-butyl 4-{5-[(ethylamino)carbonyl]-2-oxo-2,3-dihydro-1H-benzimidazol-1-yl}piperidine-1-carboxylate (3.2 g, 8.2 mmol) was stirred in 30 mL dichloromethane and 30 mL trifluoroacetic acid at ambient temperature for 3 hours. The reaction mixture was concentrated in vacuo, which in turn was diluted and evaporated repeatedly with dichloromethane and chloroform to yield title compound as trifluoroacetate salt. (5.33 g, quant.) APCl⁺ 289.1; Anal. HPLC Retention time=10.1 minutes (>99% pure).

### Preparation of tert-butyl 4-{5-[(ethylamino)carbonyl]-3-methyl-2-oxo-2,3-dihydro-1H-benzimidazol-1-yl}piperidine-1-carboxylate

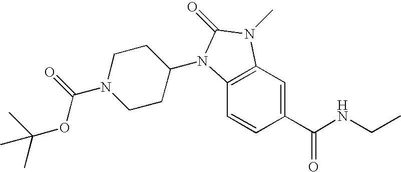

Tert-butyl 4-{5-[(ethylamino)carbonyl]-2-oxo-2,3-dihydro-1H-benzimidazol-1-yl}piperidine-1-carboxylate (0.4 g, 1.0 mmol) was dissolved in 5 mL N,N-dimethylformamide, added cesium carbonate (0.50 g, 1.5 mmol) followed by iodomethane (96 μL, 1.5 mmol). The reaction mixture was stirred at ambient temperature overnight. Partitioned between ethyl acetate and water, washed with water, brine, dried with magnesium sulfate, filtered and concentrated to yield title compound. (440 mg, quantitative) APCl⁺ 303.2 (minus t-butylcarboxyl group); Anal. HPLC Retention time=14.9 minutes (>99% pure).

### Preparation of 4-{5-[(ethylamino)carbonyl]-3-methyl-2-oxo-2,3-dihydro-1H-benzimidazol-1-yl}piperidinium trifluoroacetate

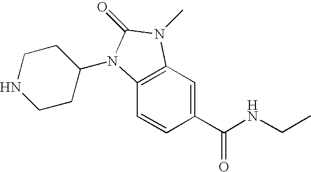

tert-butyl 4-{5-[(ethylamino)carbonyl]-3-methyl-2-oxo-2,3-dihydro-1H-benzimidazol-1-yl}piperidine-1-carboxylate (0.4 g, 1.0 mmol) was stirred in 10 mL dichloromethane and 10 mL trifluoroacetic acid at ambient temperature for 2 hours. The reaction mixture was concentrated in vacuo, and further concentrated by evaporating with dichloromethane to yield the title compound as trifluoroacetate salt. (quantitative yield) APCl⁺ 303.1; Anal. HPLC Retention time=10.1 minutes (>99% pure).

### EXAMPLE NO. 33

### 1-[2-(4,4-dimethylpiperidin-1-yl)-2-oxoethyl]-4-{5-[(ethylamino)carbonyl]-3-methyl-2-oxo-2,3-dihydro-1H-benzimidazol-1-yl}piperidinium chloride

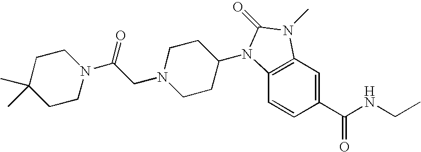

4-{5-[(ethylamino)carbonyl]-3-methyl-2-oxo-2,3-dihydro-1H-benzimidazol-1-yl}piperidinium trifluoroacetate (0.3 g, 1 mmol) was dissolved in 5 mL N,N-dimethylformamide, added triethylamine (0.7 mL, 5 mmol) followed by the dropwise addition of 2-chloro-1-(4,4-dimethyl-piperidin-1-yl)-ethanone (0.23 g, 1.2 mmol) which was dissolved in 2 mL N,N-dimethylformamide. The reaction mixture was stirred at ambient temperature overnight. Added 5% aq. sodium bicarbonate slowly and a precipitate forms. Slurried for 1 hour then filtered off solids. Redissolved in 5 mL methanol and 25 mL dichloromethane, cooled to 0° C., bubbled in anhydrous hydrogen chloride (g) for 30 seconds, concentrated in vacuo to yield title compound as the hydrochloride salt. (310 mg, 68%) APCl⁺ 456.2; Anal. HPLC Retention time=13.2 minutes (95% pure).

### EXAMPLE NO. 34

### 1-[2-(4,4-dimethylpiperidin-1-yl)-2-oxoethyl]-4-{5-[(ethylamino)carbonyl]-2-oxo-2,3-dihydro-1H-benzimidazol-1-yl}piperidinium chloride

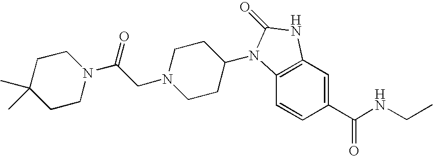

4-{5-[(ethylamino)carbonyl]-2-oxo-2,3-dihydro-1H-benzimidazol-1-yl}piperidinium trifluoroacetate (0.75 g, 2.6 mmol) was dissolved in 5 mL N,N-dimethylformamide, added triethylamine (0.82 mL, 5.9 mmol) followed by the dropwise addition of 2-chloro-1-(4,4-dimethyl-piperidin-1-yl)-ethanone (0.27 g, 1.4 mmol) which was dissolved in 2 mL N,N-dimethylformamide. The reaction mixture was stirred at ambient temperature overnight. Added 5% aqueous sodium bicarbonate slowly and a precipitate forms. Slurried for 1 hour then filtered off solids. The solid was dissolved in 5 mL methanol and 25 mL dichloromethane, cooled to 0° C., bubbled in anhydrous hydrogen chloride (g) for 30 seconds, subsequently concentrated in vacuo. This was diluted in 5 mL methanol and precipitated title compound with diethyl ether as the hydrochloride salt. (484 mg, 94%) APCl⁺ 442.2; Anal. HPLC Retention time=12.8 minutes (95.1% pure).

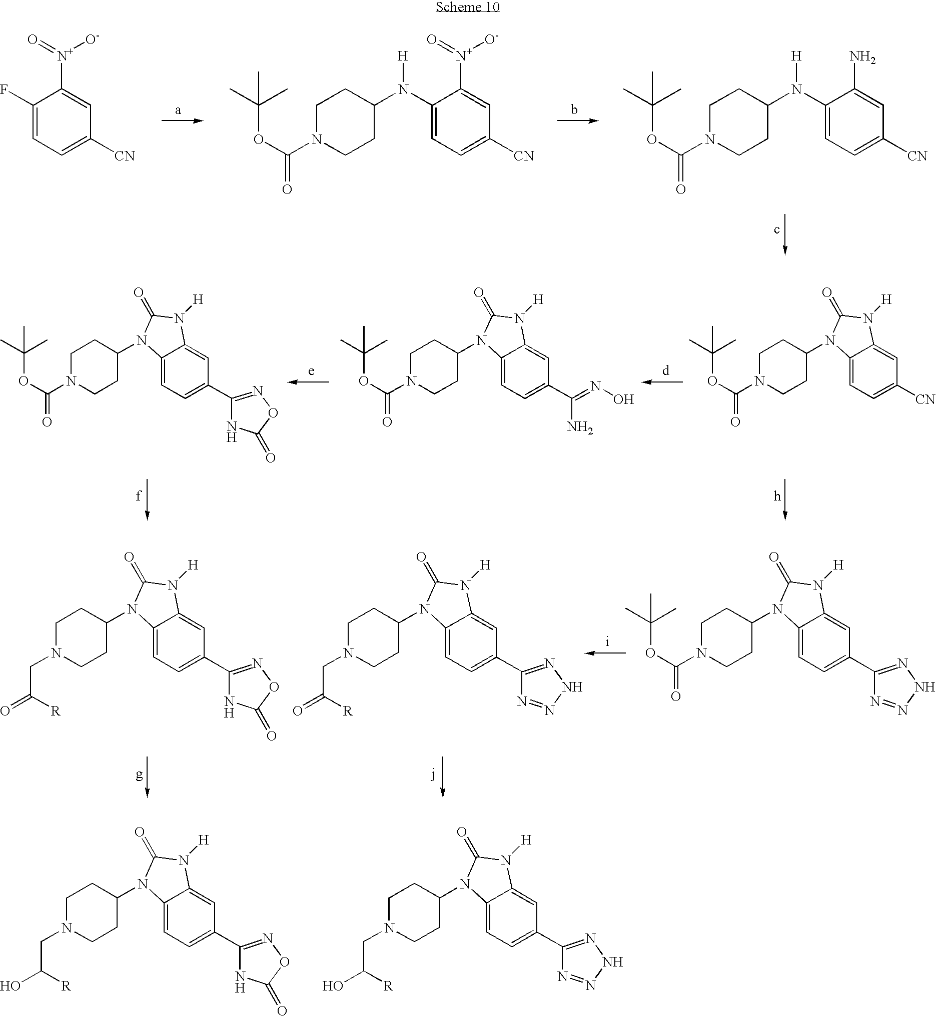

Reaction conditions: a) 4-amino N-1-Bocpiperidine, DIEA, DMF, rt, 3 days b) 10% Pd/C, EtOH, 50° C., 8 h, c) N,N′-carbonyldiimidazole (CDI), 4-(dimethylamino)pyridine (DMAP) 60° C., 2.5 hours, THF, d) MeOH, N,N-diisoproprylethylamine (DIEA), hydroxylamine hydrochloride (NH₂OH HCl), 60° C. overnight. e) DMF, pyridine, 2-ethylhexylchloroformate 0° C., 2 hour, f) 30% (v/v) TFA/DCM, then. DMF, 0° C., added triethylamine (TEA), 4′-chlorophenacyl bromide, g) NaBH₄, EtOH, h) dibutyltin oxide, trimethylsilylazide, 110° C., overnight, i) 30% (v/v) TFA/DCM, then DMF, 0° C., TEA, RC(O)CH₂Cl j) NaBH₄, EtOH.

### Preparation of tert-butyl 4-[(4-cyano-2-nitrophenyl)amino]piperidine-1-carboxylatetert-butyl 4-[(4-cyano-2-nitrophenyl)amino]piperidine-1-carboxylate

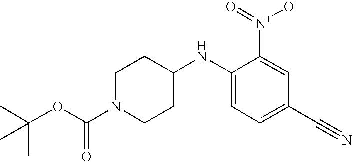

4-Amino-piperidine-1-carboxylic acid tert-butyl ester (6.15 g, 30.7 mmol), 4-fluoro-3-nitro-benzonitrile (5.0 g, 30 mmol), and N,N-diisoproprylethylamine (10.5 mL, 60.2 mmol) were combined in 40 mL N,N-dimethylformamide and stirred at ambient temperature for 3 days. The reaction was then diluted with 100 mL water and 250 mL ethyl acetate. Seperated layers, washed with 0.5M aq. hydrogen chloride (3×100 mL), then added about 100 mL tetrahydrofuran which caused the suspended solids to dissolve. Washed with brine (2×100 mL), dried with magnesium sulfate, filtered and concentrated to yield title compound. (10.55 g, quant. yield) APCl⁻ 346.1; Anal. HPLC Retention time=19.3 minutes.

### Preparation of tert-butyl 4-[(2-amino-4-cyanophenyl)amino]piperidine-1-carboxylate

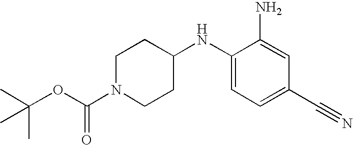

tert-butyl 4-[(4-cyano-2-nitrophenyl)amino]piperidine-1-carboxylatetert-butyl 4-[(4-cyano-2-nitrophenyl)amino]piperidine-1-carboxylate (8.7 g, 25 mmol) was suspended in 250 mL absolute ethanol, added cyclohexene (10.2 mL, 100 mmol) followed by 10% palladium on carbon (6.68 g, 6.3 mmol). The flask was placed in an oil bath at 50° C. for 8 hours. The heat was turned off and the reaction was stirred at ambient temperature overnight. The solution was filtered through a pad of Celite, washing with ethyl acetate. The reaction mixture was concentrated to yield title compound. (4.9 g, 62%) APCl⁻ 315.1; Anal. HPLC Retention time=15.5 minutes.

### Preparation of tert-butyl 4-(5-cyano-2-oxo-2,3-dihydro-1H-benzimidazol-1-yl)piperidine-1-carboxylate

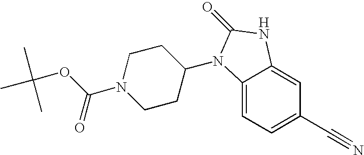

tert-butyl 4-[(2-amino-4-cyanophenyl)amino]piperidine-1-carboxylate (4.9 g, 15 mmol) was dissolved in 75 mL tetrahydrofuran, added N,N′-carbonyldiimidazole (5.0 g, 31 mmol) and 4-(dimethylamino)pyridine (0.19 g, 1.5 mmol) then heated to 60° C. for 2.5 hours. The solution was cooled to ambient temperature and allowed to sit over the weekend. Diluted with 100 mL ethyl acetate and another 50 mL tetrahydrofuran, washed with 1M aq. hydrogen chloride, brine, dried with magnesium sulfate, filtered and concentrated. Slurried in ethyl acetate, then filtered off solids, washing with cold ethyl acetate and hexanes to yield title compound. (4.08 g, 77%) APCl⁻ 341.1; Anal. HPLC Retention time=16.3 minutes.

### Preparation of tert-butyl 4-[2-oxo-5-(1H-tetrazol-5-yl)-2,3-dihydro-1H-benzimidazol-1-yl]piperidine-1-carboxylate

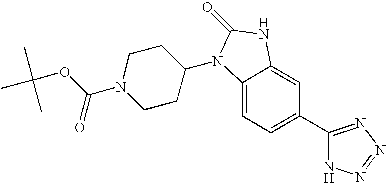

Tert-butyl 4-(5-cyano-2-oxo-2,3-dihydro-1H-benzimidazol-1-yl)piperidine-1-carboxylate (0.2 g, 0.58 mmol), dibutyltin oxide (15 mg, 0.058 mmol), and trimethylsilylazide (0.16 mL, 1.17 mmol) were combined in 6 mL of degassed, anhydrous toluene under a nitrogen atmosphere and heated to 110° C. overnight. HPLC shows the reaction is about 50% complete. Added another 2 eq of trimethylsilylazide (0.16 mL, 1.17 mmol) and heated overnight. The solution was cooled to ambient temperature, diluted with ethyl acetate, washed with 0.1% aqueous hydrogen chloride, brine, dried with magnesium sulfate, filtered and concentrated in vacuo. The concentrated reaction mixture was purified by column chromatography eluting it with a gradient of 0-35% methanol in ethyl acetate. The desired fractions were combined and concentrated to yield title compound. (110 mg, 49%) APCl⁻ 384.2; Anal. HPLC Retention time=14.6 minutes.

### EXAMPLE NO. 35

### 1-{1-[2-(4-chlorophenyl)-2-oxoethyl]piperidin-4-yl}-5-(1H-tetrazol-5-yl)-1,3-dihydro-2H-benzimidazol-2-one

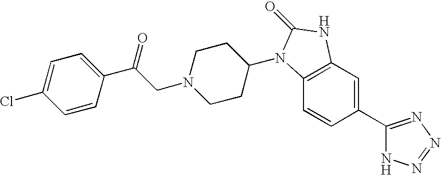

tert-butyl 4-[2-oxo-5-(1H-tetrazol-5-yl)-2,3-dihydro-1H-benzimidazol-1-yl]piperidine-1-carboxylate (0.19 g, 0.49 mmol) was stirred in 5 mL dichloromethane and 5 mL trifluoroacetic acid at ambient temperature for 2 hours, then concentrated in vacuo. Redissolved in 3 mL N,N-dimethylformamide, cooled to 0° C., added triethylamine (0.34 mL, 0.245 mmol) followed by the dropwise addition of 4′-chlorophenacyl bromide (0.11 g, 0.49 mmol) dissolved in 1 mL N,N-dimethylformamide. The solution was stirred at 0° C. for 1 hour. The solution was directly purified by prepratory HPLC. The desired fractions were collected and lyophilized to yield title compound. (91 mg, 42%) APCl⁻ 436.1, 438 (Cl pattern); Anal. HPLC Retention time=12.9 minutes (95% pure).

### EXAMPLE NO. 36

### 1-{1-[2-(4-chlorophenyl)-2-hydroxyethyl]piperidin-4-yl}-5-(1H-tetrazol-5-yl)-1,3-dihydro-2H-benzimidazol-2-one

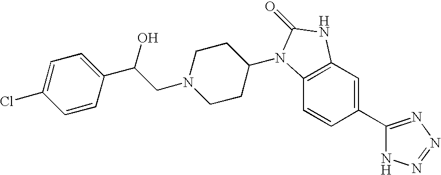

1-{1-[2-(4-chlorophenyl)-2-oxoethyl]piperidin-4-yl}-5-(1H-tetrazol-5-yl)-1,3-dihydro-2H-benzimidazol-2-one (52 mg, 0.119 mmol) was suspended in 1 mL ethanol, added sodium borohydride (4.4 mg, 0.119 mmol) and stirred at ambient temperature for 2 hours. Added 1 mL water and acidified with trifluoroacetic acid which caused solution to become homogeneous and gas was evolved. The resultant residue was purified by a preparatory HPLC. Combined and lyophilized pure fractions to yield title compound. (30 mg, 57%) APCl⁻ 438.1, 440.1 (Cl pattern); Anal. HPLC Retention time=12.9 minutes (>99% pure).

### tert-butyl 4-{5-[(Z)-amino(hydroxyimino)methyl]-2-oxo-2,3-dihydro-1H-benzimidazol-1-yl}piperidine-1-carboxylate

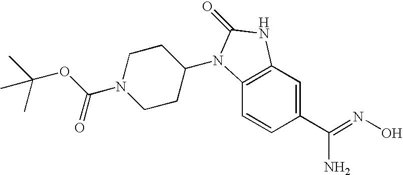

4-(5-Cyano-2-oxo-2,3-dihydro-benzoimidazol-1-yl)-piperidine-1-carboxylic acid tert-butyl ester (1.0 g, 2.9 mmol) was suspended in methanol (15 mL), added N,N-diisoproprylethylamine (1.5 mL, 8.8 mmol) and hydroxylamine hydrochloride (0.51 g, 7.3 mmol) and stirred 60° C. overnight. The solution was cooled to ambient temperature, partitioned between ethyl acetate and water, washed with water, brine, dried with magnesium sulfate, filtered and concentrated to yield title compound. (1 g, 91%) APCl⁻ 374.2; Anal. HPLC Retention time=12.8 minutes.

### Preparation of tert-butyl 4-[2-oxo-5-(5-oxo-4,5-dihydro-1,2,4-oxadiazol-3-yl)-2,3-dihydro-1H-benzimidazol-1-yl]piperidine-1-carboxylate

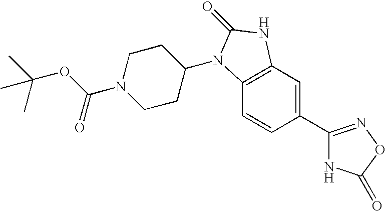

4-[5-(N-Hydroxycarbamimidoyl)-2-oxo-2,3-dihydro-benzoimidazol-1-yl]-piperidine-1-carboxylic acid tert-butyl ester (0.24 g, 0.64 mmol) was partially dissolved in 3 mL anhydrous N,N-dimethylformamide under an argon atmosphere, added pyridine (62 μL, 0.77 mmol), cooled to 0° C., then added 2-ethylhexylchloroformate (126 μL, 0.64 mmol) dropwise. Stirred at 0° C. for 1 hour. The solution had turned homogeneous by this time. The solution was diluted with ethyl acetate, washed with water, brine, dried with magnesium sulfate, filtered and concentrated. The reaction mixture was heated at reflux in 4 mL of o-xylene. The reaction starts off homogeneous and a precipitate forms within 15 minutes. The reaction mixture was heated at reflux for additional 3 hours. The flask was removed from heating bath and cooled to ambient temperature. Diluted with 15 mL hexanes and filtered to yield title compound. (0.19 g, 74%) APCl⁻ 400.2; Anal. HPLC Retention time=15.3 minutes.

### EXAMPLE NO. 37

### 1-{1-[2-(4-chlorophenyl)-2-oxoethyl]piperidin-4-yl}-5-(5-oxo-4,5-dihydro-1,2,4-oxadiazol-3-yl)-1,3-dihydro-2H-benzimidazol-2-one

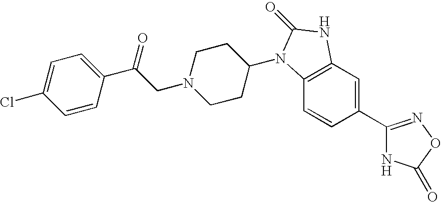

4-[2-Oxo-5-(5-oxo-4,5-dihydro-[1,2,4]oxadiazol-3-yl)-2,3-dihydro-benzoimidazol-1-yl]-piperidine-1-carboxylic acid tert-butyl ester (0.19 g, 0.47 mmol) was dissolved in 5 mL dichloromethane and 5 mL trifluoroacetic acid and stirred at ambient temperature for 1.5 hours. The solution was concentrated in vacuo. The mixture was dissolved in 3 mL N,N-dimethylformamide, cooled to 0° C., added triethylamine (0.33 mL, 2.35 mmol) followed by the dropwise addition of 4′-chlorophenacyl bromide (0.11 g, 0.47 mmol) in 1 mL N,N-dimethylformamide. The reaction mixture was stirred at 0° C. for 1 hour. The solution was purified by prepratory HPLC. The fractions were combined and lyophilized to yield the title compound. (75 mg, 35%) APCl⁺ 454.1, 456.1; Anal. HPLC Retention time=13.5 minutes (>99% pure).

### EXAMPLE NO. 38

### 1-{1-[2-(4-chlorophonyl)-2-hydroxyethyl]piperidin-4-yl}-5-(5-oxo-4,5-dihydro-1,2,4-oxadiazol-3-yl)-1,3-dihydro-2H-benzimidazol-2-one

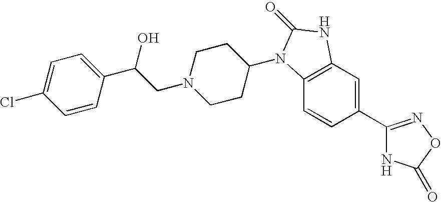

1-{1-[2-(4-Chloro-phenyl)-2-oxo-ethyl]-piperidin-4-yl}-5-(5-oxo-4,5-dihydro-[1,2,4]oxadiazol-3-yl)-1,3-dihydro-benzoimidazol-2-one (60 mg, 0.13 mmol) was dissolved in 2 mL ethanol, added sodium borohydride and stirred at ambient temperature for 3 hours. The reaction was quenched with water. The desired product was purified by a preparatory HPLC. The fractions were combined and lyophilized to yield title compound. (44 mg, 73%) APCl⁺; 456.1, 458.1; Anal. HPLC Retention time=13.5 minutes (>99% pure).

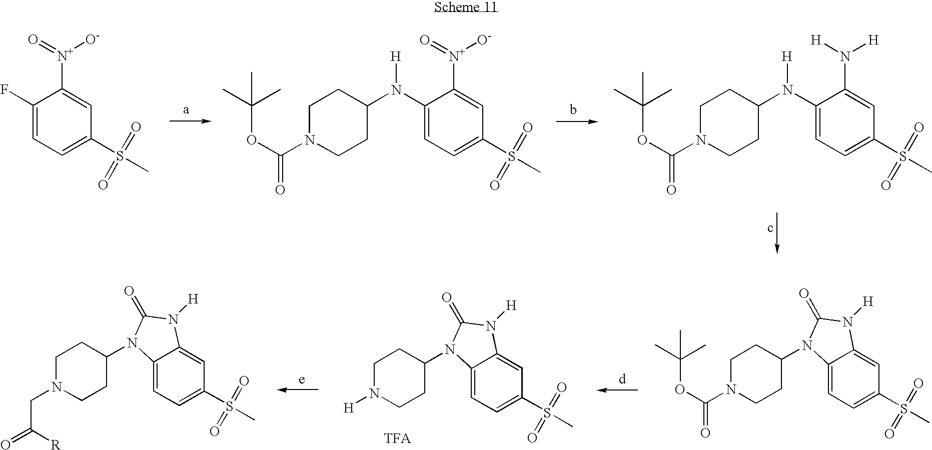

Reaction Conditions: a) 4-Amino-1-BocN-piperidine, DIEA, DMF, rt, overnight b) Raney Ni/THF c) CDl/DCM rt, d) TFA/DCM, rt, e) RC(O)CH₂Cl, DIEA, DMF rt,

### Preparation of 1-fluoro-4(methylsulfonyl)-2-nitrobenzene

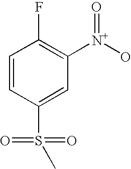

A mixture of concentrated sulfuric acid (16.0 ml, 290 mmole) and fuming nitric acid (8.0 ml, 176.4 mmole) was treated in small portions with 1-Fluoro-4-methanesulfonyl-benzene (5.00 g, 28.7 mmole) and stirred at room temperature for 2 hours. The reaction was poured carefully into crushed ice (140 g) forming a precipitate. The solid was collected by filtration and the solid was washed with water. A white solid was collected after drying in a vacuum oven (5.94 g, 94%). Experimental Data; LCMS 50% H₂O, Retention time 1.08 min., C₇H₆FNO₄S, MW 219.2; Found APCl⁻ 219.2(M+); ¹H NMR (CDCl₃) δ 8.65-8.63 (m, 1H), 8.22-8.18 (m, 1H), 7.53-7.43 (m, 1H), 3.10 (s, 3H).

### Preparation of tert-butyl 4-{[4-(methylsulfonyl)-2-nitrophenyl]amino}piperidine-1-carboxylate

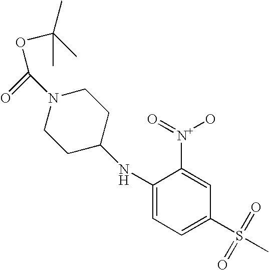

A solution of 1-Fluoro-4-methanesulfonyl-2-nitro-benzene (5.85 g (26.67 mmole) in dimethylformamide (22 ml) was treated with 4-Amino-piperidine-1-carboxylic acid tert-butyl ester (5.62 g, 27.2 mmole) and DMF (22 ml) followed by diisopropylethylamine (9.29 ml, 53.34 mmole). The mixture was stirred overnight at room temperature. The reaction was stirred at −70° C. for 1 hour and concentrated. The residue was taken up in diethyl ether and washed with water (2×100 ml), 0.5N HCl (2×100 ml), and brine. The organics were dried over Na₂SO₄, filtered, and concentrated to yield a yellow solid. The yellow solid was taken up in ethyl acetate (EtOAc) and washed again with water (4×) and brine. The organics were dried over Na₂SO₄, filtered, and concentrated to yield a yellow solid (10.45 g, 98%). Experimental Data; LCMS 50% H₂O, Retention time 2.32 min., C₁₇H₂₅N₃O₆S, MW 399.4; Found APCl⁺ 300.2(M+1-Boc) and APCl⁻ 398.3 (M+); ¹H NMR (CDCl₃) δ 8.75 (d, 1H), 8.41 (br, 1H), 7.85 (d, 1H), 6.96 (d, 1H), 4.05 (br, 2H) 3.71 (br, 1H), 3.02 (m, 5H), 2.04 (m, br, 2H), 1.55 (m, 2H), 1.44 (s, 9H).

Preparation tert-butyl 4-{[2-amino-4-(methylsulfonyl)phenyl]amino}piperidine-1-carboxylate

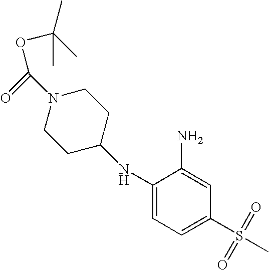

4-(4-Methanesulfonyl-2-nitro-phenylamino)-piperidine-1-carboxylic acid tert-butyl ester (10.37 g, 29.96 mmole) was reduced using 5 g of RaNi in tetrahydrofuran (THF, 150 ml) and concentrated to yield a pink-white solid (9.73 g, 101%). Experimental Data; LCMS 50% H₂O, Retention time 1.46 min., C₁₇H₂₇N₃O₄S, MW 369.4; Found APCl⁻ 369.2(M+), 270.2 (M+1-Boc); ¹H NMR (CDCl₃) δ 7.65-7.61 (m, br, 2H), 6.78 (d, 1H), 4.00 (d, br, 2H), 3.51 (br, 1H), 3.05-2.87 (m, 8H), 2.00 (br, 2H), 1.49-1.32 (m, 11H).

### tert-butyl 4-[5-(methylsulfonyl)-2-oxo-2,3-dihydro-1H-benzimidazol-1-yl]piperidine-1-carboxylate

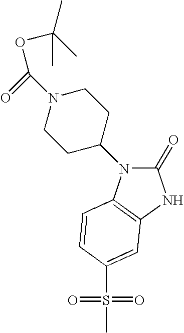

Treated a suspension of 4-(2-Amino-4-methanesulfonyl-phenylamino)-piperidine-1-carboxylic acid tert-butyl ester (9.70 g, 26.3 mmole) in THF (75 ml) with 1,1′-carbonyldiimidazole (CDI, 4.68 g, 28.9 mmole) and stirred overnight at room temperature. The reaction was diluted with EtOAc and washed with 0.5N HCl (3×40 ml)) and brine. The organics were dried over Na₂SO₄, filtered, and concentrated to yield a pink solid. Purification of a portion (6.0 g) on silica gel (Isco, 120 g column, EtOAc/Hex (50 to 100%)) gave pink solid (3.27 g (53%). Experimental Data; LCMS 70% H₂O, Retention time 2.48 min., C₁₈H₂₅N₃O₅S, MW 395.4; Found APCl⁺ 296.2 (M+1-Boc) and APCl⁻ 394.3 (M−1); ¹H NMR (DMSO-d₆) δ 11.31 (s, 1H), 7.53-7.51 (m, 1H), 7.43-7.37 (m, 2H), 4.35 (m, 1H), 4.10-4.00 (br, 2H), 3.11 (s, 3H), 2.82 (br, 2H), 2.20-2.10 (m, 2H), 1.70-1.60 (br, 2H), 1.38 (s, 9H).

### 5-(methylsulfonyl)-1-piperidin-4-yl-1,3-dihydro-2H-benzimidazol-2-one trifluoroacetate salt

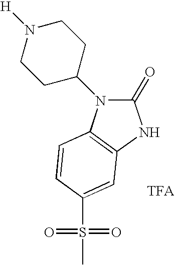

Solution of 4-(5-Methanesulfonyl-2-oxo-2,3-dihydro-benzoimidazol-1-yl)-piperidine-1-carboxylic acid tert-butyl ester (0.70 g, 1.77 mmole) in 20% TFA/DCM (25 ml) was stirred at room temperature for 2 hours. Reaction was concentrated. Residue was taken up in DCM and concentrated again (3×) to yield a solid (1.07 g, ˜2.7 equivalents of TFA). Experimental Data; LCMS 98% H₂O, Retention time 1.93 min., Parent C₁₃H₁₇N₃O₃S, MW 295.4; Found APCl⁺ 296.2 (M+1) and APCl⁻ 294.3 (M−1)); ¹H NMR (DMSO-d₆) δ 11.38 (s, 1H), 8.61 (m, br, 1H), 8.38 (m, br, 1H), 7.60-7.57 (m, 1H), 7.47-7.41 (m, 2H), 4.54 (m, br, 1H), 3.41-3.30 (m, 2H), 3.13-2.95 (m, 5H), 2.54-2.45 (m, 2H), 1.84 (d, br, 2H).

### EXAMPLE NO. 39

### 1-{1-[2-(4,4-dimethylpiperidin-1-yl)-2-oxoethyl]piperidin-4-yl}-5-(methylsulfonyl)-1,3-dihydro-2H-benzimidazol-2-one

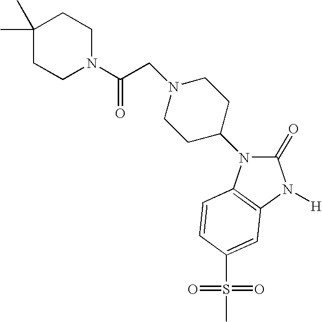

Ice cold solution of 5-Methanesulfonyl-1-piperidin-4-yl-1,3-dihydro-benzoimidazol-2-one trifluoroacetate salt (1.079, assumed to be 1.77 mmole) in DMF (10 ml) was treated with triethylamine (1.23 ml, 8.85 mmole) and stirred for 5 minutes. Treated dropwise with 2-Chloro-1-(4,4-dimethyl-piperidin-1-yl)-ethanone (0.34 g, 1.77 mmole) in DMF (2 ml) and stirred overnight at room temperature. Concentrated slightly and partitioned between DCM and 5% NaHCO₃. Aqueous layer was extracted with DCM. Combined organics were washed with water (3×) and brine. Dried over Na₂SO₄, filtered, and concentrated to yield a white solid that was dried in a vacuum oven at ˜50° C. for 2 hours (0.72 g, 90%). Experimental Data; LCMS 90% H₂O, Retention time 2.37 min., C₂₂H₃₂N₄O₄S, MW 448.2; Found APCl⁺ 449.2 (M+1) and APCl⁻ 447.4 (M−1)); ¹H NMR (DMSO-d₆) δ 11.30 (s, 1H), 7.54-7.52 (m, 1H), 7.38-7.35 (m, 2H), 4.15 (m, br, 1H), 3.46 (m, br, 2H), 3.39 (m, br, 2H), 3.13-3.11 (m, 5H), 2.90 (m, br, 2H), 2.30 (m, br, 2H), 2.08 (m, br, 2H), 1.63 (m, br, 2H), 1.33 (m, br, 2H), 1.18 (m, br, 2H), 0.91 (s, 6H)

CHN Calc.: C, 58.91; H, 7.19; N, 12.49

Found: C, 58.73; H, 7.34; N, 12.12

### EXAMPLE NO. 40

### 1-{1-[2-(4,4-dimethylpiperidin-1-yl)-2-oxoethyl]piperidin-4-yl}-5-(methylsulfonyl)-1,3-dihydro-2H-benzimidazol-2-one hydrochloride

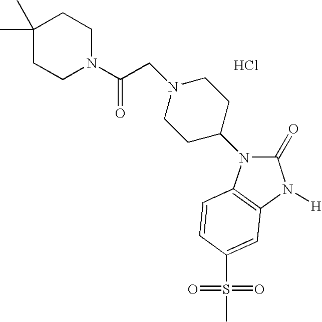

Solution of 1-{1-[2-(4,4-Dimethyl-piperidin-1-yl)-2-oxo-ethyl]-piperidin-4-yl}-5-methanesulfonyl-1,3-dihydro-benzoimidazol-2-one (0.52 g, 1.16 mmole) in DCM (25 ml) and MeOH (1 ml) was treated with HCl_{(g)} for 10 minutes. Sealed reaction and stirred at room temperature for 2 hours. Reaction was concentrated. The residue was treated with MeOH (1.5 ml) and diethyl ether (7 ml) and refrigerated for 1 hour. The solid was collected by filtration and washed solid with cold diethyl ether. The solid was dried in a vacuum oven overnight at −50° C. to yield a white solid (0.50 g, 89%). Experimental Data; LCMS 90% H₂O, Retention time 2.36 min., Parent C₂₂H₃₂N₄O₄S, MW 448.2; Found APCl⁺ 449.2 (M+1) and APCl⁻ 447.4 (M−1)); ¹H NMR (DMSO-d₆) δ 11.49 (s, 1H), 9.84 (br, 1H), 7.66-7.57 (m, 2H), 7.43 (s, 1H), 4.55 (m, br, 1H), 4.29 (s, 2H), 3.60 (m, br, 2H), 3.47 (m, 2H), 3.21 (m, br, 2H), 3.13 (s, 3H), 2.73 (m, br, 2H), 1.88 (m br, 2H), 1.32 (m, br, 2H), 1.25 (m, br, 2H), 0.92 (s, 6H)

CHN Calc. (1.05 HCl, 0.75H₂O): C, 52.81; H, 6.96; N, 11.20; total Cl, 7.44

Found: C, 52.50; H, 7.26; N, 10.85; total Cl, 7.27

### EXAMPLE NO. 41

### 1-[1-(2-cyclohexyl-2-oxoethyl)piperidin-4-yl]-5-(methylsulfonyl)-1,3-dihydro-2H-benzimidazol-2-one

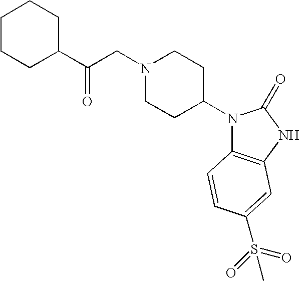

To a solution of 5-Methanesulfonyl-1-piperidin-4-yl-1,3-dihydro-benzoimidazol-2-one trifluoroacetate salt (0.372 g, 1.26 mmol) in DMF (5 mL), 2-Chloro-1-cyclohexyl-ethanone (0.243 g, 1.518 mmol), KI (50 mg) and TEA (2.63 mL, 18.979 mmol) were added sequentially at rt. The reaction mixture was heated to 80° C. and maintained over night. The reaction mixture was cooled to rt, diluted with ice cold water (15 mL) and extracted with EtOAc (2×20 mL). The organic layer was washed with brine solution (10 mL), dried over anhydrous sodium sulfate and concentrated to obtain the crude product. The crude was purified by preparative HPLC method to obtain the pure product as pale brown solid (35 mg, 6.6%). ¹HNMR (DMSO): δ 11.5(s, 1H), 9.8-10.04 (broad s, 1H), 7.4-7.78 (m, 3H), 4.4-4.75 (m, 2H), 3.4-3.62 (m, 3H), 3.1-3.3 (m, 4H), 2.6-2.86 (m, 2H), 1.7-2.08 (m, 5H) and 1.22-1.4 (m, 3H). Mass: (M+1) 420 calculated for C₂₁H₂₉N₃O₄S.

### EXAMPLE NO. 42

### 1-{1-[2-(4-methylcyclohexyl)-2-oxoethyl]piperidin-4-yl}-5-(methylsulfonyl)-1,3-dihydro-2H-benzimidazol-2-one

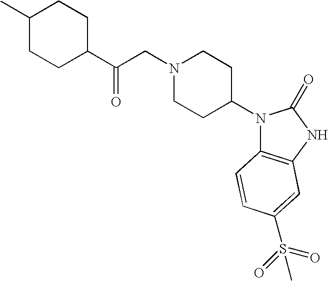

To a solution of 5-Methanesulfonyl-1-piperidin-4-yl-1,3-dihydro-benzoimidazol-2-one trifluoroacetate salt (0.5 g, 1.7 mmol) in dry DMF (10 mL), TEA (2.36 mL, 17 mmol) was added followed by the addition of 2-Chloro-1-(4-methyl-cyclohexyl)-ethanone (0.356 g, 2.04 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by preparative HPLC method to obtain the product as white solid (68 mg, 9.3%). IR (cm⁻¹): 3442, 3033, 2933, 1702, 1482, 1386, 1297 and 1139. ¹HNMR (CDCl₃): δ8.4-8.52 (s, 1H), 7.6-7.8 (m, 2H), 4.1 (s, 1H), 4.72 (m, 1H), 3.32 (m, 1H), 3.05 (s, 2H) and 0.9 (m, 3H). Mass: (M+1) 434 calculated for C₂₂H₃₁N₃O₄S.

### EXAMPLE NO. 43

### 1-{1-[2-(4-methoxycyclohexyl)-2-oxoethyl]piperidin-4-yl}-5-(methylsulfonyl)-1,3-dihydro-2H-benzimidazol-2-one

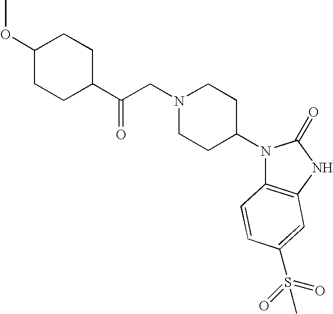

To a solution of 5-Methanesulfonyl-1-piperidin-4-yl-1,3-dihydro-benzoimidazol-2-one trifluoroacetate salt (0.5 g, 1.7 mmol) in dry DMF (10 mL), TEA (2.36 mL, 17 mmol) was added followed by the addition of 2-Chloro-1-(4-methoxy-cyclohexyl)-ethanone (0.388 g, 2.04 mmol) at rt and maintained for 2 h. The reaction mass was diluted with water (10 mL) and extracted with EtOAc (3×50 mL). The organic layer was washed with water (3×50 mL), brine solution (20 mL), dried over anhydrous sodium sulfate and concentrated to obtain the crude product. The crude was purified by preparative HPLC method to obtain the product as off-white solid (140 mg, 18.42%). IR (cm⁻¹): 3407, 2933, 2821, 1702, 1475, 1378, 1290 and 1120. ¹HNMR (CDCl₃): δ 11.38(s, 1H), 7.38-7.45 (m, 3H), 4.02-4.3 (m, 1H), 3.1-3.42 (m, 9H), 2.82-3.0 (m, 2H), 2.12-2.5 (m, 4H), 1.9-2.1 (m, 1H), 1.42-1.9 (m, 7H) and 1.02-1.32 (m, 1H). Mass: (M+1) 450 calculated for C₂₂H₃₁N₃O₅S.

### EXAMPLE NO. 44

### 1-{1-[2-(4-trifluoromethylcyclohexyl)-2-oxoethyl]piperidin-4-yl}-5-(methylsulfonyl)-1,3-dihydro-2H-benzimidazol-2-one

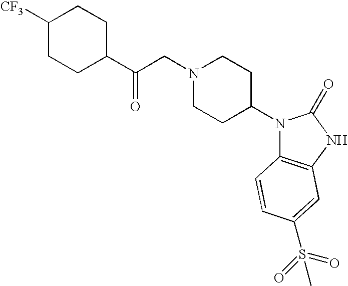

To a solution of 5-Methanesulfonyl-1-piperidin-4-yl-1,3-dihydro-benzoimidazol-2-one trifluoroacetate salt (0.374 g, 1.27 mmol) in dry DMF (5 mL), TEA (1.76 mL, 12.7 mmol) was added followed by the addition of 2-Chloro-1-(4-trifluoromethyl-cyclohexyl)-ethanone (0.35 g, 1.52 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by preparative HPLC method to obtain the product as brown solid (0.1 g, 16.8%). IR (cm⁻¹): 3434, 3253, 2944, 1710, 1617, 1479, 1294 and 1135.

¹HNMR (CDCl₃): δ 9.55(broad s, 1H), 7.7 (m, 2H), 7.45 (d, 1H), 4.3-4.52 (m, 1H), 3.32-3.55 (m, 2H), 3.0-3.2(m, 4H), 2.22-2.8 (m, 5H), 1.96-2.22 (m, 4H), 1.56-1.92 (m, 7H), 1.3-1.5 (m, 1H) and 1.1-1.3 (m, 1H). Mass: (M+1) 488 calculated for C₂₂H₂₈F₃N₃O₄S.

### EXAMPLE NO. 45

### 1-{1-[2-(4-tert-butylcyclohexyl)-2-oxoethyl]piperidin-4-yl}-5-(methylsulfonyl)-1,3-dihydro-2H-benzimidazol-2-one

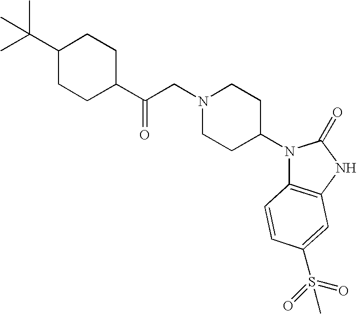

To a solution of 5-Methanesulfonyl-1-piperidin-4-yl-1,3-dihydro-benzoimidazol-2-one trifluoroacetate salt (0.4 g, 1.36 mmol) in dry DMF (5 mL), TEA (1.89 mL, 13.6 mmol) was added followed by the addition of 1-(4-tert-Butyl-cyclohexyl)-2-chloro-ethanon (0.35 g, 1.63 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by preparative HPLC method to obtain the product as pale brown solid (0.09 g, 14%). IR (cm⁻¹): 3311, 2940, 1710, 1479, 1301, 1139 and 1066. ¹HNMR (CDCl₃): δ 9.1-9.3 (m, 1H), 7.6-7.75 (m, 2H), 7.46 (d, 1H), 4.3-4.5 (m, 1H), 3.4 (s, 2H), 2.96-3.22 (m, 5H), 2.42-2.62 (m, 2H), 2.2-2.42 (m, 3H), 1.75-1.98 (m, 6H), 1.3-1.46 (m, 2H), 1.0-1.08 (m, 2H) and 0.82 (s, 9H). Mass: (M+1) 476 calculated for C₂₅H₃₇N₃O₄S.

### EXAMPLE NO. 46

### 1-(1-[2-(4 isopropylcyclohexyl)-2-oxoethyl]piperidin-4-yl)-5-(methylsulfonyl)-1,3-dihydro-2H-benzimidazol-2-one

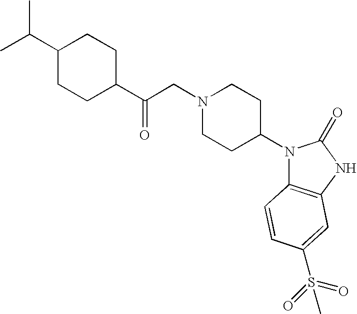

To a solution of 5-Methanesulfonyl-1-piperidin-4-yl-1,3-dihydro-benzoimidazol-2-one trifluoroacetate salt (0.37 g, 1.25 mmol) in dry DMF (5 mL), TEA (1.74 mL, 12.5 mmol) was added followed by the addition of 2-Chloro-1-(4-isopropyl-cyclohexyl)-ethanone (0.3 g, 1.5 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was washed with EtOAc (2×1 mL), filtered and dried to obtain the product as an off-white solid (0.24 g, 41.3%). IR (cm⁻¹): 3322, 2937, 1710, 1621, 1479, 1301 and 1139. ¹HNMR (CDCl₃): δ 9.0-9.7 (m, 2H), 7.6-7.8 (m, 2H), 7.5 (d, 1H), 4.32-4.52 (m, 1H), 3.4 (s, 2H), 3.0-3.22 (m, 5H), 2.45-2.65 (m, 2H), 2.22-2.42 (m, 3H), 1.74-1.98 (m, 6H), 1.6 (s, 5H), 1.3-1.52 (m, 3H), 0.98-1.14 (m, 3H) and 0.9 (d, 6H). Mass: (M+1) 462 calculated for C₂₄H₃₅N₃O₄S.

### EXAMPLE NO. 47

### 1-{1-[2-(4-methoxy-4-methylcyclohexyl)-2-oxoethyl]piperidin-4-yl}-5-(methylsulfonyl)-1,3-dihydro-2H-benzimidazol-2-one

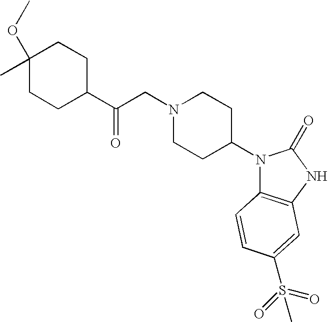

To a solution of 5-Methanesulfonyl-1-piperidin-4-yl-1,3-dihydro-benzoimidazol-2-one trifluoroacetate salt (0.29 g, 0.986 mmol) in dry DMF (5 mL), TEA (1.37 mL, 9.86 mmol) was added followed by the addition of 2-Chloro-1-(4-methoxy-4-methyl-cyclohexyl)-ethanone (0.24 g, 1.18 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by silica gel column using 3% MeOH in CHCl₃ as eluting solvent to obtain the product as dark brown liquid (200 mg, 44%). IR (cm⁻¹): 3436, 2935, 2819, 1697, 1643, 1477, 1295, 1133 and 1064. ¹HNMR (DMSO): δ 11.35(s, 1H), 7.6 (d, 1H), 7.45 (d, 1H), 4.2 (m, 1H), 3.12 (d, 5H), 2.92 (m, 2H), 2.72 (s, 1H), 2.1-2.45 (m, 3H), 1.32-1.85 (m, 8H), 1.258 (s, 1H) and 1.05(s, 3H). Mass: (M+1) 464 calculated for C₂₃H₃₃N₃O₅S.

### EXAMPLE NO. 48

### 1-{1-[2-(1-methylcyclohexyl)-2-oxoethyl]piperidin-4-yl}-5-(methylsulfonyl)-1,3-dihydro-2H-benzimidazol-2-one

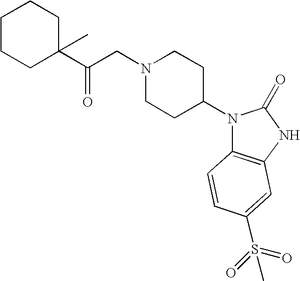

To a solution of 5-Methanesulfonyl-1-piperidin-4-yl-1,3-dihydro-benzoimidazol-2-one trifluoroacetate salt (0.35 g, 1.19 mmol) in dry DMF (5 mL), TEA (1.165 mL, 11.9 mmol) was added followed by the addition of 2-Chloro-1-(1-methyl-cyclohexyl)-ethanone (0.249 g, 1.42 mmol) at rt and maintained for 2 h. The reaction mass was diluted with water (10 mL) and extracted with EtOAc (3×20 mL). The organic layer was washed with water (3×20 mL), brine solution (20 mL), dried over anhydrous sodium sulfate and concentrated to obtain the crude product. The crude was purified by column chromatography over silica gel column using 2% MeOH in CHCl₃ as eluting solvent to obtain the product as pale brown solid (0.06 g, 11.6%). IR (cm⁻¹): 3434, 3311, 2933, 1706, 1621, 1475, 1297 and 1139. ¹HNMR (CDCl₃): δ 9.02-9.5 (m, 1H), 7.7 (m, 2H), 7.5 (d, 1H), 4.44 (m, 1H), 3.5 (m, 2H), 2.94-3.25 (m, 5H), 2.45-2.72 (m, 2H), 2.2-2.45 (m, 2H), 2.0 (d, 2H), 1.85 (d, 2H), 1.22-1.45 (m, 6H) and 1.12(s, 3H). Mass: (M+1) 434 calculated for C₂₂H₃₁N₃O₄S.

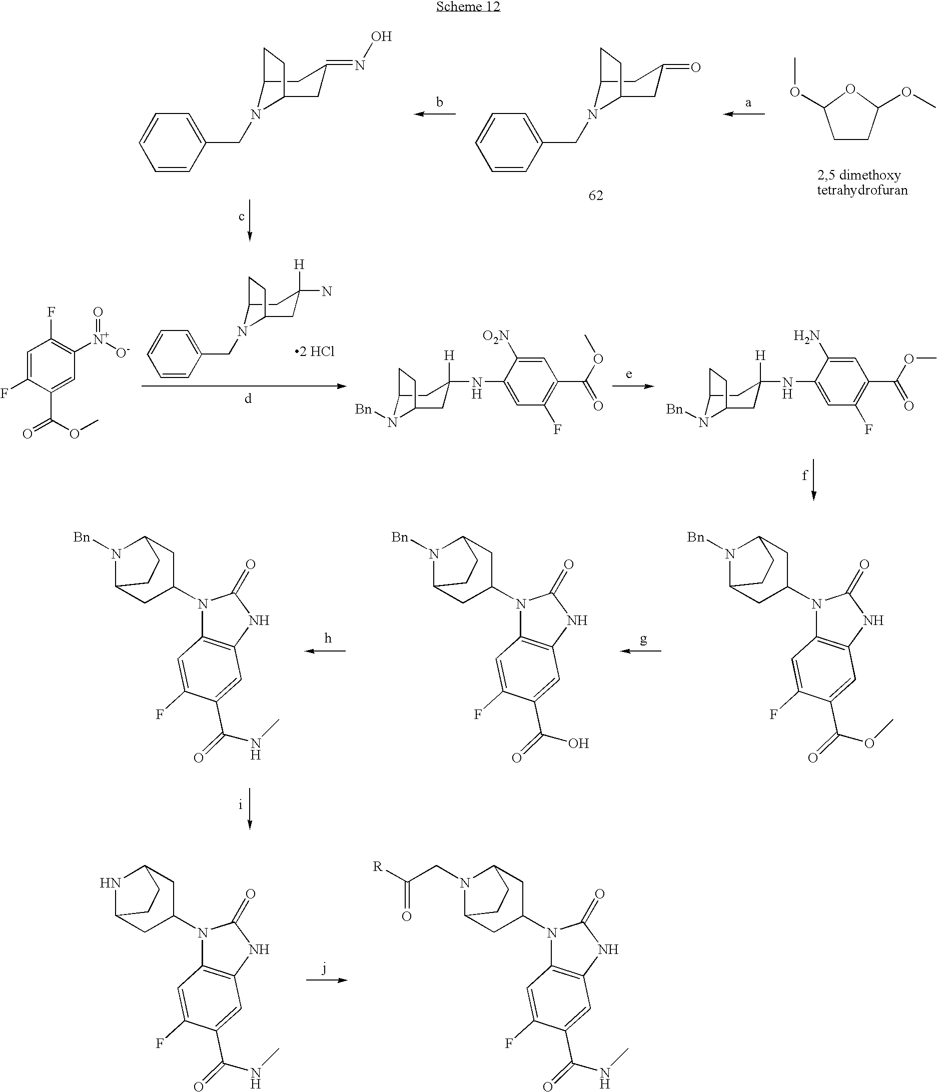

Reaction Conditions: a) i) 1N HCl, ii) NaOAc, Benzylamine, acetone di-carboxylic acid, b) NaOAc, NH2OH HCl, c) Na metal/EtOH Δ, HCl/MeOH, d) 64, DIEA, DMF, 0° C., e) RaNi, rt, f) CDl rt, g) LiOH, MeOH H₂O rt, g) CH₃NH₂, TEA, HBTU, h) NH₄COOH, Pd/C, MeOH, i) RC(O)CH2Cl, DMF, DIEA

### Preparation of 8-Benzyl-8-aza-bicyclo[3.2.1]octan-3-one

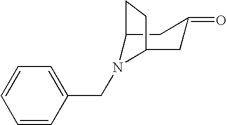

A solution of 2,5-dimethoxy tetrahydro furan (15 g, 113 mmol) in 1N HCl (250 mL) was heated to 70° C. and maintained for 1 h. The reaction mixture was cooled to 0° C., acetone di carboxylic acid (18.2 g, 125 mmol), conc.HCl (11 mL), sodium acetate (18.5 g, 136 mmol) and benzyl amine (14 mL, 125 mmol) were added sequentially, then the reaction mixture was allowed to rt slowly and maintained over night. The reaction mass was filtered through celite bed, aqueous layer of the filtrate was basified with NaOH solution and extracted with EtOAc (2×400 mL). The organic layer was dried over anhydrous sodium sulfate and concentrated to obtain the crude product as brown colored liquid (21.5 g, 88%).

¹HNMR (CDCl₃): δ 7.25-7.5 (m, 5H), 3.8 (s, 2H), 3.54 (s, 2H), 2.66-2.8 (m, 2H), 2.02-2.3 (m, 4H), 1.6-1.7 (m, 2H). Mass: (M+1) 216 calculated for C₁₄H₁₇NO.

### Preparation of 8-Benzyl-8-aza-bicyclo[3.2.1]octan-3-one oxime

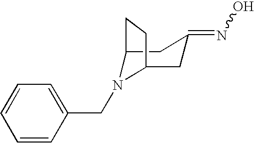

To a solution of 8-Benzyl-8-aza-bicyclo[3.2.1]octan-3-one (10.5 g, 48.8 mmol) in methanol, sodium acetate (26.5 g, 195.3 mmol) and NH₂OH.HCl (12.8 g, 185.5 mmol) were added sequentially at rt and maintained over night. The reaction mixture was concentrated to obtain a residue; residue was diluted with water (500 mL) and basified with 3N NaOH solution till pH was 8, resulted in the formation of white solid. The solid was filtered, washed with water (2×100 mL), hexane (2×100 mL) and dried to obtain crude product. The crude was recrystallised in ethanol to obtain product as white solid (7.4 g, 66%). ¹HNMR (CDCl₃): δ 8.5-8.7 (broad, 1H), 7.25-7.45 (m, 5H), 3.65 (s, 2H), 3.3-3.42 (m, 2H), 2.98 (d, 1H), 2.5-2.65 (m, 1H), 2.1-2.32 (m, 2H), 1.96-2.1 (m, 2H) and 1.5-1.7 (m, 2H). LC-MS APCl (m/z)=231 (M+1)⁺.

### Preparation of 8-Benzyl-8-aza-bicyclo[3.2.1]oct-3-ylamine

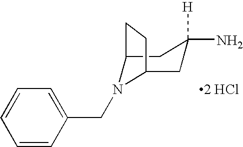

A solution of 8-Benzyl-8-aza-bicyclo[3.2.1]octan-3-one oxime (11 g, 47.8 mmol) in absolute ethanol (150 mL) was heated to 90° C., sodium metal (16 g) was added portion wise over a period of 12 h, then the reaction mixture was heated to 120° C. and maintained over night. The reaction mass was cooled to 50° C., quenched with methanol (75 mL) and stirred for 5 min. The reaction mass was cooled to 0° C., slowly acidified with methanolic HCl till pH was 2 and the resulted precipitate was concentrated to obtain the crude product as a hydrochloride salt (10.3 g, 100%). The crude was directly used in the next step without any purification. ¹HNMR (CDCl₃): δ 10.98-11.2 (m, 1H), 8.55-8.7 (m, 1H), 8.3-8.4 (m, 1H), 7.7-7.82 (m, 1H), 7.37-7.6 (m, 2H), 4.1-4.24 (m, 1H), 3.7-3.98 (m, 4H), 3.4-3.7 (m, 1H), 2.45-2.6 (m, 4H), 2.15-2.45 (m, 3H) and 1.8-2.1 (m, 2H). LC-MS APCl (m/z)=217 (M+1)⁺.

### Preparation of methyl 4-[(8-benzyl-8-azabicyclo[3.2.1]oct-3-yl)amino]-2-fluoro-5-nitrobenzoate

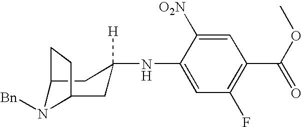

A solution of 2,4-difluoro-5-nitro-benzoic acid methyl ester (7.5 g, 34.7 mmol) in DMF (20 mL) was cooled to 0° C., DIEA (30 mL) was added followed by the addition of a solution of 8-Benzyl-8-aza-bicyclo[3.2.1]oct-3-ylamine 2HCl (10 g, 34.7 mmol) in DMF (20 mL). The reaction mixture was cooled to rt and maintained for 10 h. The reaction mass was diluted with DCM (1 L), the organic layer was washed with water (250 mL), brine solution, dried over anhydrous sodium sulfate and concentrated to obtain a residue; residue was washed with pet ether (250 mL), filtered and dried to obtain the crude product as yellow solid (7.7 g, 55%). ¹HNMR (CDCl₃): δ 8.9(d, 1H), 8.26 (d, 1H), 7.3-7.45 (m, 4H), 6.5 (d, 1H), 3.9 (s, 3H), 3.64-3.84 (m, 1H), 3.58 (s, 2H), 3.32 (s, 2H), 2.1-2.25 (m, 2H), 1.92-2.02 (m, 2H) and 1.7-1.82 (m, 4H). LC-MS APCl (m/z)=414 (M+1)⁺.

### Preparation of 5-Amino-4-(8-benzyl-8-aza-bicyclo[3.2.1]oct-3-ylamino)-2-fluoro-benzoic acid methyl ester

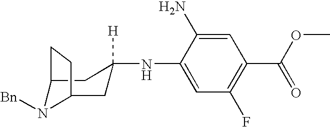

To a solution of methyl 4-[(8-benzyl-8-azabicyclo[3.2.1]oct-3-yl)amino]-2-fluoro-5-nitrobenzoate (10 g, 25 mmol) in methanol (100 mL), raney nickel (1.5 g) was added. The reaction mixture was arranged in parr shaker apparatus at rt and maintained for 12 h applying 55 Psi units. The reaction mass was filtered through celite bed, filtrate was concentrated to obtain the crude product as green solid (9.2 g, 96%). ¹HNMR (CDCl₃): δ 7.14-7.5 (m, 6H), 6.3 (d, 1H), 4.15 (m, 1H), 3.84 (s, 3H), 3.6 (s, 3H), 3.3 (s, 2H), 2.7-3.1 (m, 2H), 2.02-2.2 (m, 2H), 1.86-2.02 (m, 2H) and 1.5-1.8 (m, 4H). LC-MS APCl (m/z)=384 (M+1)⁺.

### Preparation of 1-(8-Benzyl-8-aza-bicyclo[3.2.1]oct-3-yl)-6-fluoro-2-oxo-2,3-dihydro-1H-benzoimidazole-5-carboxylic acid methyl ester

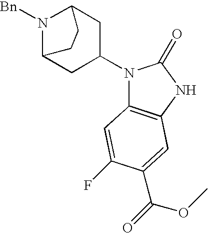

To a solution of 5-Amino-4-(8-benzyl-8-aza-bicyclo[3.2.1]oct-3-ylamino)-2-fluoro-benzoic acid methyl ester (5.5 g, 14.3 mmol) in dry THF (70 mL), CDI (11.6 g, 71.8 mmol) was added portion wise over a period of 10 min at rt and maintained for 14 h. The reaction mass was concentrated to obtain a residue; residue was diluted with DCM (500 mL). The organic layer was washed with water (250 mL), brine solution and concentrated to obtain the crude product as brown solid (4.7 g, 80%). ¹HNMR (DMSO): δ 10.1 (s, 1H), 7.68 (d, 1H), 7.25-7.55 (m, 5H), 7.12 (d, 1H), 4.62-4.84 (m, 1H), 3.92 (s, 3H), 3.65 (s, 2H), 3.38 (s, 2H), 2.36-2.55 (m, 2H), 2.04-2.25 (m, 2H), 1.8-1.95 (m, 2H) and 1.58-1.78 (m, 2H); LC-MS APCl (m/z)=410 (M+1)⁺.

### Preparation of 1-(8-Benzyl-8-aza-bicyclo[3.2.1]oct-3-yl)-6-fluoro-2-oxo-2,3-dihydro-1H-benzoimidazole-5-carboxylic acid

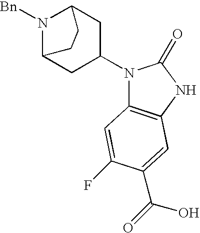

To a solution of 1-(8-Benzyl-8-aza-bicyclo[3.2.1]oct-3-yl)-6-fluoro-2-oxo-2,3-dihydro-1H-benzoimidazole-5-carboxylic acid methyl ester (6.3 g, 15.4 mmol) in methanol (40 mL), lithium hydroxide monohydrate (3.2 g, 77 mmol) was added followed by the addition of water (30 mL) at rt and maintained for 5 h. The reaction mixture was concentrated to obtain a residue; residue was diluted with DCM (500 mL) and again concentrated to obtain the crude product as brown solid (5.7 g, 94%). ¹HNMR (CDCl₃): δ 11.2-11.4 (broad s, 1H), 7.2-7.5 (m, 4H), 7.1 (d, 1H), 4.3-4.7 (m, 2H), 3.7 (s, 2H), 3.15 (m, 3H), 2.4 (s, 1H), 2.1 (m, 1H), 1.7-1.9(m, 1H), 1.42-1.6(m, 1H) and 1.1-1.2(m, 4H). LC-MS APCl (m/z)=395 (M+1)⁺.

### Preparation of 1-(8-Benzyl-8-aza-bicyclo[3.2.1]oct-3-yl)-6-fluoro-2-oxo-2,3-dihydro-1H-benzoimidazole-5-carboxylic acid methylamide

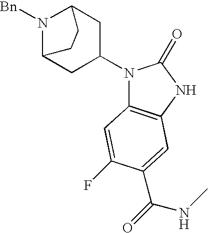

To a solution of 1-(8-Benzyl-8-aza-bicyclo[3.2.1]oct-3-yl)-6-fluoro-2-oxo-2,3-dihydro-1H-benzoimidazole-5-carboxylic acid (2 g, 5.0 mmol) in DMF (20 mL), HBTU (2 g, 2.7 mmol), MeNH₂ (2M solution in THF, 5.4 mL, 10 mmol) and TEA (6 mL) were added sequentially at rt and maintained for 12 h. The reaction mixture was extracted with DCM (250 mL), the organic layer was washed with water (250 mL), brine solution, dried and concentrated to obtain the crude product. The crude was purified by column chromatography over silica gel column using 2% methanol in CHCl₃ as eluting solvent to obtain the pure product as pale brown solid (1 g, 50%). ¹HNMR (CDCl₃): δ 8.7-9.12 (m, 1H), 7.8-7.85 (m, 1H), 7.3-7.5 (m, 6H), 7.1 (d, 1H), 6.7-6.85 (m, 1H), 4.6-4.86 (m, 2H), 3.65 (s, 3H), 3.36 (s, 3H), 2.42 (t, 3H) and 1.5-1.9 (m, 4H). LC-MS APCI (m/z)=409 (M+1)⁺.

### Preparation of 1-(8-Aza-bicyclo[3.2.1]oct-3-yl)-6-fluoro-2-oxo-2,3-dihydro-1H-benzoimidazole-5-carboxylic acid methylamide

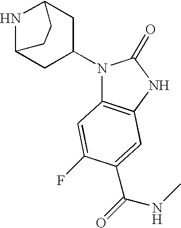

To a solution of 1-(8-Benzyl-8-aza-bicyclo[3.2.1]oct-3-yl)-6-fluoro-2-oxo-2,3-dihydro-1H-benzoimidazole-5-carboxylic acid methylamide (1 g, 2.4 mmol) in methanol (20 mL), ammonium formate (0.77 g, 12.2 mmol) and Pd/C (1 g) were added sequentially at rt. The reaction mixture was heated to 65° C. and maintained over night. The resulted reaction mass was filtered through celite bed, filtrate was concentrated to obtain the crude product as pale brown solid (0.7 g, 92%). ¹HNMR (DMSO): δ 7.9-8.1 (m, 1H), 7.7-7.9 (m, 1H), 7.25 (d, 1H), 4.55 (s, 1H), 4.04 (s, 1H), 3.22-3.5 (m, 4H), 3.15 (s, 1H), 1.65-2.12 (m, 5H) and 0.7-0.95 (m, 1H). LC-MS APCl (m/z)=319 (M+1)⁺.

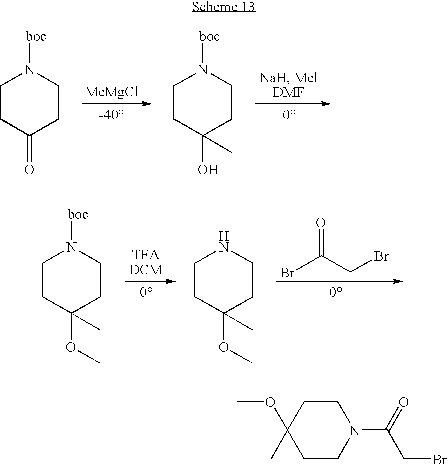

### Preparation of 4-Hydroxy-4-methyl-piperidine-1-carboxylic acid tert-butyl ester

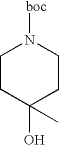

A solution of N-boc piperidin-4-one (3 g, 15 mmol) in dry THF (30 mL) was cooled to −40° C., 3M MeMgCl (21 mL, 60.24 mmol) was added slowly over 10 min. The reaction mixture was allowed to rt and maintained for 2 h. The reaction mixture was cooled to 0° C., sat. NH₄Cl was added and then was extracted with EtOAc (250 mL). The organic layer was washed with water (250 mL), brine solution, dried over anhydrous sodium sulfate and concentrated to obtain the crude product as yellow liquid (3 g, 93%).

¹HNMR (CDCl₃): δ 3.56-3.8 (m, 2H), 3.12-3.35 (m, 2H), 1.5-1.62 (m, 4H), 1.45 (s, 9H) and 1.26(s, 3H).

### Preparation of 4-Methoxy-4-methyl-piperidine-1-carboxylic acid tert-butyl ester

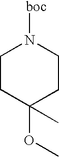

50% NaH (2.3 g, 96.7 mmol) was initially washed with dry THF, cooled to 0° C. and then a solution of 4-Hydroxy-4-methyl-piperidine-1-carboxylic acid tert-butyl ester (5.2 g, 24.18 mmol) in dry DMF (40 mL) was added. The resulted reaction mixture was allowed to rt, methyl iodide (3.1 mL, 43.3 mmol) was added and maintained for 7 h. The reaction mixture was quenched with water and then extracted with DCM (500 mL). The organic layer was washed with water (250 mL), brine solution and concentrated to obtain the crude product (as brown liquid (5 g, 90%). ¹HNMR (CDCl₃): δ 3.6-3.8 (m, 2H), 3.02-3.22 (m, 5H), 1.62-1.8 (m, 2H), 1.38-1.52 (m, 11H) and 1.1(s, 3H). LC-MS APCl (m/z)=130 (M+H-Boc)⁺.

### Preparation of 4-methoxy-4-methylpiperidine trifluoroacetate

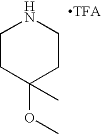

A solution of product 4-Methoxy-4-methyl-piperidine-1-carboxylic acid tert-butyl ester (5 g, 21.8 mmol) in DCM (20 mL) was cooled to 0° C., TFA (20 mL) was added. The reaction mixture was allowed to rt, maintained for 1 h and then was concentrated to obtain the crude product as brown liquid (2.8 g, 99.5%). ¹HNMR (CDCl₃): δ 8.8-9.44 (broad, 1H), 7.62-8.32 (m, 3H), 3.02-3.4 (m, 7H), 2.0 (d, 2H), 1.6-1.9 (m, 2H), 1.24 (s, 3H). LC-MS APCl (m/z)=130 (M+H)⁺.

### Preparation of 1-(bromoacetyl)-4-methoxy-4-methylpiperidine

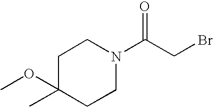

To a solution of 4-methoxy-4-methylpiperidine trifluoroacetate (2.8 g, 21.7 mmol) in DCM (40 mL), TEA (12 mL) was added. The reaction mixture was cooled to 0° C., bromo-acetyl bromide (3.1 mL, 1.0 mmol) was added, then was allowed to rt and maintained for 30 min. The reaction mass was extracted with EtOAc, the organic layer was washed with dil.HCl, sat.NaHCO₃ solution, water, brine solution and concentrated to obtain the crude product. The crude was purified by column chromatography over (60-120) mesh silica gel column using 25% EtOAc in hexane as eluting solvent to obtain the pure product as brown liquid (1.8 g, 33%). ¹HNMR (CDCl₃): δ 4.15-4.3 (m, 1H), 3.9 (q, 2H), 3.5-3.64 (m, 1H), 3.32-3.5 (m, 1H), 3.22 (s, 3H), 3.0 (t, 1H), 1.75-1.9 (m, 2H), 1.32-1.65 (m, 2H) and 1.2(s, 3H). LC-MS APCl (m/z)=252 (M+H)⁺.

### EXAMPLE NO. 49

### 6-fluoro-1-{8-[2-(4-methoxy-4-methylpiperidin-1-yl)-2-oxoethyl]-8-azabicyclo[3.2.1]oct-3-yl}-N-methyl-2-oxo-2,3-dihydro-1H-benzimidazole-5-carboxamide trifluloroacetate

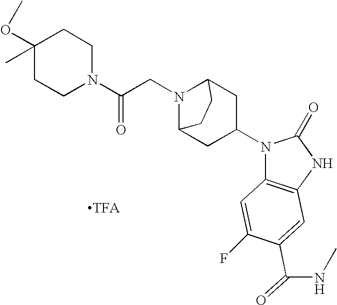

To a solution of 1-(bromoacetyl)-4-methoxy-4-methylpiperidine (0.63 g, 1.98 mmol) in DMF (15 mL), 1-(8-Aza-bicyclo[3.2.1]oct-3-yl)-6-fluoro-2-oxo-2,3-dihydro-1H-benzoimidazole-5-carboxylic acid methylamide (0.64 g, 2.57 mmol) was added followed by the addition of TEA (1 mL) at rt and maintained over night. The reaction mixture was diluted with DCM (250 mL), the organic layer was washed with water (250 mL), brine solution and concentrated to half of its volume. Then mixture was cooled to 0° C., TFA (5 mL) was added and maintained for 1 h. The resulted reaction mixture was concentrated to obtain the desired product as white solid (230 mg, 23%). IR (cm⁻¹): 3465, 3066, 2965, 1693, 1544, 1482, 1390, 1299 and 1189.

¹HNMR (DMSO): δ 11.25(s, 1H), 9.75 (s, 1H), 7.95-8.08 (m, 1H), 7.72 (d, 1H), 7.18-7.3 (m, 1H), 4.62-4.76 (m, 1H), 3.95-4.26 (m, 4H), 3.32-3.8 (m, 4H), 3.06-3.32 (m, 4H), 3.02 (t, 1H), 2.64-2.9 (m, 5H), 1.6-2.44 (m, 9H), 1.32-1.6 (m, 2H) and 1.12(s, 3H). LC-MS APCl (m/z)=488 (M+H)⁺.

### tert-butyl 4-(5-{[methoxy(methyl)amino]carbonyl}-2-oxo-2,3-dihydro-1H-benzimidazol-1-yl)piperidine-1-carboxylate

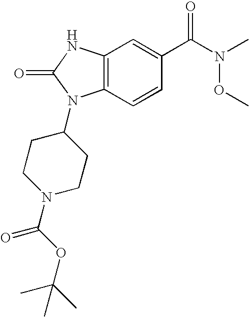

1-(1-tert-Butoxycarbonyl-piperidin-4-yl)-2-oxo-2,3-dihydro-1H-benzoimidazole-5-carboxylic acid (11.19 g, 31 mmol), dimethylhydroxylamine HCl (4.53 g, 46.4 mmol), PyBOP (19.3 g, 37.2 mmol) and diisopropylethylamine (16.2 ml, 93 mmol) were combined in dimethylformamide (100 ml). The reaction mixture was stirred at room temperature for 72 h under nitrogen atmosphere then diluted with dichloromethane (50 ml) and washed sequentially with 1N HCl, saturated sodium bicarbonate, and brine. The organic layer was dried over sodium sulfate, filtered and concentrated to a red syrup under reduced pressure. The residue was purified by a column chromatography with gradient 0-40% methanol in ethyl acetate (Biotage Horizon HPFC, HS-silica 40+M). The desired fractions were combined and concentrated to give a light orange powder (12.2 g, 97.1%). Experimental Data; LCMS (Phenomenex Develosil CombiRP3, 50×4.6 mm Column, 45° C., 50-2% water in 3.5 minutes, hold 0.5 minutes, run time=4 min.), Retention time 1.158 min; APCl⁺ 305.2 (M+1-Boc) and APl⁻ 403.3 (M−1).

### tert-butyl 4-(5-acetyl-2-oxo-2,3-dihydro-1H-benzimidazol-1-yl)piperidine-1-carboxylate

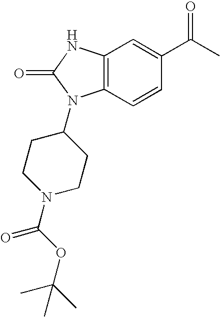

Dissolved was 4-[5-(Methoxy-methyl-carbamoyl)-2-oxo-2,3-dihydro-benzoimidazol-1-yl]-piperidine-1-carboxylic acid tert-butyl ester (6.0 g, 15 mmol) in anhydrous tetrahydrofuran (150 ml) and chilled to 0° C. on ice-salt bath under nitrogen atmosphere. Added was bromomethylmagnesium 1.4 M solution in toluene/tetrahydrofuran (53 ml, 74 mmol), dropwise, via pressure equalized addition funnel. The reaction mixture was stirred at 0° C. for 2.5 h., then warmed to room temperature. 10 ml aliquot of bromomethylmagnesium was added and continued stirring. The reaction was quenched by slowly adding saturated ammonium chloride solution (15 ml) while cooled on ice water bath. Heavy white precipitate formed. It was further concentrated under reduced pressure, diluted with water and ethyl acetate (100 ml/each). Solids were collected by a vacuum filtration. The filtrate was extracted aqueous phase with ethyl acetate (2×20 mL), the organic layers were combined, washed with brine, dried over sodium sulfate, filtered and concentrated to yield desired product as an off-white fine powder (3.5 g, 66%). Experimental Data; LCMS (Phenomenex Develosil CombiRP3, 50×4.6 mm Column, 45 deg C., 50-2% water in 3.5 minutes, hold 0.5 minutes, run time=4 min.), Retention time 1.42 min., C₁₉H₂₅N₃O₄, MW 359.4; Found APCl⁺ 260.2 (M+1-Boc) and APCl⁻ 358.4 (M−1).

### 5-acetyl-1-piperidin-4-yl-1,3-dihydro-2H-benzimidazol-2-one; TFA salt

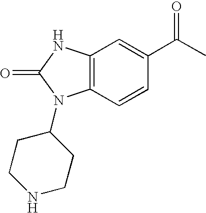

4-(5-Acetyl-2-oxo-2,3-dihydro-benzoimidazol-1-yl)-piperidine-1-carboxylic acid tert-butyl ester was dissolved in dichloromethane (10 ml), trifluoroacetic acid (10 ml) and stirred (ambient temperature) for 2 hrs. Reaction mixture was concentrated under reduced pressure, triturated/re-concentrated 4 times with dichloromethane to recover a light tan powder (4 g, 100%). Experimental Data; LCMS (Phenomenex Develosil CombiRP3, 50×4.6 mm Column, 45 deg C., 90-2% water in 3.5 minutes, hold 0.5 minutes, run time=4 min.), Retention time 1.09 min., C₁₄H₁₇N₃O₂, MW 259.1 parent; Found APCl⁺ 260.3 (M+1).

### EXAMPLE NO. 55

### 5-acetyl-1-{1-[2-(4-methoxy-4-methylpiperidin-1-yl)-2-oxoethyl]piperidin-4-yl}-1,3-dihydro-2H-benzimidazol-2-one; HCl salt

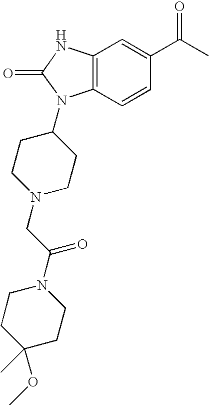

5-Acetyl-1-{1-[2-(4-methoxy-4-methyl-piperidin-1-yl)-2-oxo-ethyl]-piperidin-4-yl}-1,3-dihydro-benzoimidazol-2-one; TFA salt (470 mg, 1.2 mmol) was dissolved in 8 ml dry dimethylformamide, added triethylamine (1.0 ml, 7.2 mmol) and 2-Chloro-1-(4-methoxy-4-methyl-piperidin-1-yl)-ethanone (369 mg, 1.8 mmol) dissolved in 2 ml dimethylformamide. The reaction mixture was stirred at ambient temperature for 18 h under a nitrogen atmosphere. To the cloudy mixture added 5% sodium bicarbonate solution (9 ml), dropwise. Mixture became homogeneous, then precipitate formed on stirring. Added 10 ml water, mixture became homogeneous again. Extracted product mixture with ethyl acetate (30 ml) and dichloromethane (30 ml), combined organics, washed with brine, dried over sodium sulfate, filtered and concentrated under reduced pressure to yield an orange-brown glass. The resultant residue was purified using a column chromatography on silica using gradient 0-20% methanol in ethyl acetate. The desired fractions were combined and evaporated to yield a white powder. The material was dissolved in dichloromethane (5 ml) and methanol (1 ml), then added 2.0 M hydrogen chloride in diethyl ether (2.2 ml), stirred 45 minutes, and concentrated under reduced pressure. This gave a white HCl salt, which was dried in vacuum oven at 60° C. for 18 hours to recover a off-white powder (590 mg, 29%). Experimental Data; Micro Analysis; 1.0 C₂₃H₃₂N₄O₄/1.0 HCl/1.4H₂O; Calculated: C, 56.43%, H, 7.59%, N, 11.07%, Found: C, 56.35%, H, 7.36%, N, 11.43%. LCMS (Phenomenex Develosil CombiRP3, 50×4.6 mm Column, 45 deg C., 90-2% water in 3.5 minutes, hold 0.5 minutes, run time=4 min.), Retention time 2.16 min., C₂₃H₃₂N₄O₄, MW parent 428.5; Found APCl⁺ 429.3(M+1) and APCl⁻ 427.3 (M−1). Low resolution MS, 10V ionization, Found 429.2, 430.3, 431.3 (M+1) parent.

Serine Palmitoyl Transferase catalyses the first step in the sphingolipid biosynthetic pathway, the condensation of serine and Palmitoyl CoA producing 3-ketodihydrosphingosine and, subsequently, ceramides and sphingomyelin. This assay was developed to identify compounds inhibiting SPT activity since lowering sphingomyelin content of lipoproteins can increase lecithin cholesterol acyltransferase (LCAT) and lipoprotein lipase (LPL) activity which may lower triglycerides, raise HDL, and/or prevent or stabilize plaque formation. This assay utilizes microsomes derived from HEK 293 cells stably transfected with the long chain base 2 (LCB2) gene which codes for the human LCB2 subunit of SPT. The long chain base 1 (LCB1) subunit of SPT is expressed endogenously in this human cell line. The two subunits constitute SPT, however the LCB2 subunit is thought to impart the catalytic activity of SPT. Phospholipid coated flashplates are used to run reactions since the radiolabeled serine substrate is not bound by the phospholipid plate coating and, therefore, can be easily washed away whereas the labeled 3-ketodihydrosphigosine readily binds to the coated plates.

### SPT Flashplate Assay:

The SPT flashplate assay uses microsomes as an SPT enzyme source. These microsomes were treated with substrate (3H-serine and palmitoyl CoA), cofactors (pyridoxal 5-phosphate) and standard agents or putative drug candidates in a Perkin Elmer 96-well phospholipid coated flashplate (Flashplate Plus).

### Microsome Preparation:

HEK 293 LCB2 cells were grown in Dulbecco's Modified Eagle's Medium (DMEM) low glucose supplemented with 10% fetal bovine serum, 4-(2-hydroxyethyl)-1-piperazineethanesulfonic acid (HEPES) buffer (20 mM) and glutamine (2 mM). A cell paste of the HEK 293 LCB2 cells was homogenized in ice cold microsome buffer containing 125 mM sucrose, 3.0 mM imidazole and 5.0 mM dithiothreitol (DTT) pH 7.4. The protease inhibitors aprotinin, 0.2 mg/ml and leupeptin 0.2 mg/ml were also added. The cell paste was homogenized using 17 strokes of a power glass dounce homoginizer. The resultant slurry was centrifuged at 8000 rpm 4° C. for 20 minutes in a Beckman JA-20 rotor. The supernatant was then transferred, on ice, to new tubes and centrifuged for 60 minutes at 35,000 rpm and 4° C. in a Beckman Coulter Ti-70 rotor. The resulting pellet was then homogenized in ice cold microsome buffer using 17 strokes of a motorized glass Dounce homoginizer. The protein content was assayed using the Bio Rad protein assay (catalog no. 500-0006) per manufacturers instructions. Aliquots were stored in liquid nitrogen at a protein concentration between 10 and 20 mg/ml.

### SPT Assay:

Microsomes, test compound, standards, or controls, and isotope were combined in Perkin Elmer phospholipid coated flashplates as follows:

A microsome mix was prepared containing microsomes (14 ug/rx), DTT (4.5 mM final concentration), pyridoxal-L-phosphate (45 uM final concentration), HEPES/ethylenediaminetetraacetic acid (EDTA) buffer (90 mM HEPES, 2.3 mM EDTA final concentration) and dH20. Test compounds and myriocin standards were dissolved in DMSO and diluted in assay buffer (see above) to between 1.0 and 2.5%. The isotope labeling mix consisted of 3H serine (0.5 uCi/rx), unlabeled serine (3.5 uM final concentration), palmitoyl CoA (150 uM final concentration), Octylglucopyranoside (10 mM final concentration) and dH20. Using 96 well round bottom plates as reservoirs 35 uL microsome mix was added to flashplates followed by 5 uL compound, standard or control, and then 10 uL of the labeling mixture. All additions were made using a Beckman fx liquid handling robot. Plates were then sealed using foil plate sealers and incubated with shaking at room temperature 18-24 hours. Plates were then washed four times with Dulbeco's phosphate buffered saline using a 96 well plate washer, tapped free of excess moisture, allowed to air dry overnight, and read using a Tri-Lux beta counter in flashplate mode or using a Perkin Elmer Top Count.

### REFERENCES

1. Miyake Y. Kozutsumi Y. et al. Serine Palmitoyltransferase is the primary target of a sphingosine-like immunosuppressant, ISP-1/myriocin Biochemical and biophysical research communications. 1995; 211:396-403.
2. Jiang X. Paultre F. et al. Plasma sphingomyelin level as a risk factor for coronary artery disease. Arterioscler Thromb Vasc Biol. 2000; 20:2614-2618.
3. Hanada Kentaro H. Serine palmitoyltransferase, a key enzyme of sphingolipid metabolism. Biochimica et biophysica acta. 2003; 1632:16-30.

| Example No. | SPTISF IC₅₀ (nM) |
|---|---|
| 1 | 10 |
| 2 | 5.4 |
| 3 | 6.3 |
| 4 | 5.4 |
| 5 | 14 |
| 6 | 12 |
| 7 | 35 |
| 8 | 37 |
| 9 | 6.2 |
| 10 | 17 |
| 11 | 63 |
| 12 | 5.3 |
| 13 | 6.0 |
| 14 | 106 |
| 15 | 9.5 |
| 16 | 53 |
| 17 | 7.14 |
| 18 | (ND) |
| 19 | 18.7 |
| 20 | 11.4 |
| 21 | 14.6 |
| 22 | 10.6 |
| 23 | 8.4 |
| 24 | |
| 25 | 2.64 |
| 26 | 7.5 |
| 27 | 6 |
| 28 | 12.9 |
| 29 | 9.4 |
| 30 | 14.5 |
| 31 | 14.7 |
| 32 | 26 |
| 33 | 59.1 |
| 34 | 40-6 |
| 35 | 0.89 |
| 36 | 3.1 |
| 37 | 1.68 |
| 38 | 3.0 |
| 39 | 47.9 |
| 40 | 12.9 |
| 41 | 365 |
| 42 | 59 |
| 43 | 301 |
| 44 | 51.7 |
| 45 | 333 |
| 46 | 90-5 |
| 47 | 508 |
| 48 | 4,400 |
| 49 | 165 |
| 51 | 118 |
| 52 | 118 |
| 53 | 259 |
| 55 | 71 |

Additional compounds within the descriptions herein are:
1-{1-[2-(4-methoxyphenyl)-2-oxoethyl]piperidin-4-yl}-1,3-dihydro-2H-benzimidazol-2-one;
1-{1-[2-(4-bromophenyl)-2-oxoethyl]piperidin-4-yl}-1,3-dihydro-2H-benzimidazol-2-one;
1-{1-[2-(4-chlorophenyl)-2-oxoethyl]piperidin-4-yl}-1,3-dihydro-2H-indol-2-one;
methyl 1-{1-[2-(4-chlorophenyl)-2-oxoethyl]piperidin-4-yl}-2-oxo-2,3-dihydro-1H-benzimidazole-5-carboxylate;
5-chloro-1-{1-[2-(4-chlorophenyl)-2-oxoethyl]piperidin-4-yl}-1,3-dihydro-2H-benzimidazol-2-one;
5-chloro-1-{1-[2-(4-chlorophenyl)-2-oxoethyl]piperidin-4-yl}-1,3-dihydro-2H-benzimidazol-2-one;
1-{1-[2-(4-chlorophenyl)-2-oxoethyl]piperidin-4-yl}-5-fluoro-1,3-dihydro-2H-benzimidazol-2-one;
1-{1-[2-(4-chlorophenyl)-2-oxoethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-N-methyl-2-oxo-2,3-dihydro-1H-benzimidazole-5-carboxamide;
5-bromo-1-{1-[2-(4-chlorophenyl)-2-oxoethyl]piperidin-4-yl}-1,3-dihydro-2H-benzimidazol-2-one;
1-{1-[2-(4-chlorophenyl)-2-oxoethyl]piperidin-4-yl}-6-fluoro-N-methyl-2-oxo-2,3-dihydro-1H-benzimidazole-5-carboxamide;
methyl 1-{1-[2-(4-chlorophenyl)-2-oxoethyl]piperidin-4-yl}-6-fluoro-2-oxo-2,3-dihydro-1H-benzimidazole-5-carboxylate;
methyl 1-{1-[2-(4-chlorophenyl)-2-hydroxyethyl]piperidin-4-yl}-6-fluoro-2-oxo-2,3-dihydro-1H-benzimidazole-5-carboxylate;
1-{1-[2-(4-chlorophenyl)-2-hydroxyethyl]piperidin-4-yl}-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-2,3-dihydro-1H-benzimidazole-5-carboxylic acid;
1-{1-[2-(4-chlorophenyl)-2-oxoethyl]piperidin-4-yl}-2-oxo-2,3-dihydro-1H-benzimidazole-5-carbonitrile;
1-{1-[2-(4-chlorophenyl)-2-oxoethyl]piperidin-4-yl}-5-pyridin-2-yl-1,3-dihydro-2H-benzimidazol-2-one;
1-{1-[2-(4-chlorophenyl)-2-oxoethyl]piperidin-4-yl}-N-isopropyl-2-oxo-2,3-dihydro-1H-benzimidazole-5-carboxamide;
1-{1-[2-(4-chlorophenyl)-2-oxoethyl]piperidin-4-yl}-N-isopropyl-2-oxo-2,3-dihydro-1H-benzimidazole-5-carboxamide;
1-{1-[2-(4-chlorophenyl)-2-fluoroethyl]piperidin-4-yl}-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-N-methyl-2-oxo-2,3-dihydro-1H-benzimidazole-5-carboxamide;
1-{1-[2-(4-chlorophenyl)-2-hydroxyethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-N-(2-hydroxyethyl)-2-oxo-2,3-dihydro-1H-benzimidazole-5-carboxamide;
1-{1-[2-(4-chlorophenyl)-2-hydroxyethyl]piperidin-4-yl}-N-(2-hydroxyethyl)-2-oxo-2,3-dihydro-1H-benzimidazole-5-carboxamide;
1-{1-[2-(4-chlorophenyl)-2-oxoethyl]piperidin-4-yl}-5-(1H-tetrazol-5-yl)-1,3-dihydro-2H-benzimidazol-2-one;
1-{1-[2-(4-chlorophenyl)-2-hydroxyethyl]piperidin-4-yl}-5-(1H-tetrazol-5-yl)-1,3-dihydro-2H-benzimidazol-2-one;
1-{1-[2-(4-chlorophenyl)-2-hydroxyethyl]piperidin-4-yl}-5-(1H-tetrazol-5-yl)-1,3-dihydro-2H-benzimidazol-2-one;
1-{1-[2-(4-methoxyphenyl)-2-oxoethyl]piperidin-4-yl}-N-methyl-2-oxo-2,3-dihydro-1H-benzimidazole-5-carboxamide;
1-{1-[2-hydroxy-2-(4-methoxyphenyl)ethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-5-{5-oxo-4,5-dihydro-1,2,4-oxadiazol-3-yl}-1,3-dihydro-2H-benzimidazol-2-one;
N-methyl-1-{1-[2-(4-methylpiperidin-1-yl)-2-oxoethyl]piperidin-4-yl}-2-oxo-2,3-dihydro-1H-benzimidazole-5-carboxamide;
1-{1-[2-(4-chlorophenyl)-2-hydroxyethyl]piperidin-4-yl}-5-(5-oxo-4,5-dihydro-1,2,4-oxadiazol-3-yl)-1,3-dihydro-2H-benzimidazol-2-one;
1-{1-[2-(4-methylpiperidin-1-yl)-2-oxoethyl]piperidin-4-yl}-5-(5-oxo-4,5-dihydro-1,2,4-oxadiazol-3-yl)-1,3-dihydro-2H-benzimidazol-2-one;
N-methyl-2-oxo-1-(1-{2-oxo-2-[4-(trifluoromethyl)piperidin-1-yl]ethyl}piperidin-4-yl)-2,3-dihydro-1H-benzimidazole-5-carboxamide;
1-{1-[2-(4-chlorophenyl)-2-hydroxyethyl]piperidin-4-yl}-6-fluoro-N-(2-hydroxyethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-(2-methoxyethyl)-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-N-propyl-2,3-dihydro-1H-benzimidazole-5-carboxamide;
1-{1-[2-(4-chlorophenyl)-2-oxoethyl]piperidin-4-yl}-N-ethyl-2-oxo-2,3-dihydro-1H-benzimidazole-5-carboxamide;
1-{1-[2-(4-chlorophenyl)-2-oxoethyl]piperidin-4-yl}-N-ethyl-2-oxo-2,3-dihydro-1H-benzimidazole-5-carboxamide;
1-{1-[2-(4-chlorophenyl)-2-hydroxyethyl]piperidin-4-yl}-N-ethyl-2-oxo-2,3-dihydro-1H-benzimidazole-5-carboxamide;
1-{1-[2-(4-chlorophenyl)-2-hydroxyethyl]piperidin-4-yl}-N-ethyl-2-oxo-2,3-dihydro-1H-benzimidazole-5-carboxamide;
1-{1-[2-(4-isopropylpiperidin-1-yl)-2-oxoethyl]piperidin-4-yl}-N-methyl-2-oxo-2,3-dihydro-1H-benzimidazole-5-carboxamide;
methyl 3-{1-[2-(4-chlorophenyl)-2-oxoethyl]piperidin-4-yl}-2-oxo-2,3-dihydro-1,3-benzoxazole-6-carboxylate;
1-{1-[2-(1,3-dihydro-2H-isoindol-2-yl}-2-oxoethyl]piperidin-4-yl-N-methyl-2-oxo-2,3-dihydro-1H-benzimidazole-5-carboxamide;
1-{1-[2-(4-ethylpiperidin-1-yl)-2-oxoethyl]piperidin-4-yl}-N-methyl-2-oxo-2,3-dihydro-1H-benzimidazole-5-carboxamide;
1-{1-[2-(4-ethyl-4-methylpiperidin-1-yl)-2-oxoethyl]piperidin-4-yl}-N-methyl-2-oxo-2,3-dihydro-1H-benzimidazole-5-carboxamide; benzyl N-[(1-{1-[2-(4-chlorophenyl)-2-oxoethyl]piperidin-4-yl}-2-oxo-2,3-dihydro-1H-benzimidazol-5-yl)carbonyl]-beta-alaninate;
1-{1-[2-(4,4-dimethylpiperidin-1-yl)-2-oxoethyl]piperidin-4-yl}-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-N-methyl-2-oxo-2,3-dihydro-1H-benzimidazole-5-carboxamide;
N-methyl-2-oxo-1-{1-[2-oxo-2-(4-propylpiperidin-1-yl)ethyl]piperidin-4-yl}-2,3-dihydro-1H-benzimidazole-5-carboxamide;
N-methyl-2-oxo-1-{1-[2-oxo-2-(4-propylpiperidin-1-yl)ethyl]piperidin-4-yl}-2,3-dihydro-1H-benzimidazole-5-carboxamide;
1-{1-[2-(3-azaspiro[5.5]undec-3-yl)-2-oxoethyl]piperidin-4-yl}-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-N-{2-[(2-hydroxyethyl)thio]ethyl}-2-oxo-2,3-dihydro-1H-benzimidazole-5-carboxamide;
1-{1-[2-(4-chlorophenyl)-2-hydroxyethyl]piperidin-4-yl}-6-fluoro-N-(2-furylmethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-[2-(methylthio)ethyl]-2-oxo-2,3-dihydro-1H-benzimidazole-5-carboxamide;
1-{-[2-(4-chlorophenyl)-2-hydroxyethyl]piperidin-4-yl}-6-fluoro-5-{[4-(2-hydroxyethyl)piperazin-1-yl]carbonyl}-1,3-dihydro-2H-benzimidazol-2-one;
1-{1-[2-(4-chlorophenyl)-2-hydroxyethyl]piperidin-4-yl}-N-[3-(dimethylamino)propyl]-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-N-[2-(1H-imidazol-4-yl)ethyl]-2-oxo-2,3-dihydro-1H-benzimidazole-5-carboxamide;
1-{1-[2-(4-chlorophenyl)-2-hydroxyethyl]piperidin-4-yl}-6-fluoro-N-(2-isopropoxyethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-[(5-methylpyrazin-2-yl)methyl]-2-oxo-2,3-dihydro-1H-benzimidazole-5-carboxamide;
N-(sec-butyl)-1-{1-[2-(4-chlorophenyl)-2-hydroxyethyl]piperidin-4-yl}-6-fluoro-2-oxo-2,3-dihydro-1H-benzimidazole-5-carboxamide;
1-{1-[2-(4-chlorophenyl)-2-hydroxyethyl]piperidin-4-yl}-6-fluoro-N-(2-hydroxy-1-methylethyl)-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-N-[2-(2-oxoimidazolidin-1-yl)ethyl]-2,3-dihydro-1H-benzimidazole-5-carboxamide;
1-{1-[2-(4-chlorophenyl)-2-hydroxyethyl]piperidin-4-yl}-N-[(1-ethyl-1H-pyrazol-4-yl)methyl]-6-fluoro-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-N-(2-pyridin-4-ylethyl)-2,3-dihydro-1H-benzimidazole-5-carboxamide;
1-{1-[2-(4-chlorophenyl)-2-hydroxyethyl]piperidin-4-yl}-6-fluoro-2-oxo-N-[3-(2-oxopyrrolidin-1-yl)propyl]-2,3-dihydro-1H-benzimidazole-5-carboxamide;
1-(1-[2-(4-chlorophenyl)-2-hydroxyethyl]piperidin-4-yl)-6-fluoro-2-oxo-N-(2-propoxyethyl)-2,3-dihydro-1H-benzimidazole-5-carboxamide;
N-[4-(acetylamino)benzyl]-1-{1-[2-(4-chlorophenyl)-2-hydroxyethyl]piperidin-4-yl}-6-fluoro-2-oxo-2,3-dihydro-1H-benzimidazole-5-carboxamide;
N-methyl-2-oxo-1-{1-[2-oxo-2-(4-phenylpiperidin-1-yl)ethyl]piperidin-4-yl}-2,3-dihydro-1H-benzimidazole-5-carboxamide;
3-{1-[2-(4-chlorophenyl)-2-oxoethyl]piperidin-4-yl}-N-methyl-2-oxo-2,3-dihydro-1,3-benzoxazole-6-carboxamide;
3-{1-[2-(4-chlorophenyl)-2-oxoethyl]piperidin-4-yl}-N-ethyl-2-oxo-2,3-dihydro-1,3-benzoxazole-6-carboxamide;
1-{1-[2-(8-azaspiro[4.5]dec-8-yl)-2-oxoethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-N-methyl-2-oxo-2,3-dihydro-1H-benzimidazole-5-sulfonamide;
1-{1-[2-(4-chlorophenyl)-2-oxoethyl]piperidin-4-yl}-N-methyl-2-oxo-2,3-dihydro-1H-benzimidazole-5-sulfonamide;
1-{1-[2-(4-chlorophenyl)-2-oxoethyl]piperidin-4-yl}-5-(methylsulfonyl)-1,3-dihydro-2H-benzimidazol-2-one;
1-{1-[2-(4-chlorophenyl)-2-oxoethyl]piperidin-4-yl}-5-(methylsulfonyl)-1,3-dihydro-2H-benzimidazol-2-one;
1-{1-[2-(4,4-dimethylpiperidin-1-yl)-2-oxoethyl]piperidin-4-yl}-6-fluoro-N-methyl-2-oxo-2,3-dihydro-1H-benzimidazole-5-carboxamide;
1-{1-[2-(4-ethyl-4-methylpiperidin-1-yl)-2-oxoethyl]piperidin-4-yl}-6-fluoro-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-5-(5-oxo-4,5-dihydro-1,2,4-oxadiazol-3-yl)-1,3-dihydro-2H-benzimidazol-2-one;
1-{1-[2-(4-ethyl-4-methylpiperidin-1-yl)-2-oxoethyl]piperidin-4-yl}-5-(5-oxo-4,5-dihydro-1,2,4-oxadiazol-3-yl)-1,3-dihydro-2H-benzimidazol-2-one;
1-{1-[2-(4-chlorophenyl)-2-hydroxyethyl]piperidin-4-yl}-5-(methylsulfonyl)-1,3-dihydro-2H-benzimidazol-2-one;
1-{1-[2-(4-chlorophenyl)-2-hydroxyethyl]piperidin-4-yl}-N-methyl-2-oxo-2,3-dihydro-1H-benzimidazole-5-sulfonamide;
1-{1-[2-(4-chlorophenyl)-2-hydroxyethyl]piperidin-4-yl}-N-methyl-2-oxo-2,3-dihydro-1H-benzimidazole-5-sulfonamide;
1-{1-[2-(4,4-dimethylpiperidin-1-yl)-2-oxoethyl]piperidin-4-yl}-5-(methylsulfonyl)-1,3-dihydro-2H-benzimidazol-2-one;
1-{1-[2-(4,4-dimethylpiperidin-1-yl)-2-oxoethyl]piperidin-4-yl}-5-(methylsulfonyl)-1,3-dihydro-2H-benzimidazol-2-one;
1-{1-[2-(8-azaspiro[4.5]dec-8-yl}-2-oxoethyl]piperidin-4-yl-6-fluoro-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-N-ethyl-2-oxo-2,3-dihydro-1H-benzimidazole-5-carboxamide;
1-{1-[2-(4,4-dimethylpiperidin-1-yl)-2-oxoethyl]piperidin-4-yl}-N-ethyl-3-methyl-2-oxo-2,3-dihydro-1H-benzimidazole-5-carboxamide;
1-{1-[2-(4,4-dimethylpiperidin-1-yl)-2-oxoethyl]piperidin-4-yl}-N-methyl-2-oxo-2,3-dihydro-1H-benzimidazole-5-sulfonamide;
1-{1-[2-(4,4-dimethylpiperidin-1-yl)-2-oxoethyl]piperidin-4-yl}-N-methyl-2-oxo-2,3-dihydro-1H-benzimidazole-5-sulfonamide;
6-fluoro-N-methyl-1-{1-[2-(1-oxa-8-azaspiro[4.5]dec-8-yl)-2-oxoethyl]piperidin-4-yl}-2-oxo-2,3-dihydro-1H-benzimidazole-5-carboxamide;
6-fluoro-1-{1-[2-(4-methoxy-4-methylpiperidin-1-yl)-2-oxoethyl]piperidin-4-yl}-N-methyl-2-oxo-2,3-dihydro-1H-benzimidazole-5-carboxamide;
1-{1-[2-(4-ethyl-4-methoxypiperidin-1-yl)-2-oxoethyl]piperidin-4-yl}-6-fluoro-N-methyl-2-oxo-2,3-dihydro-1H-benzimidazole-5-carboxamide;
1-{1-[2-(3-azabicyclo[3.2.2]non-3-yl)-2-oxoethyl]piperidin-4-yl}-6-fluoro-N-methyl-2-oxo-2,3-dihydro-1H-benzimidazole-5-carboxamide;
1-{1-[2-(3-azabicyclo[3.2.1]oct-3-yl)-2-oxoethyl]piperidin-4-yl}-6-fluoro-N-methyl-2-oxo-2,3-dihydro-1H-benzimidazole-5-carboxamide;
6-fluoro-N-methyl-2-oxo-1-{1-[2-oxo-2-(1,8,8-trimethyl-3-azabicyclo[3.2.1]oct-3-yl)ethyl]piperidin-4-yl}-2,3-dihydro-1H-benzimidazole-5-carboxamide;
1-{1-[2-(4-ethylphenyl)-2-oxoethyl]piperidin-4-yl}-6-fluoro-N-methyl-2-oxo-2,3-dihydro-1H-benzimidazole-5-carboxamide;
1-{1-[2-(4-ethoxy-4-ethylpiperidin-1-yl)-2-oxoethyl]piperidin-4-yl}-6-fluoro-N-methyl-2-oxo-2,3-dihydro-1H-benzimidazole-5-carboxamide;
6-fluoro-N-methyl-1-{1-[2-(4-methylenepiperidin-1-yl)-2-oxoethyl]piperidin-4-yl}-2-oxo-2,3-dihydro-1H-benzimidazole-5-carboxamide;
1-{1-[2-(6-azaspiro[2.5]oct-6-yl)-2-oxoethyl]piperidin-4-yl}-6-fluoro-N-methyl-2-oxo-2,3-dihydro-1H-benzimidazole-5-carboxamide;
6-fluoro-1-(1-{2-[4-methoxy-4-(trifluoromethyl)piperidin-1-yl]-2-oxoethyl}piperidin-4-yl)-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-2-oxo-2,3-dihydro-1H-benzimidazole-5-carboxamide;
1-{1-[2-(4,4-dimethylpiperidin-1-yl)-2-oxoethyl]piperidin-4-yl}-2-oxo-2,3-dihydro-1H-benzimidazole-5-carbonitrile;
6-fluoro-1-{1-[2-(4-isopropyl-4-methoxypiperidin-1-yl)-2-oxoethyl]piperidin-4-yl}-N-methyl-2-oxo-2,3-dihydro-1H-benzimidazole-5-carboxamide;
5-acetyl-1-{1-[2-(4,4-dimethylpiperidin-1-yl)-2-oxoethyl]piperidin-4-yl}-6-fluoro-1,3-dihydro-2H-benzimidazol-2-one;
1-{1-[2-(4,4-dimethylpiperidin-1-yl)-2-oxoethyl]piperidin-4-yl}-N′-hydroxy-2-oxo-2,3-dihydro-1H-benzimidazole-5-carboximidamide;
1-{1-[2-(4,4-dimethylpiperidin-1-yl)-2-oxoethyl]piperidin-4-yl}-2-oxo-2,3-dihydro-1H-benzimidazole-5-carbothioamide;
1-{1-[2-(4,4-dimethylpiperidin-1-yl)-2-oxoethyl]piperidin-4-yl}-6-fluoro-5-(methylsulfonyl)-1,3-dihydro-2H-benzimidazol-2-one;
1-{1-[2-(4,4-dimethylpiperidin-1-yl)-2-oxoethyl]piperidin-4-yl}-6-fluoro-5-(methylsulfonyl)-1,3-dihydro-2H-benzimidazol-2-one;
6-fluoro-1-{1-[2-(hexahydrocyclopenta[c]pyrrol-2(1H)-yl)-2-oxoethyl]piperidin-4-yl}-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-N-(2-methoxyethyl)-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-N-(2-methoxyethyl)-2-oxo-2,3-dihydro-1H-benzimidazole-5-carboxamide;
ethyl 3-(1-{1-[2-(4,4-dimethylpiperidin-1-yl)-2-oxoethyl]piperidin-4-yl}-2-oxo-2,3-dihydro-1H-benzimidazol-5-yl)-1,2,4-oxadiazole-5-carboxylate;
6-fluoro-1-{1-[2-(4-methoxy-4-methylpiperidin-1-yl)-2-oxoethyl]piperidin-4-yl}-5-(methylsulfonyl)-1,3-dihydro-2H-benzimidazol-2-one;
6-fluoro-1-{1-[2-(4-methoxy-4-methylpiperidin-1-yl)-2-oxoethyl]piperidin-4-yl}-5-(methylsulfonyl)-1,3-dihydro-2H-benzimidazol-2-one;
N′-(acetyloxy)-1-{1-[2-(4,4-dimethylpiperidin-1-yl)-2-oxoethyl]piperidin-4-yl}-2-oxo-2,3-dihydro-1H-benzimidazole-5-carboximidamide;
1-{1-[2-(4,4-dimethylpiperidin-1-yl)-2-oxoethyl]piperidin-4-yl}-2-oxo-2,3-dihydro-1H-benzimidazole-5-carboxamide;
1-{1-[2-(4-ethylphenyl)-2-hydroxyethyl]piperidin-4-yl}-6-fluoro-N-methyl-2-oxo-2,3-dihydro-1H-benzimidazole-5-carboxamide;
5-acetyl-1-{1-[2-(4,4-dimethylpiperidin-1-yl)-2-oxoethyl]piperidin-4-yl}-6-fluoro-1,3-dihydro-2H-indol-2-one;
1-{1-[2-(4,4-dimethylpiperidin-1-yl)-2-oxoethyl]piperidin-4-yl}-6-fluoro-5-(1-hydroxyethyl)-1,3-dihydro-2H-indol-2-one;
1-{1-[2-(4,4-dimethylpiperidin-1-yl)-2-oxoethyl]piperidin-4-yl}-2-oxo-N′-(pyruvoyloxy)-2,3-dihydro-1H-benzimidazole-5-carboximidamide;
1-(1-{2-[(3aR,6aS)-hexahydrocyclopenta[c]pyrrol-2(1H)-yl]-2-oxoethyl}piperidin-4-yl)-2-oxo-2,3-dihydro-1H-benzimidazole-5-carboxamide;
6-fluoro-1-(1-{2-[(3aR,6aS)-hexahydrocyclopenta[c]pyrrol-2(1H)-yl]-2-oxoethyl}piperidin-4-yl)-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-N-(2-hydroxyethyl)-2-oxo-2,3-dihydro-1H-benzimidazole-5-carboxamide;
1-{1-[2-(4,4-dimethylpiperidin-1-yl)-2-oxoethyl]piperidin-4-yl}-N-methyl-2-oxoindoline-5-carboxamide;
1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide;
6-fluoro-1-(1-{2-[(3aR,6aS)-hexahydrocyclopenta[c]pyrrol-2(1H)-yl]-2-oxoethyl}piperidin-4-yl)-5-(methylsulfonyl)-1,3-dihydro-2H-benzimidazol-2-one;
1-{1-[2-(4,4-dimethylpiperidin-1-yl)-2-oxoethyl]piperidin-4-yl}-6-fluoro-N-methyl-2-oxoindoline-5-carboxamide;
6-fluoro-1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide;
5-acetyl-6-fluoro-1-(1-{2-[(3aR,6aS)-hexahydrocyclopenta[c]pyrrol-2(1H)-yl]-2-oxoethyl}piperidin-4-yl)-1,3-dihydro-2H-benzimidazol-2-one;
1-{1-[2-(4,4-dimethylpiperidin-1-yl)-2-oxoethyl]piperidin-4-yl}-6-methyl-5-(methylsulfonyl)-1,3-dihydro-2H-benzimidazol-2-one;
1-{1-[2-(4,4-dimethylpiperidin-1-yl)-2-oxoethyl]piperidin-4-yl}-5-(5-methyl-1,2,4-oxadiazol-3-yl)-1,3-dihydro-2H-benzimidazol-2-one;
1-{1-[2-(4,4-dimethylpiperidin-1-yl)-2-oxoethyl]piperidin-4-yl}-5-(5-methyl-1,2,4-oxadiazol-3-yl)-1,3-dihydro-2H-benzimidazol-2-one;
1-{1-[2-(4-chlorophenyl)-2-oxoethyl]piperidin-4-yl}-5-(5-methyl-1,2,4-oxadiazol-3-yl)-1,3-dihydro-2H-benzimidazol-2-one;
5-acetyl-1-(1-{2-[(3aR,6aS)-hexahydrocyclopenta[c]pyrrol-2(1H)-yl]-2-oxoethyl}piperidin-4-yl)-1,3-dihydro-2H-benzimidazol-2-one;
1-[1-(2-cyclohexyl-2-oxoethyl)piperidin-4-yl]-6-fluoro-N-methyl-2-oxo-2,3-dihydro-1H-benzimidazole-5-carboxamide;
1-[1-(2-cyclohexyl-2-oxoethyl)piperidin-4-yl]-6-fluoro-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-5-(5-oxo-4,5-dihydro-1,2,4-oxadiazol-3-yl)-1,3-dihydro-2H-benzimidazol-2-one;
N-methyl-1-{1-[2-(5-methylenehexahydrocyclopenta[c]pyrrol-2(1H)-yl)-2-oxoethyl]piperidin-4-yl}-2-oxo-2,3-dihydro-1H-benzimidazole-5-carboxamide;
1-(1-{2-[(3aR,6aS)-hexahydrocyclopenta[c]pyrrol-2(1H)-yl]-2-oxoethyl}piperidin-4-yl)-5-(5-methyl-1,2,4-oxadiazol-3-yl)-1,3-dihydro-2H-benzimidazol-2-one;
1-(1-{2-[(3aR,6aS)-hexahydrocyclopenta[c]pyrrol-2(1H)-yl]-2-oxoethyl}piperidin-4-yl)-5-[5-(trichloromethyl)-1,2,4-oxadiazol-3-yl]-1,3-dihydro-2H-benzimidazol-2-one;
1-{1-[2-(4-methoxy-4-methylpiperidin-1-yl)-2-oxoethyl]piperidin-4-yl}-6-methyl-5-(methylsulfonyl)-1,3-dihydro-2H-benzimidazol-2-one;
1-{1-[2-(4-methoxy-4-methylpiperidin-1-yl)-2-oxoethyl]piperidin-4-yl}-N-methyl-2-oxo-2,3-dihydro-1H-benzimidazole-5-carboxamide;
5-acetyl-1-{1-[2-(4-methoxy-4-methylpiperidin-1-yl)-2-oxoethyl]piperidin-4-yl}-1,3-dihydro-2H-benzimidazol-2-one;
5-acetyl-6-fluoro-1-{1-[2-(4-methoxy-4-methylpiperidin-1-yl)-2-oxoethyl]piperidin-4-yl}-1,3-dihydro-2H-benzimidazol-2-one;
1-{(1R,5S)-8-[2-(4,4-dimethylpiperidin-1-yl)-2-oxoethyl]-8-azabicyclo[3.2.1]oct-3-yl}-6-fluoro-N-methyl-2-oxo-2,3-dihydro-1H-benzimidazole-5-carboxamide;
5-acetyl-1-{1-[2-(6-azaspiro[2.5]oct-6-yl)-2-oxoethyl]piperidin-4-yl}-1,3-dihydro-2H-benzimidazol-2-one;
5-acetyl-1-{1-[2-(6-azaspiro[2.5]oct-6-yl)-2-oxoethyl]piperidin-4-yl}-6-fluoro-1,3-dihydro-2H-benzimidazol-2-one;
1-{1-[2-(4,4-dimethylpiperidin-1-yl)-2-oxoethyl]piperidin-4-yl}-5-(1,2,4-oxadiazol-3-yl)-1,3-dihydro-2H-benzimidazol-2-one;
6-fluoro-1-{(1R,5S)-8-[2-(hexahydrocyclopenta[c]pyrrol-2(1H)-yl)-2-oxoethyl]-8-azabicyclo[3.2.1]oct-3-yl}-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-5-(ethylsulfonyl)-1,3-dihydro-2H-benzimidazol-2-one;
5-isobutyryl-1-{1-[2-(4-methoxy-4-methylpiperidin-1-yl)-2-oxoethyl]piperidin-4-yl}-1,3-dihydro-2H-benzimidazol-2-one;
1-{1-[2-(4-chlorophenyl)-2-hydroxyethyl]piperidin-4-yl}-5-(5-methyl-1,2,4-oxadiazol-3-yl)-1,3-dihydro-2H-benzimidazol-2-one;
1-{1-[2-(6-azaspiro[2.5]oct-6-yl)-2-oxoethyl]piperidin-4-yl}-5-isobutyryl-1,3-dihydro-2H-benzimidazol-2-one;
methyl 1-(1-{2-[(3aR,6aS)-hexahydrocyclopenta[c]pyrrol-2(1H)-yl]-2-oxoethyl}piperidin-4-yl)-2-oxoindoline-5-carboxylate;
1-{1-[2-(4-ethylcyclohexyl)-2-oxoethyl]piperidin-4-yl}-5-(methylsulfonyl)-1,3-dihydro-2H-benzimidazol-2-one;
6-fluoro-1-{1-[2-(4-isopropylcyclohexyl)-2-oxoethyl]piperidin-4-yl}-N-methyl-2-oxo-2,3-dihydro-1H-benzimidazole-5-carboxamide;
1-{1-[2-(4-methoxy-4-methylpiperidin-1-yl)-2-oxoethyl]piperidin-4-yl}-N-methyl-2-oxoindoline-5-carboxamide;
5-isobutyryl-1-{1-[2-(octahydro-2H-isoindol-2-yl)-2-oxoethyl]piperidin-4-yl}-1,3-dihydro-2H-benzimidazol-2-one;
1-{1-[2-(6-azaspiro[2.5]oct-6-yl)-2-oxoethyl]piperidin-4-yl}-5-(methylsulfonyl)-1,3-dihydro-2H-benzimidazol-2-one;
1-{1-[2-(6-azaspiro[2.5]oct-6-yl)-2-oxoethyl]piperidin-4-yl}-N-methyl-2-oxoindoline-5-carboxamide;
6-fluoro-N-methyl-1-{1-[2-(1-methylcyclohexyl)-2-oxoethyl]piperidin-4-yl}-2-oxo-2,3-dihydro-1H-benzimidazole-5-carboxamide;
6-fluoro-1-{1-[2-(4-methoxycyclohexyl)-2-oxoethyl]piperidin-4-yl}-N-methyl-2-oxo-2,3-dihydro-1H-benzimidazole-5-carboxamide;
1-{1-[2-(4-methylcyclohexyl)-2-oxoethyl]piperidin-4-yl}-5-(methylsulfonyl)-1,3-dihydro-2H-benzimidazol-2-one;
1-{1-[2-(4-isopropylcyclohexyl)-2-oxoethyl]piperidin-4-yl}-5-(methylsulfonyl)-1,3-dihydro-2H-benzimidazol-2-one;
6-fluoro-N-methyl-1-{1-[2-(4-methylcyclohexyl)-2-oxoethyl]piperidin-4-yl}-2-oxo-2,3-dihydro-1H-benzimidazole-5-carboxamide;
5-(methylsulfonyl)-1-(1-{2-oxo-2-[4-(trifluoromethyl)cyclohexyl]ethyl}piperidin-4-yl)-1,3-dihydro-2H-benzimidazol-2-one;
1-(1-[2-(4,4-difluorocyclohexyl)-2-oxoethyl]piperidin-4-yl-5-(methylsulfonyl)-1,3-dihydro-2H-benzimidazol-2-one;
6-fluoro-N-methyl-2-oxo-1-(1-{2-oxo-2-[4-(trifluoromethyl)cyclohexyl]ethyl}piperidin-4-yl)-2,3-dihydro-1H-benzimidazole-5-carboxamide;
1-{1-[2-(4,4-difluorocyclohexyl)-2-oxoethyl]piperidin-4-yl}-6-fluoro-N-methyl-2-oxo-2,3-dihydro-1H-benzimidazole-5-carboxamide;
1-{1-[2-(4,4-difluorocyclohexyl)-2-oxoethyl]piperidin-4-yl}-6-fluoro-N-methyl-2-oxo-2,3-dihydro-1H-benzimidazole-5-carboxamide;
1-[1-(2-cyclohex-3-en-1-yl-2-oxoethyl)piperidin-4-yl]-6-fluoro-N-methyl-2-oxo-2,3-dihydro-1H-benzimidazole-5-carboxamide;
1-[1-(2-cyclohex-3-en-1-yl-2-oxoethyl)piperidin-4-yl]-N-methyl-2-oxoindoline-5-carboxamide;
1-{1-[2-(6-azaspiro[2.5]oct-6-yl)-2-oxoethyl]piperidin-4-yl}-6-fluoro-5-(methylsulfonyl)-1,3-dihydro-2H-benzimidazol-2-one;
1-{1-[2-(6-azaspiro[2.5]oct-6-yl)-2-oxoethyl]piperidin-4-yl}-6-methyl-5-(methylsulfonyl)-1,3-dihydro-2H-benzimidazol-2-one;
1-{1-[2-(4-ethylcyclohexyl)-2-oxoethyl]piperidin-4-yl}-6-fluoro-N-methyl-2-oxo-2,3-dihydro-1H-benzimidazole-5-carboxamide;
6-fluoro-1-{1-[2-(4-methoxy-4-methylcyclohexyl)-2-oxoethyl]piperidin-4-yl}-N-methyl-2-oxo-2,3-dihydro-1H-benzimidazole-5-carboxamide;
1-[1-(2-cyclohexyl-2-hydroxyethyl)piperidin-4-yl]-6-fluoro-N-methyl-2-oxo-2,3-dihydro-1H-benzimidazole-5-carboxamide;
6-fluoro-N-methyl-1-{1-[2-(octahydro-2H-isoindol-2-yl)-2-oxoethyl]piperidin-4-yl}-2-oxoindoline-5-carboxamide;
methyl 1-{1-[2-(octahydro-2H-isoindol-2-yl)-2-oxoethyl]piperidin-4-yl}-2-oxoindoline-5-carboxylate;
1-[1-(2-cyclohexyl-2-oxoethyl)piperidin-4-yl]-N-methyl-2-oxoindoline-5-carboxamide;
N-methyl-1-{1-[2-(octahydro-2H-isoindol-2-yl)-2-oxoethyl]piperidin-4-yl}-2-oxoindoline-5-carboxamide;
1-[1-(2-cyclohexyl-2-hydroxyethyl)piperidin-4-yl]-N-methyl-2-oxoindoline-5-carboxamide;
methyl 1-{1-[2-(6-azaspiro[2.5]oct-6-yl)-2-oxoethyl]piperidin-4-yl}-2-oxoindoline-5-carboxylate;
1-{1-[2-(4-methoxy-4-methylpiperidin-1-yl)-2-oxoethyl]piperidin-4-yl}-5-propionyl-1,3-dihydro-2H-benzimidazol-2-one;
6-fluoro-N-methyl-1-{1-[2-(trans-4-methylcyclohexyl)-2-oxoethyl]piperidin-4-yl}-2-oxo-2,3-dihydro-1H-benzimidazole-5-carboxamide;
1-{1-[2-(4-ethylcyclohexyl)-2-hydroxyethyl]piperidin-4-yl}-5-(methylsulfonyl)-1,3-dihydro-2H-benzimidazol-2-one;
1-{1-[2-(6-azaspiro[2.5]oct-6-yl)-2-oxoethyl]piperidin-4-yl}-6-fluoro-N-methyl-2-oxoindoline-5-carboxamide;
methyl 1-{1-[2-(4-methoxy-4-methylpiperidin-1-yl)-2-oxoethyl]piperidin-4-yl}-2-oxoindoline-5-carboxylate;
1-[1-(2-cyclopentyl-2-oxoethyl)piperidin-4-yl]-6-fluoro-N-methyl-2-oxo-2,3-dihydro-1H-benzimidazole-5-carboxamide;
5-acetyl-1-{1-[2-(octahydro-2H-isoindol-2-yl)-2-oxoethyl]piperidin-4-yl}-1,3-dihydro-2H-benzimidazol-2-one;
6-fluoro-1-{1-[2-(4-methoxy-4-methylpiperidin-1-yl)-2-oxoethyl]piperidin-4-yl}-N-methyl-2-oxoindoline-5-carboxamide;
1-(1-{2-[(3aR,6aS)-hexahydrocyclopenta[c]pyrrol-2(1H)-yl]-2-oxoethyl}piperidin-4-yl)-2-oxoindoline-5-carboxylic acid;
1-[1-(2-cycloheptyl-2-oxoethyl)piperidin-4-yl]-6-fluoro-N-methyl-2-oxo-2,3-dihydro-1H-benzimidazole-5-carboxamide;
1-[1-(2-cyclohexyl-2-oxoethyl)piperidin-4-yl]-5-(1,2,4-oxadiazol-3-yl)-1,3-dihydro-2H-benzimidazol-2-one;
5-(5-methyl-1,2,4-oxadiazol-3-yl)-1-{1-[2-(4-methylpiperidin-1-yl)-2-oxoethyl]piperidin-4-yl}-1,3-dihydro-2H-benzimidazol-2-one;
5-(methylsulfonyl)-1-{1-[2-(octahydro-2H-isoindol-2-yl)-2-oxoethyl]piperidin-4-yl}-1,3-dihydro-2H-benzimidazol-2-one;
1-{1-[2-(6-azaspiro[2.5]oct-6-yl)-2-oxoethyl]piperidin-4-yl}-5-propionyl-1,3-dihydro-2H-benzimidazol-2-one;
1-{-[2-(4,4-difluorocyclohexyl)-2-hydroxyethyl]piperidin-4-yl}-6-fluoro-N-methyl-2-oxo-2,3-dihydro-1H-benzimidazole-5-carboxamide;
1-{1-[2-(3,3-dimethylcyclobutyl)-2-oxoethyl]piperidin-4-yl}-N-methyl-2-oxoindoline-5-carboxamide;
6-fluoro-5-(methylsulfonyl)-1-{1-[2-(octahydro-2H-isoindol-2-yl)-2-oxoethyl]piperidin-4-yl}-1,3-dihydro-2H-benzimidazol-2-one;
1-{1-[2-(4-ethylcyclohexyl)-2-hydroxyethyl]piperidin-4-yl}-6-fluoro-N-methyl-2-oxo-2,3-dihydro-1H-benzimidazole-5-carboxamide;
1-[1-(2-bicyclo[3.1H]hex-6-yl-2-oxoethyl)piperidin-4-yl]-6-fluoro-N-methyl-2-oxo-2,3-dihydro-1H-benzimidazole-5-carboxamide;
1-[1-(2-cyclopentyl-2-hydroxyethyl)piperidin-4-yl]-6-fluoro-N-methyl-2-oxo-2,3-dihydro-1H-benzimidazole-5-carboxamide;
1-[1-(3-cyclohexyl-2-oxopropyl)piperidin-4-yl]-6-fluoro-N-methyl-2-oxo-2,3-dihydro-1H-benzimidazole-5-carboxamide;
6-fluoro-1-{1-[2-hydroxy-2-(1,2,3,4-tetrahydronaphthalen-2-yl)ethyl]piperidin-4-yl}-N-methyl-2-oxo-2,3-dihydro-1H-benzimidazole-5-carboxamide;
6-fluoro-N-methyl-2-oxo-1-{1-[2-oxo-2-(1,2,3,4-tetrahydronaphthalen-2-yl)ethyl]piperidin-4-yl}-2,3-dihydro-1H-benzimidazole-5-carboxamide;
1-[1-(2-cycloheptyl-2-hydroxyethyl)piperidin-4-yl]-6-fluoro-N-methyl-2-oxo-2,3-dihydro-1H-benzimidazole-5-carboxamide;
6-fluoro-1-{1-[2-(4-methoxy-4-methylcyclohexyl)-2-oxoethyl]piperidin-4-yl}-N-methyl-2-oxo-2,3-dihydro-1H-benzimidazole-5-carboxamide;
6-fluoro-N-methyl-1-{1-[2-(octahydro-2H-isoindol-2-yl)-2-oxoethyl]piperidin-4-yl}-2-oxo-2,3-dihydro-1H-benzimidazole-5-carboxamide;
ethyl 1-{1-[2-(6-azaspiro[2.5]oct-6-yl)-2-oxoethyl]piperidin-4-yl}-2-oxo-2,3-dihydro-1H-benzimidazole-5-carboxylate;
1-{1-[2-(6-azaspiro[2.5]oct-6-yl)-2-oxoethyl]piperidin-4-yl}-2-oxo-2,3-dihydro-1H-benzimidazole-5-carboxylic acid;
1-{1-[2-(6-azaspiro[2.5]oct-6-yl)-2-oxoethyl]piperidin-4-yl}-6-fluoro-2-oxo-2,3-dihydro-1H-benzimidazole-5-carboxylic acid;
ethyl 2-oxo-1-[1-(2-oxo-2-spiro[2.5]oct-6-ylethyl)piperidin-4-yl]indoline-5-carboxylate;
ethyl 1-{1-[2-(6-azaspiro[2.5]oct-6-yl)-2-oxoethyl]piperidin-4-yl}-6-fluoro-2-oxo-2,3-dihydro-1H-benzimidazole-5-carboxylate;
6-fluoro-1-{1-[2-hydroxy-2-(4-methylcyclohexyl)ethyl]piperidin-4-yl}-N-methyl-2-oxo-2,3-dihydro-1H-benzimidazole-5-carboxamide;
methyl 1-(1-[2-(4,4-difluorocyclohexyl)-2-oxoethyl]piperidin-4-yl)-6-fluoro-2-oxoindoline-5-carboxylate;
6-fluoro-1-{1-[2-(cis-4-methoxy-4-methylcyclohexyl)-2-oxoethyl]piperidin-4-yl}-N-methyl-2-oxo-2,3-dihydro-1H-benzimidazole-5-carboxamide;
methyl 6-fluoro-1-(1-{2-[(3aR,6aS)-hexahydrocyclopenta[c]pyrrol-2(1H)-yl]-2-oxoethyl}piperidin-4-yl)-2-oxo-2,3-dihydro-1H-benzimidazole-5-carboxylate;
methyl 6-fluoro-1-(1-{2-[(3aR,6aS)-hexahydrocyclopenta[c]pyrrol-2(1H)-yl]-2-oxoethyl}piperidin-4-yl)-2-oxoindoline-5-carboxylate;
ethyl 6-fluoro-1-{1-[2-(4-methoxy-4-methylpiperidin-1-yl)-2-oxoethyl]piperidin-4-yl}-2-oxo-2,3-dihydro-1H-benzimidazole-5-carboxylate; and
ethyl 1-{1-[2-(4-methoxy-4-methylpiperidin-1-yl)-2-oxoethyl]piperidin-4-yl}-2-oxo-2,3-dihydro-1H-benzimidazole-5-carboxylate;
   or a pharmaceutically acceptable salt, hydrate or solvate thereof.

What is claimed:

1. A compound of the formula: 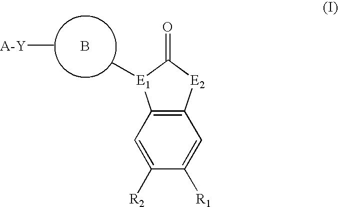
wherein:
E₁ is selected from N or CH;
E₂ is selected from NR, O or CRₐR_{b};
R is H, C₁-C₃ alkyl, —CH₂—COOH, —CH₂—COO—C₁-C₆ alkyl;
Rₐ and R_{b} are independently selected from H or C₁-C₃ alkyl;
Y is selected from the linking groups: 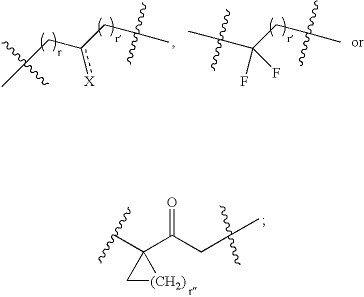
the dashed line [symbol] connected to X indicates an optional double bond;
r is an integer of from 0 to 2;
r′ is an integer from 1 to 3;
r″ is an integer from 1 to 3;
X is H, halogen, OH, oxo or ═NOR′; wherein R′ is hydrogen, C₁-C₆ straight or branched chain alkyl, C₃-C₆ cycloalkyl or —C₁-C₃ alkyl)-C₃-C₆ cycloalkyl;
The B ring represents a moiety selected from the group of: 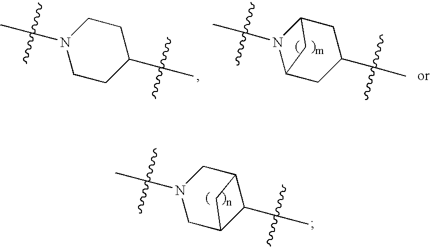
m and n are each independently 0 to 2;
A is a C₁-C₆ alkyl, C₂-C₆ alkenyl, a carbocycle, or a heterocycle; each of the alkyl, alkenyl, carbocycle and heterocycle groups being optionally substituted by R₃ and R₄;
R₁ is selected from:
a) H, halogen, CN, —C(O)R⁵, —C(O)OR⁵, —C(O)NR⁵R⁶, —S(O)ₚR⁵, S(O)₂N R⁵R⁶ and C₁-C₃ alkyl optionally substituted by OH; or
b) a moiety of the formula: 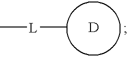
L is a linking group selected from a bond, a C₁-C₃ alkylene group, —C(O)—, —C(O)NR⁵—, —C(O)O—, —S(O)ₚ— and —S(O)₂NR⁵—;
p is 0 to 2;
D represents a moiety selected from —(CH₂)₀₋₃-carbocycle and —(CH₂)₀₋₃-heterocycle; each optionally substituted by from 1 to 3 groups independently selected from OH, oxo, CN, NH₂, NO₂, CF₃, halogen, C₁-C₃ alkyl, —O—C₁-C₃ alkyl, —S—C₁-C₃ alkyl, —NHC(O)R⁵, —O—C₁-C₃ alkyl, —S—C₁-C₃ alkyl, —(CH₂)₀₋₃—C(O)R⁵, —(CH₂)₀₋₃—C(O)OR⁵, —(CH₂)₀₋₃—C(O)NR⁵R⁶, —S(O)ₚR⁵, and S(O)₂NR⁵R⁶; wherein the C₁-C₃ alkyl, —O—C₁-C₃ alkyl, and —S—C₁-C₃ alkyl groups may be substituted by OH;
R⁵ is selected from H, C₁-C₆ alkyl, and —(CH₂)₀₋₃—(C₃-C₇ cycloalkyl); wherein the C₁-C₆ alkyl, and —(CH₂)₀₋₃—(C₃-C₇ cycloalkyl) groups may be optionally substituted by from 1 or 3 groups selected from OH, —O—C₁-C₃ alkyl, —S—C₁-C₃ alkyl, —COOR⁶, —NH₂, —NH(C₁-C₆ alkyl), —N(C₁-C₆ alkyl)₂, halogen, CF₃, CN, —NC(O)R⁶, and —OC(O)R⁶;
R⁶ is selected from H, C₁-C₆ alkyl, C₁-C₆ alkenyl, —(CH₂)₀₋₃-carbocycle and —(CH₂)₀₋₃-heterocycle;
R₂ is selected from the group of H, halogen, CF₃, C₁-C₃ alkoxy, C₁-C₃ alkyl and C₂-C₃ alkenyl; the C₁-C₃ alkoxy, C₁-C₃ alkyl and C₂-C₃ alkenyl groups being optionally substituted by from 1 to 3 halogen, OH, C₁-C₃ alkoxy or CN groups;
R₃ and R₄ are independently selected from H, halogen, CN, OH, oxo, C₁-C₆ alkyl, C₃-C₆ cycloalkyl, —CH₂—C₃-C₆ cycloalkyl, C₁-C₃ haloalkyl, —O—C₁-C₆ alkyl, —S—C₁-C₆ alkyl, phenyl, benzyl, NR_{y}R_{z}, —C(O)NR_{y}R_{z}, COOR_{y}, —C₁-C₃ alkyl-OH, —S(O)ᵣ—R_{y};
R_{y} and R_{z} are independently selected from H, C₁-C₆ alkyl, C₃-C₆ cycloalkyl, —CH₂—C₃-C₆ cycloalkyl, phenyl, benzyl,
r′″ is an integer selected from 1 or 2;
or R₃ and R₄ together form a fused or spiro 3-, 4-, 5- or 6-membered carbocyclic or heterocyclic ring;
or a pharmaceutically acceptable salt form thereof.

2. A compound of claim 1 of the formula: 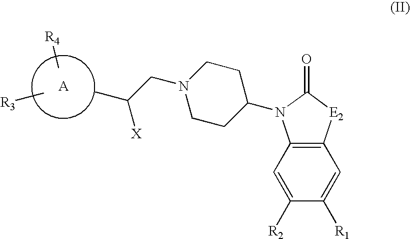
wherein:
X is H, halo, OH or oxo;
E₂ is selected from NR, CRₐR_{b} and O;
Rₐ and R_{b} are independently selected from H or C₁-C₃ alkyl;
A is a C₁-C₆ alkyl, C₂-C₆ alkenyl, C₃-C₈ cycloalkyl, phenyl, piperidine, piperazine, morpholine, octahydro-cyclopentapyrrole or aza-bicyclo-nonane group;
R₁ is selected from:
a) H, halogen, CN, —C(O)R⁵, —C(O)OR⁵, —C(O)NR⁵R⁶, —S(O)ₚR⁵S(O)₂N R⁵R⁶ and C₁-C₃ alkyl optionally substituted by OH; or
b) a moiety of the formula: 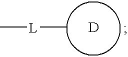
L is a linking group selected from a bond, a C₁-C₃ alkylene group, —C(O)—, —C(O)NR⁵—, —C(O)O—, —S(O)ₚ— and —S(O)₂NR⁵—;
p is 0 to 2;
D represents a moiety selected from —(CH₂)₀₋₃-carbocycle, and —(CH₂)₀₋₃-heterocycle; with the cycloalkyl, phenyl, heterocycle and heteroaryl groups on these moieties being optionally substituted by from 1 to 3 groups independently selected from OH, oxo, CN, NH₂, NO₂, CF₃, halogen, C₁-C₃ alkyl, —O—C₁-C₃ alkyl, —S—C₁-C₃ alkyl, —NHC(O)R⁵, —O—C₁-C₃ alkyl, —S—C₁-C₃ alkyl, —(CH₂)₀₋₃—C(O)R⁵, —(CH₂)₀₋₃—C(O)OR⁵, —(CH₂)₀₋₃—C(O)NR⁵R⁶, —S(O)ₚR⁵, and S(O)ₚNR⁵R⁶; wherein the C₁-C₃ alkyl, —O—C₁-C₃ alkyl, —S—C₁-C₃ alkyl may be substituted by OH;
R₂ is selected from the group of H, halo, CF₃, C₁-C₃ alkoxy or C₁-C₃ alkyl; the alkyl, alkoxy and alkenyl groups being optionally substituted by from 1 to 3 halogen, OH, C₁-C₃ alkoxy or CN groups;
R₃ and R₄ are independently selected from H, halo, CN, OH, oxo, C₁-C₆ alkyl, C₃-C₆ cycloalkyl, —CH₂—C₃-C₆ cycloalkyl, C₁-C₃ haloalkyl, —O—C₁-C₆ alkyl, —S—C₁-C₆ alkyl, phenyl, benzyl, NR_{y}R_{z}, —C(O)NR_{y}R_{z}, COOR_{y}, —C₁-C₃ alkyl-OH, —S(O)_{r′″}—R_{y};
R_{y} and R_{z} are independently selected from H, C₁-C₆ alkyl, C₃-C₆ cycloalkyl, —CH₂—C₃-C₆ cycloalkyl, phenyl, benzyl,
r′″ is an integer selected from 1 or 2;
or R₃ and R₄ together form a fused or Spiro 3-, 4-, 5- or 6-membered carbocyclic or heterocyclic ring;
or a pharmaceutically acceptable salt, hydrate or solvate thereof.

3. A compound of claim 1 of the formula: 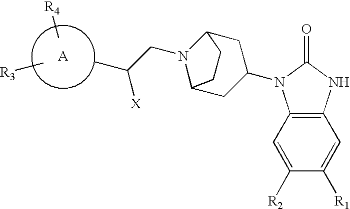
Wherein X, R₁, R₂, R₃, R₄ and A are as defined in claim 2; or a pharmaceutically acceptable salt, hydrate or solvate form thereof.

4. A compound of claim 1 selected from the group of:
1-{1-[2-(4-Chloro-phenyl)-2-oxo-ethyl]-piperidin-4-yl}-2-oxo-2,3-di-hydro-1H-benzoimidazole-5-carboxylic acid methyl amide;
1-{1-[2-(4-Chloro-phenyl)-2-oxo-ethyl]-piperidin-4-yl}-2-oxo-2,3-dihydro-1H-benzoimidazole-5-carboxylic acid methylamide;
1-{1-[2-(4-chloro-phenyl)-2-oxo-ethyl]-piperidin-4-yl}-6-fluoro-2-oxo-2,3-dihydro-1H-benzoimidazole-5-carboxylic acid methyl amide;
1-{1-[2-(4-chloro-phenyl)-2-hydroxy-ethyl]-piperidin-4-yl}-6-fluoro-2-oxo-2,3-dihydro-1H-benzoimidazole-5-carboxylic acid methyl amide;
1-{1-[2-(4-chloro-phenyl)-2-oxo-ethyl]-piperidin-4-yl}-6-fluoro-2-oxo-2,3-dihydro-1H-benzoimidazole-5-carboxylic acid methyl ester;
1-{1-[2-(4-chloro-phenyl)-2-hydroxy-ethyl]-piperidin-4-yl}-6-fluoro-2-oxo-2,3-dihydro-1H-benzoimidazole-5-carboxylic acid methyl ester;
1-{1-[2-(4-chloro-phenyl)-2-hydroxy-ethyl]-piperidin-4-yl}-6-fluoro-2-oxo-2,3-dihydro-1H-benzoimidazole-5-carboxylic acid;
1-{1-[2-(4-chloro-phenyl)-2-fluoro-ethyl]-piperidin-4-yl}-6-fluoro-2-oxo-2,3-dihydro-1H-benzoimidazole-5-carboxylic acid methyl amide;
1-[2-(4-ethyl-phenyl)-2-oxo-ethyl]-piperidin-4-yl)-6-fluoro-2-oxo-2,3-dihydro-1H-benzoimidazole-5-carboxylic acid methyl amide;
1-{-[2-(4-ethyl-phenyl)-2-hydroxy-ethyl]-piperidin-4-yl}-6-fluoro-2-oxo-2,3-dihydro-1H-benzoimidazole-5-carboxylic acid methyl amide;
1-[1-(2-Cyclohexyl-2-oxo-ethyl)-piperidin-4-yl]-6-fluoro-2-oxo-2,3-dihydro-1H-benzoimidazole-5-carboxylic acid methyl amide;
6-Fluoro-1-{1-[2-(4-methyl-cyclohexyl)-2-oxo-ethyl]-piperidin-4-yl}-2-oxo-2,3-dihydro-1H-benzoimidazole-5-carboxylic acid methylamide;
6-Fluoro-1-{1-[2-(4-methyl-cyclohexyl)-2-oxo-ethyl]-piperidin-4-yl}-2-oxo-2,3-dihydro-1H-benzoimidazole-5-carboxylic acid methylamide;
1-{1-[2-(4-tert-Butyl-cyclohexyl)-2-oxo-ethyl]-piperidin-4-yl}-6-fluoro-2-oxo-2,3-dihydro-1H-benzoimidazole-5-carboxylic acid methylamide;
6-Fluoro-2-oxo-1-{1-[2-oxo-2-(4-trifluoromethyl-cyclohexyl)-ethyl]-piperidin-4-yl}-2,3-dihydro-1H-benzoimidazole-5-carboxylicacid methylamide;
6-Fluoro-1-{1-[2-(4-methoxy-4-methyl-cyclohexyl)-2-oxo-ethyl]-piperidin-4-yl}-2-oxo-2,3-dihydro-1H-benzoimidazole-5-carboxylic acid methylamide;
6-Fluoro-1-{1-[2-(1-methyl-cyclohexyl)-2-oxo-ethyl]-piperidin-4-yl}-2-oxo-2,3-dihydro-1H-benzoimidazole-5-carboxylic acid methylamide;
1-{1-[2-(4-Methoxy-4-methyl-piperidin-1-yl)-2-oxo-ethyl]-piperidin-4-yl}-2-oxo-2,3-dihydro-1H-indole-5-carboxylic acid methyl amide;
1-{1-[2-(4-Methoxy-4-methyl-piperidin-1-yl)-2-oxo-ethyl]-piperidin-4-yl}-2-oxo-2,3-dihydro-1H-indole-5-carboxylic acid methylamide;
1-{1-[2-(hexahydro-cyclopenta[c]pyrrol-2-yl)-2-oxo-ethyl]-piperidin-4-yl}-2-oxo-2,3-dihydro-1H-indole-5-carboxylic acid methylamide;
6-fluoro-1-{1-[2-(hexahydro-cyclopenta[c]pyrrol-2-yl)-2-oxo-ethyl]-piperidin-4-yl}-2-oxo-2,3-dihydro-1H-indole-5-carboxylic acid methylamide;
1-{1-[2-(4,4-dimethyl-piperidin-1-yl)-2-oxo-ethyl]-piperidin-4-yl}-6-fluoro-2-oxo-2,3-dihydro-1H-indole-5-carboxylic acid methylamide;
1-{1-[2-(4,4-dimethyl-piperidin-1-yl)-2-oxo-ethyl]-piperidin-4-yl}-2-oxo-2,3-dihydro-1H-indole-5-carboxylic acid methylamide;
1-{1-[2-(6-Aza-spiro[2.5]oct-6-yl)-2-oxo-ethyl]-piperidin-4-yl}-2-oxo-2,3-dihydro-1H-indole-5-carboxylic acid methyl amide;
1-{1-[2-(6-Aza-spiro[2.5]oct-6-yl)-2-oxo-ethyl]-piperidin-4-yl}-2-oxo-2,3-dihydro-1H-indole-5-carboxylic acid methyl amide;
1-{1-[2-(4,4-dimethyl-piperidin-1-yl)-2-oxo-ethyl]-piperidin-4-yl}-6-fluoro-2-oxo-2,3-dihydro-1H-benzoimidazole-5-carboxylic acid methylamide;
1-{1-[2-(4-ethyl-4-methyl-piperidin-1-yl)-2-oxo-ethyl]-piperidin-4-yl}-6-fluoro-2-oxo-2,3-dihydro-1H-benzoimidazole-5-carboxylic acid methylamide;
1-{1-[2-(6-aza-spiro[2.5]oct-6-yl)-2-oxo-ethyl]-piperidin-4-yl}-6-fluoro-2-oxo-2,3-dihydro-1H-benzoimidazole-5-carboxylic acid methylamide;
6-fluoro-1-{1-[2-(4-methylene-piperidin-1-yl)-2-oxo-ethyl]-piperidin-4-yl}-2-oxo-2,3-dihydro-1H-benzoimidazole-5-carboxylic acid methylamide;
1-{1-[2-(3-aza-bicyclo[3.2.2]non-3-yl)-2-oxo-ethyl]-piperidin-4-yl}-6-fluoro-2-oxo-2,3-dihydro-1H-benzoimidazole-5-carboxylic acid methylamide;
6-fluoro-1-{1-[2-(hexahydro-cyclopenta[c]pyrrol-2-yl)-2-oxo-ethyl]-piperidin-4-yl}-2-oxo-2,3-dihydro-1H-benzoimidazole-5-carboxylic acid methylamide;
6-fluoro-1-{1-[2-(4-methoxy-4-methyl-piperidin-1-yl)-2-oxo-ethyl]-piperidin-4-yl}-2-oxo-2,3-dihydro-1H-benzoimidazole-5-carboxylic acid methylamide;
1-{1-[2-(4,4-dimethyl-piperidin-1-yl)-2-oxo-ethyl]-piperidin-4-yl}-3-methyl-2-oxo-2,3-dihydro-1H-benzoimidazole-5-carboxylic acid ethylamide;
1-{1-[2-(4,4-dimethyl-piperidin-1-yl)-2-oxo-ethyl]-piperidin-4-yl}-2-oxo-2,3-dihydro-1H-benzoimidazole-5-carboxylic acid ethylamide;
1-{1-[2-(4-chloro-phenyl)-2-oxo-ethyl]-piperidin-4-yl}-5-(1H-tetrazol-5-yl-1,3-dihydro-benzoimidazol-2-one;
1-{1-[2-(4-chloro-phenyl)-2-hydroxy-ethyl]-piperidin-4-yl}-5-(1H-tetrazol-5-yl)-1,3-dihydro-benzoimidazol-2-one;
1-{1-[2-(4-chloro-phenyl)-2-oxo-ethyl]-piperidin-4-yl}-5-(5-oxo-4,5-dihydro-[1,2,4]oxadiazol-3-yl)-1,3-dihydro-benzoimidazol-2-one;
1-{1-[2-(4-chloro-phenyl)-2-hydroxy-ethyl]-piperidin-4-yl-5-(5-oxo-4,5-dihydro-[1,2,4]oxadiazol-3-yl}-1,3-dihydro-benzoimidazol-2-one;
1-{1-[2-(4,4-Dimethyl-piperidin-1-yl)-2-oxo-ethyl]-piperidin-4-yl}-5-methanesulfonyl-1,3-dihydro-benzoimidazol-2-one;
1-{1-[2-(4,4-Dimethyl-piperidin-1-yl)-2-oxo-ethyl]-piperidin-4-yl}-5-methanesulfonyl-1,3-dihydro-benzoimidazol-2-one hydrochloride;
1-[1-(2-Cyclohexyl-2-oxo-ethyl)-piperidin-4-yl]-5-methanesulfonyl-1,3-dihydro-benzoimidazol-2-one;
5-Methanesulfonyl-1-{1-[2-(4-methyl-cyclohexyl)-2-oxo-ethyl]-piperidin-4-yl}-1,3-dihydro-benzoimidazol-2-one;
5-Methanesulfonyl-1-{1-[2-(4-methoxy-cyclohexyl)-2-oxo-ethyl]-piperidin-4-yl}-1,3-dihydro-benzoimidazol-2-one;
5-Methanesulfonyl-1-{1-[2-oxo-2-(4-trifluoromethyl-cyclohexyl)-ethyl]-piperidin-4-yl}-1,3-dihydro-benzoimidazol-2-one;
1-{1-[2-(4-tert-Butyl-cyclohexyl)-2-oxo-ethyl]-piperidin-4-yl}-5-methanesulfonyl-1,3-dihydro-benzoimidazol-2-one;
1-{1-[2-(4-isopropyl-cyclohexyl)-2-oxo-ethyl]-piperidin-4-yl}-5-methanesulfonyl-1,3-dihydro-benzoimidazol-2-one;
5-Methanesulfonyl-1-{1-[2-(4-methoxy-4-methyl-cyclohexyl)-2-oxo-ethyl]-piperidin-4-yl}-1,3-dihydro-benzoimidazol-2-one;
5-Methanesulfonyl-1-{1-[2-(1-methyl-cyclohexyl)-2-oxo-ethyl]-piperidin-4-yl}-1,3-dihydro-benzoimidazol-2-one;
6-Fluoro-1-{8-[2-(4-methoxy-4-methyl-piperidin-1-yl)-2-oxo-ethyl]-8-aza-bicyclo[3.2.1]oct-3-yl}-2-oxo-2,3-dihydro-1H-benzoimidazole-5-carboxylic acid methylamide;
1-{8-[2-(4,4-Dimethyl-piperidin-1-yl)-2-oxo-ethyl]-8-aza-bicyclo[3.2.1]oct-3-yl}-5-methanesulfonyl-1,3-dihydro-benzoimidazol-2-one;
5-Methanesulfonyl-1-{8-[2-(4-methoxy-4-methyl-piperidin-1-yl)-2-oxo-ethyl]-8-aza-bicyclo[3.2.1]oct-3-yl}-1,3-dihydro-benzoimidazol-2-one;
1-{8-[2-(Hexahydro-cyclopenta[c]pyrrol-2-yl)-2-oxo-ethyl]-8-aza-bicyclo[3.2.1]oct-3-yl}-5-methanesulfonyl-1,3-dihydro-benzoimidazol-2-one;
6-Methanesulfonyl-3-{1-[2-(octahydro-pentalen-2-yl)-2-oxo-ethyl]-piperidin-4-yl}-1,3-dihydro-indol-2-one; and
5-Acetyl-1-{1-[2-(4-methoxy-4-methyl-piperidin-1-yl)-2-oxo-ethyl]-piperidin-4-yl}-1,3-dihydro-benzoimidazol-2-one;
1-{1-[3-(4-Chloro-phenyl)-2-oxo-propyl]-piperidin-4-yl}-6-fluoro-2-oxo-2,3-dihydro-1Hbenzoimidazole5-carboxylic acid methylamide;
1-(1-{2-[1-(4-Chloro-phenyl)-cyclopropyl]-2-oxo-ethyl}-piperidin-4-yl)-6-fluoro-2-oxo-2,3-dihydro-1H-benzoimidazole-5-carboxylic acid methylamide;
1-(1-{2-[1-(4-Chloro-phenyl)-cyclobutyl]-2-oxo-ethyl}-piperidin-4-yl)-6-fluoro-2-oxo-2,3-dihydro-1H-benzoimidazole-5-carboxylic acid methylamide;
1-{1-[2-(3,3-dimethylcyclobutyl)-2-oxoethyl]piperidin-4-yl}-N-methyl-2-oxoindoline-5-carboxamide;
trans-6-fluoro-1-{1-[2-(4-methoxy-4-methyl-cyclohexyl)-2-oxo-ethyl]-piperidin-4-yl}-2-oxo-2,3-dihydro-1H-benzoimidazole-5-carboxylic acid methylamide;
cis-6-fluoro-1-{1-[2-(4-methoxy-4-methyl-cyclohexyl)-2-oxo-ethyl]-piperidin-4-yl}-2-oxo-2,3-dihydro-1H-benzoimidazole-5-carboxylic acid methylamide
1-(2-Cycloheptyl-2-oxo-ethyl)-4-(6-fluoro-5-methylcarbamoyl-2-oxo-2,3-dihydro-benzoimidazol-1-yl)-piperidinium;
1-(2-Cyclopentyl-2-oxo-ethyl)-4-(6-fluoro-5-methylcarbamoyl-2-oxo-2,3-dihydro-benzoimidazol-1-yl)-piperidinium;
4-(6-Fluoro-5-methylcarbamoyl-2-oxo-2,3-dihydro-benzoimidazol-1-yl)-1-[2-(4-methyl-cyclohexyl)-2-oxo-ethyl]-piperidinium;
or a pharmaceutically acceptable salt, hydrate or solvate form thereof.

5. A compound of claim 1 selected from the group of:
1-{1-[2-(4-methoxyphenyl)-2-oxoethyl]piperidin-4-yl}-1,3-dihydro-2H-benzimidazol-2-one;
1-{1-[2-(4-bromophenyl)-2-oxoethyl]piperidin-4-yl}-1,3-dihydro-2H-benzimidazol-2-one;
1-{1-[2-(4-chlorophenyl)-2-oxoethyl]piperidin-4-yl}-1,3-dihydro-2H-indol-2-one;
methyl 1-{1-[2-(4-chlorophenyl)-2-oxoethyl]piperidin-4-yl}-2-oxo-2,3-dihydro-1H-benzimidazole-5-carboxylate;
5-chloro-1-{1-[2-(4-chlorophenyl)-2-oxoethyl]piperidin-4-yl}-1,3-dihydro-2H-benzimidazol-2-one;
5-chloro-1-{1-[2-(4-chlorophenyl)-2-oxoethyl]piperidin-4-yl}-1,3-dihydro-2H-benzimidazol-2-one;
1-{1-[2-(4-chlorophenyl)-2-oxoethyl]piperidin-4-yl}-5-fluoro-1,3-dihydro-2H-benzimidazol-2-one;
1-{1-[2-(4-chlorophenyl)-2-oxoethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-N-methyl-2-oxo-2,3-dihydro-1H-benzimidazole-5-carboxamide;
5-bromo-1-{1-[2-(4-chlorophenyl)-2-oxoethyl]piperidin-4-yl}-1,3-dihydro-2H-benzimidazol-2-one;
1-{1-[2-(4-chlorophenyl)-2-oxoethyl]piperidin-4-yl}-6-fluoro-N-methyl-2-oxo-2,3-dihydro-1H-benzimidazole-5-carboxamide;
methyl 1-{1-[2-(4-chlorophenyl)-2-oxoethyl]piperidin-4-yl}-6-fluoro-2-oxo-2,3-dihydro-1H-benzimidazole-5-carboxylate;
methyl 1-{1-[2-(4-chlorophenyl)-2-hydroxyethyl]piperidin-4-yl}-6-fluoro-2-oxo-2,3-dihydro-1H benzimidazole-5-carboxylate;
1-{1-[2-(4-chlorophenyl)-2-hydroxyethyl]piperidin-4-yl}-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-2,3-dihydro-1H-benzimidazole-5-carboxylic acid;
1-{1-[2-(4-chlorophenyl)-2-oxoethyl]piperidin-4-yl}-2-oxo-2,3-dihydro-1H-benzimidazole-5-carbonitrile;
1-{1-[2-(4-chlorophenyl)-2-oxoethyl]piperidin-4-yl}-5-pyridin-2-yl-1,3-dihydro-2H-benzimidazol-2-one;
1-{1-[2-(4-chlorophenyl)-2-oxoethyl]piperidin-4-yl}-N-isopropyl-2-oxo-2,3-dihydro-1H-benzimidazole-5-carboxamide;
1-{1-[2-(4-chlorophenyl)-2-oxoethyl]piperidin-4-yl}-N-isopropyl-2-oxo-2,3-dihydro-1H-benzimidazole-5-carboxamide;
1-{1-[2-(4-chlorophenyl)-2-fluoroethyl]piperidin-4-yl}-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-N-methyl-2-oxo-2,3-dihydro-1H-benzimidazole-5-carboxamide;
1-{1-[2-(4-chlorophenyl)-2-hydroxyethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-N-(2-hydroxyethyl)-2-oxo-2,3-dihydro-1H-benzimidazole-5-carboxamide;
1-{1-[2-(4-chlorophenyl)-2-hydroxyethyl]piperidin-4-yl}-N-(2-hydroxyethyl)-2-oxo-2,3-dihydro-1H-benzimidazole-5-carboxamide;
1-{1-[2-(4-chlorophenyl)-2-oxoethyl]piperidin-4-yl}-5-(1H-tetrazol-5-yl)-1,3-dihydro-2H-benzimidazol-2-one;
1-{1-[2-(4-chlorophenyl)-2-hydroxyethyl]piperidin-4-yl}-5-(1H-tetrazol-5-yl)-1,3-dihydro-2H-benzimidazol-2-one;
1-{1-[2-(4-chlorophenyl)-2-hydroxyethyl]piperidin-4-yl}-5-(1H-tetrazol-5-yl)-1,3-dihydro-2H-benzimidazol-2-one;
1-{1-[2-(4-methoxyphenyl)-2-oxoethyl]piperidin-4-yl}-N-methyl-2-oxo-2,3-dihydro-1H-benzimidazole-5-carboxamide;
1-{1-[2-hydroxy-2-(4-methoxyphenyl)ethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-5-(5-oxo-4,5-dihydro-1,2,4-oxadiazol-3-yl)-1,3-dihydro-2H-benzimidazol-2-one;
N-methyl-1-{1-[2-(4-methylpiperidin-1-yl)-2-oxoethyl]piperidin-4-yl}-2-oxo-2,3-dihydro-1H-benzimidazole-5-carboxamide;
1-{1-[2-(4-chlorophenyl)-2-hydroxyethyl]piperidin-4-yl}-5-(5-oxo-4,5-dihydro-1,2,4-oxadiazol-3-yl)-1,3-dihydro-2H-benzimidazol-2-one;
1-{1-[2-(4-methylpiperidin-1-yl)-2-oxoethyl]piperidin-4-yl}-5-(5-oxo-4,5-dihydro-1,2,4-oxadiazol-3-yl)-1,3-dihydro-2H-benzimidazol-2-one;
N-methyl-2-oxo-1-(1-{2-oxo-2-[4-(trifluoromethyl)piperidin-1-yl]ethyl}piperidin-4-yl)-2,3-dihydro-1H-benzimidazole-5-carboxamide;
1-{1-[2-(4-chlorophenyl)-2-hydroxyethyl]piperidin-4-yl}-6-fluoro-N-(2-hydroxyethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-(2-methoxyethyl)-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-N-propyl-2,3-dihydro-1H-benzimidazole-5-carboxamide;
1-{1-[2-(4-chlorophenyl)-2-oxoethyl]piperidin-4-yl}-N-ethyl-2-oxo-2,3-dihydro-1H-benzimidazole-5-carboxamide;
1-{1-[2-(4-chlorophenyl)-2-oxoethyl]piperidin-4-yl}-N-ethyl-2-oxo-2,3-dihydro-1H-benzimidazole-5-carboxamide;
1-{-[2-(4-chlorophenyl)-2-hydroxyethyl]piperidin-4-yl}-N-ethyl-2-oxo-2,3-dihydro-1H-benzimidazole-5-carboxamide;
1-{1-[2-(4-chlorophenyl)-2-hydroxyethyl]piperidin-4-yl}-N-ethyl-2-oxo-2,3-dihydro-1H-benzimidazole-5-carboxamide;
1-{1-[2-(4-isopropylpiperidin-1-yl)-2-oxoethyl]piperidin-4-yl}-N-methyl-2-oxo-2,3-dihydro-1H-benzimidazole-5-carboxamide;
methyl 3-{1-[2-(4-chlorophenyl)-2-oxoethyl]piperidin-4-yl}-2-oxo-2,3-dihydro-1,3-benzoxazole-6-carboxylate;
1-{1-[2-(1,3-dihydro-2H-isoindol-2-yl)-2-oxoethyl]piperidin-4-yl}-N-methyl-2-oxo-2,3-dihydro-1H-benzimidazole-5-carboxamide;
1-{1-[2-(4-ethylpiperidin-1-yl)-2-oxoethyl]piperidin-4-yl}-N-methyl-2-oxo-2,3-dihydro-1H-benzimidazole-5-carboxamide;
1-{1-[2-(4-ethyl-4-methylpiperidin-1-yl)-2-oxoethyl]piperidin-4-yl}-N-methyl-2-oxo-2,3-dihydro-1H-benzimidazole-5-carboxamide;
benzyl N-[(1-{1-[2-(4-chlorophenyl)-2-oxoethyl]piperidin-4-yl}-2-oxo-2,3-dihydro-1H-benzimidazol-5-yl)carbonyl]-beta-alaninate;
1-{1-[2-(4,4-dimethylpiperidin-1-yl)-2-oxoethyl]piperidin-4-yl}-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-N-methyl-2-oxo-2,3-dihydro-1H-benzimidazole-5-carboxamide;
N-methyl-2-oxo-1-{1-[2-oxo-2-(4-propylpiperidin-1-yl)ethyl]piperidin-4-yl}-2,3-dihydro-1H-benzimidazole-5-carboxamide;
N-methyl-2-oxo-1-{1-[2-oxo-2-(4-propylpiperidin-1-yl)ethyl]piperidin-4-yl}-2,3-dihydro-1H-benzimidazole-5-carboxamide;
1-{1-[2-(3-azaspiro[5.5]undec-3-yl}-2-oxoethyl]piperidin-4-yl-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-N-(2-[(2-hydroxyethyl)thio]ethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-(2-furylmethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-[2-(methylthio)ethyl]-2-oxo-2,3-dihydro-1H-benzimidazole-5-carboxamide;
1-{1-[2-(4-chlorophenyl)-2-hydroxyethyl]piperidin-4-yl}-6-fluoro-5-{[4-(2-hydroxyethyl)piperazin-1-yl]carbonyl}-1,3-dihydro-2H-benzimidazol-2-one;
1-{1-[2-(4-chlorophenyl)-2-hydroxyethyl]piperidin-4-yl}-N-[3-(dimethylamino)propyl]-6-fluoro-N-methyl-2-oxo-2,3-dihydro-1H-benzimidazole-5-carboxamide;
1-{1-[2-(4-chlorophenyl)-2-hydroxyethyl]piperidin-4-yl}-6-fluoro-N-[2-(1H-imidazol-4-yl)ethyl]-2-oxo-2,3-dihydro-1H-benzimidazole-5-carboxamide;
1-{-[2-(4-chlorophenyl)-2-hydroxyethyl]piperidin-4-yl}-6-fluoro-N-(2-isopropoxyethyl)-2-oxo-2,3-dihydro-1H-benzimidazole-5-carboxamide;
1-{1-[2-(4-chlorophenyl)-2-hydroxyethyl]piperidin-4-yl}-6-fluoro-N-[(5-methylpyrazin-2-yl)methyl]-2-oxo-2,3-dihydro-1H-benzimidazole-5-carboxamide;
N-(sec-butyl)-1-{1-[2-(4-chlorophenyl)-2-hydroxyethyl]piperidin-4-yl}-6-fluoro-2-oxo-2,3-dihydro-1H-benzimidazole-5-carboxamide;
1-{1-[2-(4-chlorophenyl)-2-hydroxyethyl]piperidin-4-yl}-6-fluoro-N-(2-hydroxy-1-methylethyl)-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-N-[2-(2-oxoimidazolidin-1-yl)ethyl]-2,3-dihydro-1H-benzimidazole-5-carboxamide;
1-{1-[2-(4-chlorophenyl)-2-hydroxyethyl]piperidin-4-yl}-N-[(1-ethyl-1H-pyrazol-4-yl)methyl]-6-fluoro-2-oxo-2,3-dihydro-1H-benzimidazole-5-carboxamide;
1-{1-[2-(4-chlorophenyl)-2-hydroxyethyl]piperidin-4-yl}-6-fluoro-2-oxo-N-(2-pyridin-4-ylethyl)-2,3-dihydro-1H-benzimidazole-5-carboxamide;
1-{1-[2-(4-chlorophenyl)-2-hydroxyethyl]piperidin-4-yl}-6-fluoro-2-oxo-N-[3-(2-oxopyrrolidin-1-yl)propyl]-2,3-dihydro-1H-benzimidazole-5-carboxamide;
1-{1-[2-(4-chlorophenyl)-2-hydroxyethyl]piperidin-4-yl}-6-fluoro-2-oxo-N-(2-propoxyethyl)-2,3-dihydro-1H-benzimidazole-5-carboxamide;
N-[4-(acetylamino)benzyl]-1-{1-[2-(4-chlorophenyl)-2-hydroxyethyl]piperidin-4-yl}-6-fluoro-2-oxo-2,3-dihydro-1H-benzimidazole-5-carboxamide;
N-methyl-2-oxo-1-{1-[2-oxo-2-(4-phenylpiperidin-1-yl)ethyl]piperidin-4-yl}-2,3-dihydro-1H-benzimidazole-5-carboxamide;
3-{1-[2-(4-chlorophenyl)-2-oxoethyl]piperidin-4-yl}-N-methyl-2-oxo-2,3-dihydro-1,3-benzoxazole-6-carboxamide;
3-{1-[2-(4-chlorophenyl)-2-oxoethyl]piperidin-4-yl}-N-ethyl-2-oxo-2,3-dihydro-1,3-benzoxazole-6-carboxamide;
1-{1-[2-(8-azaspiro[4.5]dec-8-yl)-2-oxoethyl]piperidin-4-yl}-N-methyl-2-oxo-2,3-dihydro-1H-benzimidazole-5-carboxamide;
1-{1-[2-(4-chlorophenyl)-2-oxoethyl]piperidin-4-yl}-N-methyl-2-oxo-2,3-dihydro-1H-benzimidazole-5-sulfonamide;
1-{1-[2-(4-chlorophenyl)-2-oxoethyl]piperidin-4-yl}-N-methyl-2-oxo-2,3-dihydro-1H-benzimidazole-5-sulfonamide;
1-{1-[2-(4-chlorophenyl)-2-oxoethyl]piperidin-4-yl}-5-(methylsulfonyl)-1,3-dihydro-2H-benzimidazol-2-one;
1-{1-[2-(4-chlorophenyl)-2-oxoethyl]piperidin-4-yl}-5-(methylsulfonyl)-1,3-dihydro-2H-benzimidazol-2-one;
1-{1-[2-(4,4-dimethylpiperidin-1-yl)-2-oxoethyl]piperidin-4-yl}-6-fluoro-N-methyl-2-oxo-2,3-dihydro-1H-benzimidazole-5-carboxamide;
1-{1-[2-(4-ethyl-4-methylpiperidin-1-yl)-2-oxoethyl]piperidin-4-yl}-6-fluoro-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-5-(5-oxo-4,5-dihydro-1,2,4-oxadiazol-3-yl)-1,3-dihydro-2H-benzimidazol-2-one;
1-{1-[2-(4-ethyl-4-methylpiperidin-1-yl)-2-oxoethyl]piperidin-4-yl}-5-(5-oxo-4,5-dihydro-1,2,4-oxadiazol-3-yl)-1,3-dihydro-2H-benzimidazol-2-one;
1-(1-[2-(4-chlorophenyl)-2-hydroxyethyl]piperidin-4-yl)-5-(methylsulfonyl)-1,3-dihydro-2H-benzimidazol-2-one;
1-{1-[2-(4-chlorophenyl)-2-hydroxyethyl]piperidin-4-yl}-N-methyl-2-oxo-2,3-dihydro-1H-benzimidazole-5-sulfonamide;
1-{1-[2-(4-chlorophenyl)-2-hydroxyethyl]piperidin-4-yl}-N-methyl-2-oxo-2,3-dihydro-1H-benzimidazole-5-sulfonamide;
1-{1-[2-(4,4-dimethylpiperidin-1-yl)-2-oxoethyl]piperidin-4-yl}-5-(methylsulfonyl)-1,3-dihydro-2H-benzimidazol-2-one;
1-{1-[2-(4,4-dimethylpiperidin-1-yl)-2-oxoethyl]piperidin-4-yl}-5-(methylsulfonyl)-1,3-dihydro-2H-benzimidazol-2-one;
1-{1-(1-[2-(8-azaspiro[4.5]dec-8-yl)-2-oxoethyl]piperidin-4-yl}-6-fluoro-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-N-ethyl-2-oxo-2,3-dihydro-1H-benzimidazole-5-carboxamide;
1-{1-[2-(4,4-dimethylpiperidin-1-yl)-2-oxoethyl]piperidin-4-yl}-N-ethyl-3-methyl-2-oxo-2,3-dihydro-1H-benzimidazole-5-carboxamide;
1-{1-[2-(4,4-dimethylpiperidin-1-yl)-2-oxoethyl]piperidin-4-yl}-N-methyl-2-oxo-2,3-dihydro-1H-benzimidazole-5-sulfonamide;
1-{1-[2-(4,4-dimethylpiperidin-1-yl)-2-oxoethyl]piperidin-4-yl}-N-methyl-2-oxo-2,3-dihydro-1H-benzimidazole-5-sulfonamide;
6-fluoro-N-methyl-1-{1-[2-(1-oxa-8-azaspiro[4.5]dec-8-yl}-2-oxoethyl]piperidin-4-yl-2-oxo-2,3-dihydro-1H-benzimidazole-5-carboxamide;
6-fluoro-1-{1-[2-(4-methoxy-4-methylpiperidin-1-yl}-2-oxoethyl]piperidin-4-yl-N-methyl-2-oxo-2,3-dihydro-1H-benzimidazole-5-carboxamide;
1-{1-[2-(4-ethyl-4-methoxypiperidin-1-yl)-2-oxoethyl]piperidin-4-yl}-6-fluoro-N-methyl-2-oxo-2,3-dihydro-1H-benzimidazole-5-carboxamide;
1-{1-[2-(3-azabicyclo[3.2.2]non-3-yl)-2-oxoethyl]piperidin-4-yl}-6-fluoro-N-methyl-2-oxo-2,3-dihydro-1H-benzimidazole-5-carboxamide;
1-{1-[2-(3-azabicyclo[3.2.1]oct-3-yl)-2-oxoethyl]piperidin-4-yl}-6-fluoro-N-methyl-2-oxo-2,3-dihydro-1H-benzimidazole-5-carboxamide;
6-fluoro-N-methyl-2-oxo-1-{1-[2-oxo-2-(1,8,8-trimethyl-3-azabicyclo[3.2.1]oct-3-yl)ethyl]piperidin-4-yl}-2,3-dihydro-1H-benzimidazole-5-carboxamide;
1-{1-[2-(4-ethylphenyl)-2-oxoethyl]piperidin-4-yl}-6-fluoro-N-methyl-2-oxo-2,3-dihydro-1H-benzimidazole-5-carboxamide;
1-{1-[2-(4-ethoxy-4-ethylpiperidin-1-yl)-2-oxoethyl]piperidin-4-yl}-6-fluoro-N-methyl-2-oxo-2,3-dihydro-1H-benzimidazole-5-carboxamide;
6-fluoro-N-methyl-1-{1-[2-(4-methylenepiperidin-1-yl)-2-oxoethyl]piperidin-4-yl}-2-oxo-2,3-dihydro-1H-benzimidazole-5-carboxamide;
1-{1-[2-(6-azaspiro[2.5]oct-6-yl)-2-oxoethyl]piperidin-4-yl}-6-fluoro-N-methyl-2-oxo-2,3-dihydro-1H-benzimidazole-5-carboxamide;
6-fluoro-1-(1-{2-[4-methoxy-4-(trifluoromethyl)piperidin-1-yl]-2-oxoethyl}piperidin-4-yl-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-2-oxo-2,3-dihydro-1H-benzimidazole-5-carboxamide;
1-{1-[2-(4,4-dimethylpiperidin-1-yl)-2-oxoethyl]piperidin-4-yl}-2-oxo-2,3-dihydro-1H-benzimidazole-5-carbonitrile;
6-fluoro-1-{1-[2-(4-isopropyl-4-methoxypiperidin-1-yl)-2-oxoethyl]piperidin-4-yl}-N-methyl-2-oxo-2,3-dihydro-1H-benzimidazole-5-carboxamide;
5-acetyl-1-{1-[2-(4,4-dimethylpiperidin-1-yl)-2-oxoethyl]piperidin-4-yl}-6-fluoro-1,3-dihydro-2H-benzimidazol-2-one;
1-{1-[2-(4,4-dimethylpiperidin-1-yl)-2-oxoethyl]piperidin-4-yl}-N′-hydroxy-2-oxo-2,3-dihydro-1H-benzimidazole-5-carboximidamide;
1-{1-[2-(4,4-dimethylpiperidin-1-yl)-2-oxoethyl]piperidin-4-yl}-2-oxo-2,3-dihydro-1H-benzimidazole-5-carbothioamide;
1-{1-[2-(4,4-dimethylpiperidin-1-yl)-2-oxoethyl]piperidin-4-yl}-6-fluoro-5-(methylsulfonyl)-1,3-dihydro-2H-benzimidazol-2-one;
1-{1-[2-(4,4-dimethylpiperidin-1-yl)-2-oxoethyl]piperidin-4-yl}-6-fluoro-5-(methylsulfonyl)-1,3-dihydro-2H-benzimidazol-2-one;
6-fluoro-1-{1-[2-(hexahydrocyclopenta[c]pyrrol-2(1H)-yl)-2-oxoethyl]piperidin-4-yl}-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-N-(2-methoxyethyl)-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-N-(2-methoxyethyl)-2-oxo-2,3-dihydro-1H-benzimidazole-5-carboxamide;
ethyl 3-(1-{1-[2-(4,4-dimethylpiperidin-1-yl)-2-oxoethyl]piperidin-4-yl}-2-oxo-2,3-dihydro-1H-benzimidazol-5-yl)-1,2,4-oxadiazole-5-carboxylate;
6-fluoro-1-{1-[2-(4-methoxy-4-methylpiperidin-1-yl)-2-oxoethyl]piperidin-4-yl}-5-(methylsulfonyl)-1,3-dihydro-2H-benzimidazol-2-one;
6-fluoro-1-{1-[2-(4-methoxy-4-methylpiperidin-1-yl)-2-oxoethyl]piperidin-4-yl}-5-(methylsulfonyl)-1,3-dihydro-2H-benzimidazol-2-one;
N′-(acetyloxy)-1-{1-[2-(4,4-dimethylpiperidin-1-yl)-2-oxoethyl]piperidin-4-yl}-2-oxo-2,3-dihydro-1H-benzimidazole-5-carboximidamide;
1-{1-[2-(4,4-dimethylpiperidin-1-yl)-2-oxoethyl]piperidin-4-yl}-2-oxo-2,3-dihydro-1H-benzimidazole-5-carboxamide;
1-{1-[2-(4-ethylphenyl)-2-hydroxyethyl]piperidin-4-yl}-6-fluoro-N-methyl-2-oxo-2,3-dihydro-1H-benzimidazole-5-carboxamide;
5-acetyl-1-{1-[2-(4,4-dimethylpiperidin-1-yl)-2-oxoethyl]piperidin-4-yl}-6-fluoro-1,3-dihydro-2H-indol-2-one;
1-{1-[2-(4,4-dimethylpiperidin-1-yl)-2-oxoethyl]piperidin-4-yl}-6-fluoro-5-(1-hydroxyethyl)-1,3-dihydro-2H-indol-2-one;
1-{1-[2-(4,4-dimethylpiperidin-1-yl)-2-oxoethyl]piperidin-4-yl}-2-oxo-N′-(pyruvoyloxy)-2,3-dihydro-1H-benzimidazole-5-carboximidamide;
1-(1-{2-[(3aR,6aS)-hexahydrocyclopenta[c]pyrrol-2(1H)-yl]-2-oxoethyl}piperidin-4-yl)-2-oxo-2,3-dihydro-1H-benzimidazole-5-carboxamide;
6-fluoro-1-(1-{2-[(3aR,6aS)-hexahydrocyclopenta[c]pyrrol-2(1H)-yl]-2-oxoethyl}piperidin-4-yl)-2-oxo-2,3-dihydro-1H-benzimidazole-5-carboxamide;
1-(1-[2-(4,4-dimethylpiperidin-1-yl)-2-oxoethyl]piperidin-4-yl-6-fluoro-N-(2-hydroxyethyl)-2-oxo-2,3-dihydro-1H-benzimidazole-5-carboxamide;
1-{1-[2-(4,4-dimethylpiperidin-1-yl)-2-oxoethyl]piperidin-4-yl}-N-methyl-2-oxoindoline-5-carboxamide;
1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide;
6-fluoro-1-(1-{2-[(3aR,6aS)-hexahydrocyclopenta[c]pyrrol-2(1H)-yl]-2-oxoethyl}piperidin-4-yl)-5-(methylsulfonyl)-1,3-dihydro-2H-benzimidazol-2-one;
1-{1-[2-(4,4-dimethylpiperidin-1-yl)-2-oxoethyl]piperidin-4-yl}-6-fluoro-N-methyl-2-oxoindoline-5-carboxamide;
6-fluoro-1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide;
5-acetyl-6-fluoro-1-(1-{2-[(3aR,6aS)-hexahydrocyclopenta[c]pyrrol-2(1H)-yl]-2-oxoethyl}piperidin-4-yl)-1,3-dihydro-2H-benzimidazol-2-one;
1-{1-[2-(4,4-dimethylpiperidin-1-yl)-2-oxoethyl]piperidin-4-yl}-6-methyl-5-(methylsulfonyl)-1,3-dihydro-2H-benzimidazol-2-one;
1-{1-[2-(4,4-dimethylpiperidin-1-yl)-2-oxoethyl]piperidin-4-yl}-5-(5-methyl-1,2,4-oxadiazol-3-yl)-1,3-dihydro-2H-benzimidazol-2-one;
1-{1-[2-(4,4-dimethylpiperidin-1-yl)-2-oxoethyl]piperidin-4-yl}-5-(5-methyl-1,2,4-oxadiazol-3-yl)-1,3-dihydro-2H-benzimidazol-2-one;
1-{1-[2-(4-chlorophenyl)-2-oxoethyl]piperidin-4-yl}-5-(5-methyl-1,2,4-oxadiazol-3-yl)-1,3-dihydro-2H-benzimidazol-2-one;
5-acetyl-1-(1-{2-[(3aR,6aS)-hexahydrocyclopenta[c]pyrrol-2(1H)-yl]-2-oxoethyl}piperidin-4-yl)-1,3-dihydro-2H-benzimidazol-2-one;
1-{1-[2-cyclohexyl-2-oxoethyl]piperidin-4-yl}-6-fluoro-N-methyl-2-oxo-2,3-dihydro-1H-benzimidazole-5-carboxamide;
1-[1-(2-cyclohexyl-2-oxoethyl)piperidin-4-yl]-6-fluoro-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-6-fluoro-5-(5-oxo-4,5-dihydro-1,2,4-oxadiazol-3-yl)-1,3-dihydro-2H-benzimidazol-2-one;
N-methyl-1-{1-[2-(5-methylenehexahydrocyclopenta[c]pyrrol-2(1H)-yl)-2-oxoethyl]piperidin-4-yl}-2-oxo-2,3-dihydro-1H-benzimidazole-5-carboxamide;
1-(1-{2-[(3aR,6aS)-hexahydrocyclopenta[c]pyrrol-2(1H)-yl]-2-oxoethyl}piperidin-4-yl)-5-(5-methyl-1,2,4-oxadiazol-3-yl)-1,3-dihydro-2H-benzimidazol-2-one;
1-(1-{2-[(3aR,6aS)-hexahydrocyclopenta[c]pyrrol-2(1H)-yl]-2-oxoethyl}piperidin-4-yl)-5-[5-(trichloromethyl)-1,2,4-oxadiazol-3-yl]-1,3-dihydro-2H-benzimidazol-2-one;
1-{1-[2-(4-methoxy-4-methylpiperidin-1-yl)-2-oxoethyl]piperidin-4-yl}-6-methyl-5-(methylsulfonyl)-1,3-dihydro-2H-benzimidazol-2-one;
1-{1-[2-(4-methoxy-4-methylpiperidin-1-yl)-2-oxoethyl]piperidin-4-yl}-N-methyl-2-oxo-2,3-dihydro-1H-benzimidazole-5-carboxamide;
5-acetyl-1-{1-[2-(4-methoxy-4-methylpiperidin-1-yl)-2-oxoethyl]piperidin-4-yl}-1,3-dihydro-2H-benzimidazol-2-one;
5-acetyl-6-fluoro-1-{1-[2-(4-methoxy-4-methylpiperidin-1-yl)-2-oxoethyl]piperidin-4-yl}-1,3-dihydro-2H-benzimidazol-2-one;
1-{(1R,5S)-8-[2-(4,4-dimethylpiperidin-1-yl)-2-oxoethyl]-8-azabicyclo[3.2.1]oct-3-yl}-6-fluoro-N-methyl-2-oxo-2,3-dihydro-1H-benzimidazole-5-carboxamide;
5-acetyl-1-{1-[2-(6-azaspiro[2.5]oct-6-yl)-2-oxoethyl]piperidin-4-yl}-1,3-dihydro-2H-benzimidazol-2-one;
5-acetyl-1-{1-[2-(6-azaspiro[2.5]oct-6-yl)-2-oxoethyl]piperidin-4-yl}-6-fluoro-1,3-dihydro-2H-benzimidazol-2-one;
1-{1-[2-(4,4-dimethylpiperidin-1-yl)-2-oxoethyl]piperidin-4-yl}-5-(1,2,4-oxadiazol-3-yl)-1,3-dihydro-2H-benzimidazol-2-one;
6-fluoro-1-{(1R,5S)-8-[2-(hexahydrocyclopenta[c]pyrrol-2(1H)-yl)-2-oxoethyl]-8-azabicyclo[3.2.1]oct-3-yl}-N-methyl-2-oxo-2,3-dihydro-1H-benzimidazole-5-carboxamide;
1-{1-[2-(4,4-dimethylpiperidin-1-yl)-2-oxoethyl]piperidin-4-yl}-5-(ethylsulfonyl)-1,3-dihydro-2H-benzimidazol-2-one;
5-isobutyryl-1-{1-[2-(4-methoxy-4-methylpiperidin-1-yl)-2-oxoethyl]piperidin-4-yl}-1,3-dihydro-2H-benzimidazol-2-one;
1-{1-[2-(4-chlorophenyl)-2-hydroxyethyl]piperidin-4-yl}-5-(5-methyl-1,2,4-oxadiazol-3-yl)-1,3-dihydro-2H-benzimidazol-2-one;
1-{1-[2-(6-azaspiro[2.5]oct-6-yl)-2-oxoethyl]piperidin-4-yl}-5-isobutyryl-1,3-dihydro-2H-benzimidazol-2-one;
methyl 1-(1-{2-[(3aR,6aS)-hexahydrocyclopenta[c]pyrrol-2(1H)-yl]-2-oxoethyl}piperidin-4-yl)-2-oxoindoline-5-carboxylate;
1-{1-[2-(4-ethylcyclohexyl)-2-oxoethyl]piperidin-4-yl}-5-(methylsulfonyl)-1,3-dihydro-2H-benzimidazol-2-one;
6-fluoro-1-{1-[2-(4-isopropylcyclohexyl)-2-oxoethyl]piperidin-4-yl}-N-methyl-2-oxo-2,3-dihydro-1H-benzimidazole-5-carboxamide;
1-{1-[2-(4-methoxy-4-methylpiperidin-1-yl)-2-oxoethyl]piperidin-4-yl}-N-methyl-2-oxoindoline-5-carboxamide;
5-isobutyryl-1-{1-[2-(octahydro-2H-isoindol-2-yl)-2-oxoethyl]piperidin-4-yl}-1,3-dihydro-2H-benzimidazol-2-one;
1-{-[2-(6-azaspiro[2.5]oct-6-yl)-2-oxoethyl]piperidin-4-yl}-5-(methylsulfonyl)-1,3-dihydro-2H-benzimidazol-2-one;
1-{1-[2-(6-azaspiro[2.5]oct-6-yl)-2-oxoethyl]piperidin-4-yl}-N-methyl-2-oxoindoline-5-carboxamide;
6-fluoro-N-methyl-1-{1-[2-(1-methylcyclohexyl)-2-oxoethyl]piperidin-4-yl}-2-oxo-2,3-dihydro-1H-benzimidazole-5-carboxamide;
6-fluoro-1-{1-[2-(4-methoxycyclohexyl)-2-oxoethyl]piperidin-4-yl}-N-methyl-2-oxo-2,3-dihydro-1H-benzimidazole-5-carboxamide;
1-{1-[2-(4-methylcyclohexyl)-2-oxoethyl]piperidin-4-yl}-5-(methylsulfonyl)-1,3-dihydro-2H-benzimidazol-2-one;
1-{1-[2-(4-isopropylcyclohexyl)-2-oxoethyl]piperidin-4-yl}-5-(methylsulfonyl)-1,3-dihydro-2H-benzimidazol-2-one;
6-fluoro-N-methyl-1-{1-[2-(4-methylcyclohexyl)-2-oxoethyl]piperidin-4-yl}-2-oxo-2,3-dihydro-1H-benzimidazole-5-carboxamide;
5-(methylsulfonyl)-1-(1-{2-oxo-2-[4-(trifluoromethyl)cyclohexyl]ethyl}piperidin-4-yl)-1,3-dihydro-2H-benzimidazol-2-one;
1-{1-[2-(4,4-difluorocyclohexyl)-2-oxoethyl]piperidin-4-yl}-5-(methylsulfonyl)-1,3-dihydro-2H-benzimidazol-2-one;
6-fluoro-N-methyl-2-oxo-1-(1-{2-oxo-2-[4-(trifluoromethyl)cyclohexyl]ethylpiperidin-4-yl}-2,3-dihydro-1H-benzimidazole-5-carboxamide;
1-{1-[2-(4,4-difluorocyclohexyl)-2-oxoethyl]piperidin-4-yl}-6-fluoro-N-methyl-2-oxo-2,3-dihydro-1H-benzimidazole-5-carboxamide;
1-[2-(4,4-difluorocyclohexyl)-2-oxoethyl]piperidin-4-yl-6-fluoro-N-methyl-2-oxo-2,3-dihydro-1H-benzimidazole-5-carboxamide;
1-[1-(2-cyclohex-3-en-1-yl-2-oxoethyl)piperidin-4-yl]-6-fluoro-N-methyl-2-oxo-2,3-dihydro-1H-benzimidazole-5-carboxamide;
1-[1-(2-cyclohex-3-en-1-yl-2-oxoethyl)piperidin-4-yl]-N-methyl-2-oxoindoline-5-carboxamide;
1-{1-[2-(6-azaspiro[2.5]oct-6-yl)-2-oxoethyl]piperidin-4-yl}-6-fluoro-5-(methylsulfonyl)-1,3-dihydro-2H-benzimidazol-2-one;
1-{1-[2-(6-azaspiro[2.5]oct-6-yl)-2-oxoethyl]piperidin-4-yl}-6-methyl-5-(methylsulfonyl)-1,3-dihydro-2H-benzimidazol-2-one;
1-{-[2-(4-ethylcyclohexyl)-2-oxoethyl]piperidin-4-yl}-6-fluoro-N-methyl-2-oxo-2,3-dihydro-1H-benzimidazole-5-carboxamide;
6-fluoro-1-{1-[2-(4-methoxy-4-methylcyclohexyl)-2-oxoethyl]piperidin-4-yl}-N-methyl-2-oxo-2,3-dihydro-1H-benzimidazole-5-carboxamide;
1-[1-(2-cyclohexyl-2-hydroxyethyl)piperidin-4-yl]-6-fluoro-N-methyl-2-oxo-2,3-dihydro-1H-benzimidazole-5-carboxamide;
6-fluoro-N-methyl-1-{1-[2-(octahydro-2H-isoindol-2-yl)-2-oxoethyl]piperidin-4-yl}-2-oxoindoline-5-carboxamide;
methyl 1-{1-[2-(octahydro-2H-isoindol-2-yl)-2-oxoethyl]piperidin-4-yl}-2-oxoindoline-5-carboxylate;
1-[1-(2-cyclohexyl-2-oxoethyl)piperidin-4-yl]-N-methyl-2-oxoindoline-5-carboxamide;
N-methyl-1-{1-[2-(octahydro-2H-isoindol-2-yl)-2-oxoethyl]piperidin-4-yl}-2-oxoindoline-5-carboxamide;
1-[1-(2-cyclohexyl-2-hydroxyethyl)piperidin-4-yl]-N-methyl-2-oxoindoline-5-carboxamide;
methyl 1-{1-[2-(6-azaspiro[2.5]oct-6-yl)-2-oxoethyl]piperidin-4-yl}-2-oxoindoline-5-carboxylate;
1-{1-[2-(4-methoxy-4-methylpiperidin-1-yl)-2-oxoethyl]piperidin-4-yl}-5-propionyl-1,3-dihydro-2H-benzimidazol-2-one;
6-fluoro-N-methyl-1-{1-[2-(trans-4-methylcyclohexyl)-2-oxoethyl]piperidin-4-yl}-2-oxo-2,3-dihydro-1H-benzimidazole-5-carboxamide;
1-{1-[2-(4-ethylcyclohexyl)-2-hydroxyethyl]piperidin-4-yl}-5-(methylsulfonyl)-1,3-dihydro-2H-benzimidazol-2-one;
1-{1-[2-(6-azaspiro[2.5]oct-6-yl)-2-oxoethyl]piperidin-4-yl}-6-fluoro-N-methyl-2-oxoindoline-5-carboxamide;
methyl 1-{1-[2-(4-methoxy-4-methylpiperidin-1-yl)-2-oxoethyl]piperidin-4-yl}-2-oxoindoline-5-carboxylate;
1-[1-(2-cyclopentyl-2-oxoethyl)piperidin-4-yl]-6-fluoro-N-methyl-2-oxo-2,3-dihydro-1H-benzimidazole-5-carboxamide;
5-acetyl-1-{1-[2-(octahydro-2H-isoindol-2-yl)-2-oxoethyl]piperidin-4-yl}-1,3-dihydro-2H-benzimidazol-2-one;
6-fluoro-1-{1-[2-(4-methoxy-4-methylpiperidin-1-yl)-2-oxoethyl]piperidin-4-yl}-N-methyl-2-oxoindoline-5-carboxamide;
1-(1-{2-[(3aR,6aS)-hexahydrocyclopenta[c]pyrrol-2(1H)-yl]-2-oxoethyl}piperidin-4-yl)-2-oxoindoline-5-carboxylic acid;
1-[1-(2-cycloheptyl-2-oxoethyl)piperidin-4-yl]-6-fluoro-N-methyl-2-oxo-2,3-dihydro-1H-benzimidazole-5-carboxamide;
1-[1-(2-cyclohexyl-2-oxoethyl)piperidin-4-yl]-5-(1,2,4-oxadiazol-3-yl)-1,3-dihydro-2H-benzimidazol-2-one;
5-(5-methyl-1,2,4-oxadiazol-3-yl)-1-{1-[2-(4-methylpiperidin-1-yl)-2-oxoethyl]piperidin-4-yl}-1,3-dihydro-2H-benzimidazol-2-one;
5-(methylsulfonyl)-1-{1-[2-(octahydro-2H-isoindol-2-yl)-2-oxoethyl]piperidin-4-yl}-1,3-dihydro-2H-benzimidazol-2-one;
1-{1-[2-(6-azaspiro[2.5]oct-6-yl)-2-oxoethyl]piperidin-4-yl}-5-propionyl-1,3-dihydro-2H-benzimidazol-2-one;
1-{1-[2-(4,4-difluorocyclohexyl)-2-hydroxyethyl]piperidin-4-yl}-6-fluoro-N-methyl-2-oxo-2,3-dihydro-1H-benzimidazole-5-carboxamide;
1-{1-[2-(3,3-dimethylcyclobutyl)-2-oxoethyl]piperidin-4-yl}-N-methyl-2-oxoindoline-5-carboxamide;
6-fluoro-5-(methylsulfonyl)-1-{1-[2-(octahydro-2H-isoindol-2-yl)-2-oxoethyl]piperidin-4-yl}-1,3-dihydro-2H-benzimidazol-2-one;
1-{1-[2-(4-ethylcyclohexyl)-2-hydroxyethyl]piperidin-4-yl}-6-fluoro-N-methyl-2-oxo-2,3-dihydro-1H-benzimidazole-5-carboxamide;
1-{1-[2-bicyclo[3.1.]hex-6-yl]-2-oxoethyl)piperidin-4-yl}-6-fluoro-N-methyl-2-oxo-2,3-dihydro-1H-benzimidazole-5-carboxamide;
1-[1-(2-cyclopentyl-2-hydroxyethyl)piperidin-4-yl]-6-fluoro-N-methyl-2-oxo-2,3-dihydro-1H-benzimidazole-5-carboxamide;
1-[1-(3-cyclohexyl-2-oxopropyl)piperidin-4-yl]-6-fluoro-N-methyl-2-oxo-2,3-dihydro-1H-benzimidazole-5-carboxamide;
6-fluoro-1-{1-[2-hydroxy-2-(1,2,3,4-tetrahydronaphthalen-2-yl)ethyl]piperidin-4-yl}-N-methyl-2-oxo-2,3-dihydro-1H-benzimidazole-5-carboxamide;
6-fluoro-N-methyl-2-oxo-1-{1-[2-oxo-2-(1,2,3,4-tetrahydronaphthalen-2-yl)ethyl]piperidin-4-yl}-2,3-dihydro-1H-benzimidazole-5-carboxamide;
1-[1-(2-cycloheptyl-2-hydroxyethyl)piperidin-4-yl]-6-fluoro-N-methyl-2-oxo-2,3-dihydro-1H-benzimidazole-5-carboxamide;
6-fluoro-1-{1-[2-(4-methoxy-4-methylcyclohexyl)-2-oxoethyl]piperidin-4-yl}-N-methyl-2-oxo-2,3-dihydro-1H-benzimidazole-5-carboxamide;
6-fluoro-N-methyl-1-{1-[2-(octahydro-2H-isoindol-2-yl)-2-oxoethyl]piperidin-4-yl}-2-oxo-2,3-dihydro-1H-benzimidazole-5-carboxamide;
ethyl 1-{1-[2-(6-azaspiro[2.5]oct-6-yl)-2-oxoethyl]piperidin-4-yl}-2-oxo-2,3-dihydro-1H-benzimidazole-5-carboxylate;
1-{1-[2-(6-azaspiro[2.5]oct-6-yl)-2-oxoethyl]piperidin-4-yl}-2-oxo-2,3-dihydro-1H-benzimidazole-5-carboxylic acid;
1-{1-[2-(6-azaspiro[2.5]oct-6-yl)-2-oxoethyl]piperidin-4-yl}-6-fluoro-2-oxo-2,3-dihydro-1H-benzimidazole-5-carboxylic acid;
ethyl 2-oxo-1-[1-(2-oxo-2-spiro[2.5]oct-6-ylethyl)piperidin-4-yl]indoline-5-carboxylate;
ethyl 1-{1-[2-(6-azaspiro[2.5]oct-6-yl)-2-oxoethyl]piperidin-4-yl}-6-fluoro-2-oxo-2,3-dihydro-1H-benzimidazole-5-carboxylate;
6-fluoro-1-{1-[2-hydroxy-2-(4-methylcyclohexyl)ethyl]piperidin-4-yl}-N-methyl-2-oxo-2,3-dihydro-1H-benzimidazole-5-carboxamide;
methyl 1-{1-[2-(4,4-difluorocyclohexyl)-2-oxoethyl]piperidin-4-yl}-6-fluoro-2-oxoindoline-5-carboxylate;
6-fluoro-1-{1-[2-(cis-4-methoxy-4-methylcyclohexyl)-2-oxoethyl]piperidin-4-yl}-N-methyl-2-oxo-2,3-dihydro-1H-benzimidazole-5-carboxamide;
methyl 6-fluoro-1-(1-{2-[(3aR,6aS)-hexahydrocyclopenta[c]pyrrol-2(1H)-yl]-2-oxoethyl}piperidin-4-yl)-2-oxo-2,3-dihydro-1H-benzimidazole-5-carboxylate;
methyl 6-fluoro-1-(1-{2-[(3aR,6aS)-hexahydrocyclopenta[c]pyrrol-2(1H)-yl]-2-oxoethyl}piperidin-4-yl)-2-oxoindoline-5-carboxylate;
ethyl 6-fluoro-1-{1-[2-(4-methoxy-4-methylpiperidin-1-yl)-2-oxoethyl]piperidin-4-yl}-2-oxo-2,3-dihydro-1H-benzimidazole-5-carboxylate; and
ethyl 1-{1-[2-(4-methoxy-4-methylpiperidin-1-yl)-2-oxoethyl]piperidin-4-yl}-2-oxo-2,3-dihydro-1H-benzimidazole-5-carboxylate;
or a pharmaceutically acceptable salt, hydrate or solvate thereof.

6. A compound as recited in claim 2 wherein
E₂ is CRₐR_{b};
Rₐ and R_{b} are H;
R₂ is H or fluoro;
R₁ is —C(O)NR⁵R⁶;
R⁵ and R⁵ are H;
X is oxo;
R₃ and R₄ are H; and
A is octahydro-cyclopentapyrrole;
or a pharmaceutically acceptable salt thereof.

7. The compound 1-(1-{2-[hexahydrocyclopenta[c]pyrrol-2(1H)-yl]-2-oxoethyl}piperidin-4-yl)-N-methyl-2-oxoindoline-5-carboxamide or a pharmaceutically acceptable salt thereof.

8. The compound 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide or a pharmaceutically acceptable salt thereof.

9. The compound 1-(1-{2-[(3aR,6aS)-hexahydrocyclopenta[c]pyrrol-2(1H)-yl]-2-oxoethyl}piperidin-4-yl)-N-methyl-2-oxoindoline-5-carboxamide.

10. The compound having the structure.

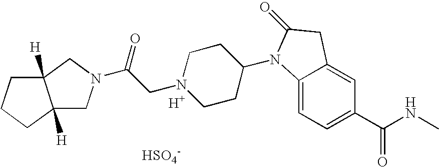

11. A pharmaceutical composition comprising a pharmaceutically effective amount of a compound of claim 1, or a pharmaceutically acceptable salt, hydrate or solvate form thereof, and a pharmaceutically acceptable carrier or excipient.

12. A method of treatment or amelioration of a disease selected from the group of type 2 diabetes, type 1 diabetes, insulin resistance, the effects of obesity, metabolic syndrome, Cushing's disease, impaired glucose tolerance, cardiovascular disease, atherosclerosis, prothrombotic conditions, myocardial infarction, hypertension, congestive heart failure, cardiomyopathy, atherosclerosis, dyslipidemia, sepsis, retinal degenerative disorders, emphysema, liver damage, cachexia, hepatitis C infections, HIV infections and inflammatory disorders, the method comprising administering to a mammal in need thereof a pharmaceutically effective amount of a compound of claim 1, or a pharmaceutically acceptable salt, hydrate or solvate form thereof.
